SEQUENCE LISTING (4 sequences)

SEQ ID NO: 1 - DNA, 20198 bp, Artificial Sequence
  SMN1 reporter construct
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgcttttccaaaccaccctccttttttgcgccctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacctaaccaagttcctctttcagaggttatttcaggccatggtgctgcgcagatccgcgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagcccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattaaccctcactaaagggaagcggccgcttacattttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccagctgctctatgccagcatttcctgcaaatgagaaattagaaccagaggcttgacgaattccagttaaaccatgtcctctgtggacaccagttaaacttgactagagcacttcatatgtcagagtgtacagtgcagtatgcctaggttatcccatatcacaataaaaaaaagtctgctggtctgcctactagtgatataaaatggcatcatatcctaaagctctttattgtgaaagtatgtttcttccacataaccaaccagttaagtatgagaattctagtagggatgtagattaaccttttatctaatagttttggcatcaaaattctttaatattgattgttttacattaacctttcaactttttaacatctgaactttttaaatgttcaaaaacatttgttttccacaaaccataaagttttacaaaagtaagattcactttcataatgctggcagacttactccttaattctaaggaatgtgagcaccttccttctttttgattttgtctgaaaccctgtaaggaaaataaaggaagttaaaaaaaatagctatatagacatagatagctatatatagatagctttatatggatgttaaaaagcattttgtttcacaagacattttacttattttattcaacaaaatatgatcagaaattaagttgatagtcttttaatgtactttaaaagttatcccaaagaaaacaattattaggctgcagttaaggttttcttgcagtggctcatgcctacaatcccacaactttgggaggcggaggtagggggatcacttgagacctggagcttgacaccaccctgggcaacataatgagaccctgtctctacaaaaaatttaaaaattaggccggcgtggtggctcaggctaggcacagtggctcacgcctgtaatcccagcactttgggaggccgagacagttggatcacctgagctcaggagttcgagaacagcctggccaacatggcaaaaccccatttctactgaaagtacaaaaaattagccaggcatggtggtggggacctctaatcccagctacttgggaggctgaggcaggagaatcacttgaacccaggaggcggaggctgcagtgagctgagatttacaccactgcactccagcctgggtgacagagcaagactctgtctcaaaaaaaaataaataaataaaaataaaaattagccaggtgcagtggcattatcactgtagtcccagctactcgggaaactgaggtgagaggactgcttgagccctggaggtcaaggctgcagtgagctgtgaatgtgcccttgcactccagcctgagcaatagagtgagacctggtctctaaaaaataaaatttgggaggcggagcttgcagtgagccgagactgcgccactgtactccagcctgggtgacagagcgagactccgtctcaaaaaaaaaaaaaaaataaaataaaataaaataattttaaatgttctgactaaaatacaatagaacatgtccgtaggagactaacgtataaagtgacaagtttgaagccatactccccaaggttcaatgtggtacacattaccccagatctttgtgcattaaaaaaatttcatttctcttggaaggccgaggcgggtggatcacggggtcaggagattgagaccatcctggctaacacagtgaaaccctgtctttacaaaaaaatacaaaaaattagacaggcgtggtggcaggcacctgtagtcccagctacctctgaggctgaggcaggagaatggcgtgaatccagaaggcagagcttgctgtgagccaagatcacgccattgcactccagcctgggcaacagagcaagactccgtctcaaaaaaaaaaaaaaaaaaaaaagaatttcatttttcatttatgaaaaattatcccatcttttccattccctacaatcaatttcaaatcagagattaaaacattatttagaaaaagtataatttcaattcaaaagtgtataatcaaaataatctaacaatagcatgaaagctttttaaaattaactaaaattatacttagggacaatgcaagagtaatttaagcctcagacagttgtatttttttatttttattttttagtaatataaagagagaagcaagtagtattttataaatttacaaaacaaagtcacataactacaaaaaaattgtcaggaaaagatgctgagtgattacttaccatataatagccagtatgatagccactcatgtaccatgaaattaacatacttcccaaagcatcagcatcatcaagagaatctggacatatgggaggtggtgggggaattatctcgagtggtccagaaggaaatggaggcagccagcatgatagtaagtggggtggtggtggtggcggtggcggtggtgggccattgaattttagacctggctttcctggtcccagtcttggccctggcatggggggtggtggagggagaaaagagttccatggagcagatttgggcttgatgttatctgatttatttccaggagacctggagttctcactttcatctgttgaaacttggctttcattttcattctcttgagcattctgttctatattattagctacttcacagattggggaaagtagatcggacagattttgctcctctctatttccatatccagtgtaaaccacaacacaggtttctctcttaaaatcaattgaagcaatggtagctgggtaaatgcaaccgtcttctgaccaaatggcagaacatttgtccccaactttccactgttgtaaggaagctgcagtattcttcttttggcttttattcttcttagcaggttttcttttaggtgtggtttttggtttacccgaagtttcacaaatgtcaccattctttagagcatgcttaaatgaagccacagctttatcatatgcttttatcagtgctgtatcatcccaaatgtcagaatcatcgctctggcctgtgccgcgccggaacagcacggaatcctcctgctccgggacgccgccaccactgccgccgctgctcatcgccatagcaaacccgcgggtgcgcagcgtggggccccgtcccttcttaagagtgacgacttccgccgcccggggcttctgggagcggaacagtacggtggccgggaggaccgcttgtagtaacttctcacgctttctacgagtggttatcgccctcccacatttgtggcgtgtatatttttcatttctctcaatcctttcatttcactgtgttatatttcctttccttttttttttgtttgtttgttttgagacagagcctcgccctgtcgctcaggctggagtgcagcggcgcgatctcggctcactgcagcctcgacttcttgggctcaagcgatcctcccacctcagcctccccagtagctaggactataggcgtgcgccaccacgctcagctattttttgtatttagtagagacggggtttcggcatgttgcttaggcctcgtctcgaactccagtgtgtgtgtgtgtgtgtgtgtgtgtgtgtgtgtgtgtgtagatatttattccccctcccccttggaaaagtaaatgtaagctcctactaggaatttaaaacctgcttgatctatataaagacaaacaaggaaagacaaacatgggggcaggaaggaaggcggcagatccttaaacactagaagatatttgatcccccaaccttatttgttgtttgttttgagacggagtctcgctctgtcgtcagagtgcagtggcaccatctcggctcattgcagcctcgacctcccgagctcaagcgatcctcccgcctcaacctcccaagtagctaggaccacaggggcacgccaccacacccggctagtttctgtatgttttgtagaggcggcgtttggagcatattgtgtaggctggtctcgaactcctgagctcaagatattccgcccgcctctggcatcccaaaatgctgggattacaggtgtgagccacctcgcccagcctccagtattcttttttttttttgcgacagagtattgctctgtcacccaggctggaatgcagtggcgtgatctcagctcactgcaacctctgcctcccaggttcaagcaattctcctgcctcagccccccgagtagctgggattacaggcgcccaccaccacacccggctaatttttgtatttttagtaaagatggggtttcaccatgttggccaggctggtcttgaactcctgaccttgtaatccgaccgcctcggcctcccaaagtgctgggattacaggtgtgagccaccacaccgggcctccagtattctttattaagcatctagggttgctaaatggcttatatgtacatagtatatatatatttttaactccacgaaaggaactttgagctcttcccccaaaatacccttggcttctatatagtatacaagaaatatctgtggaggaaggggagaatgggatgatgttgaccaagtgtacaaaaatggtaactccgtagaggtaatatgtggaatgtaatcatttcacaatgtatatctaaacatcaaatggtacaccttaaatatatacaatttttaggggtctggtacggtggctcatgcctataatcccagcactttgggaggccaaggtgggtggatcacttgaggtcaggagttcaagaccagcctggccaacatggtgaaaccctgtttctcctaaaaatacaaaaatcagccgggtgtggtggtgcaggcctgtaatgacagctgcttgggaggctgaggcaggagaatctcttgaactcgggaggcggaggttgcagtgagccaagatcacgccactgcactccagcctgagtgacagagtgcgactccatctcaaacaaataaatatgtacaatttttatgtgtcaaaaaagttaaattgtcacaagataaaaaaaaaaattttaaatctcatgtcaggaaagtaatgtgccaaaggtacatctcacagataaacatgaaaacctgcactccagcctgggcgacagagtgaggctgtgtctcagaaaaaaaaaaaaaagtaaaaaaaaaagtatgtttttataaagcttgcttagatttttctgaatcataaaaattctcacaattgcatttgatgtcaaaatttaaacaaattacgtggacatattacatgatggttaaaaaaataaatttaaacaaaatatagaaccaggtttctttttgttttttaatttttttctttttgagacggagtctcgctctgccacccagactggagtgcagtggctcactgcaacctctgcctcccgggttcaagtgattctcctgtctcagcttcccgagtacctgggattacaggcgtgtgccaccacgcccagctaatttttgtatttttagtagagacggggttttgccatgttggtcaggttggtctcaaactcctgaccttgtgatccgcccgcctcagcctcccaaagtgctgcgattacaggcatgagccaccgcacccagccatttctttttgtttttattatttagagatataattgatatactatagaattaatcgttttagagagtacaattgaatggtagatagagcggaaaccttaatatattcacaaggttgtgcaaccatcactactatctaactccagaacattttaatcacccaccaaagaaactctgtttcctttagcagtgcgctgccatgctcagctattttttgggagagaaggggtctccccatgttgtccacgctggtctcaaactcggttgcttaagcagtcctcccacttgagccgctgtgcccaggcctgagttactatatttataaaagttatttcatatgatagacaaatcattcaaaacataatgaggtaaactgccaaaagaaaccattttaccatatttgaaggcatttaatgtaaatgttgaatttaatttcatgtactggaatcagtctttttgcatatgtaattttcataccaaaaatctgtcttcagttgactcctggaactctctcatgataaaataaaagtttcaaataatgtcggggtggtggctaacacctgtaatcccagcactgtgggagtccgaggcaggtggatcacatgaggtcaggagtttgagaccagcctagccaacatggcaacactaaagatatgaaagtcagccaggcatggtggtgcatgcctgtaatctcagctactagggaggctgaggcacaaaaatcacttgaaactgggaggtggaggttgcaatgagctgagatcgtgccactgcacaccagcctgtgagacagagcaagactctgtctcaaaaaaaaaaaaaaaaaaaaaaaaaaaagggccaagtatggtggctcatgcctgtaatcctagcactttgggaggctgagtgggagaggatcatttgagcccaagtaacatggtcaggccccatctctacaaaaataaattagctgggcatggtggtatgggcttgtggtacatttatattggctcatgtccaatatgaccgccat

SEQ ID NO: 2 - DNA, 20200 bp, Artificial Sequence
  pCEP4-Rluc nSMN2 reporter construct
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccagctgctagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagcggccgctcgaggccggcaaggccggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcaggcgtcagcgggtgttggcgggtgtcggggcgcagccatgaggtcgactctagaggatcgatgccccgccccggacgaactaaacctgactacgacatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgcttttccaaaccaccctccttttttgcgccctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttgaagctgtccctgatggtcgtcatctacctgcctggacagcatggcctgcaacgcgggcatcccgatgccgccggaagcgagaagaatcataatggggaaggccatccagcctcgcgtcgcgaacgccagcaagacgtagcccagcgcgtcggccccgagatgcgccgcgtgcggctgctggagatggcggacgcgatggatatgttctgccaagggttggtttgcgcattcacagttctccgcaagaattgattggctccaattcttggagtggtgaatccgttagcgaggtgccgccctgcttcatccccgtggcccgttgctcgcgtttgctggcggtgtccccggaagaaatatatttgcatgtctttagttctatgatgacacaaaccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccggatcgggagatgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagccactggccccgtgggttagggacggggtcccccatggggaatggtttatggttcgtgggggttattattttgggcgttgcgtggggtcaggtccacgactggactgagcagacagacccatggtttttggatggcctgggcatggaccgcatgtactggcgcgacacgaacaccgggcgtctgtggctgccaaacacccccgacccccaaaaaccaccgcgcggatttctggcgtgccaagctagtcgaccaattctcatgtttgacagcttatcatcgcagatccgggcaacgttgttgccattgctgcaggcgcagaactggtaggtatggaagatctatacattgaatcaatattggcaattagccatattagtcattggttatatagcataaatcaatattggctattggccattgcatacgttgtatctatatcataatatgtacctaaccaagttcctctttcagaggttatttcaggccatggtgctgcgcagatccgcgtatgcggtgtgaaataccgcacagatgcgtaaggagaaaataccgcatcaggcgaaattgtaaacgttaatattttgttaaaattcgcgttaaatatttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagcccggatcgatccttatcggattttaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgctcgaagcattaaccctcactaaagggaagcggccgcttacattttacttacaatttggactttccgcccttcttggcctttatgaggatctctctgatttttcttgcgtcgagttttccggtaagacctttcggtacttcgtccacaaacacaactcctccgcgcaactttttcgcggttgttacttgactggcgacgtaatccacgatctctttttccgtcatcgtctttccgtgctccaaaacaacaacggcggcgggaagttcaccggcgtcatcgtcgggaagacctgccacgcccgcgtcgaagatgttggggtgttgtaacaatatcgattccaattcagcgggggccacctgatatcctttgtatttaattaaagacttcaagcggtcaactatgaagaagtgttcgtcttcgtcccagtaagctatgtctccagaatgtagccatccatccttgtcaatcaaggcgttggtcgcttccggattgtttacataaccggacataatcataggtcctctgacacataattcgcctctctgattaacgcccagcgttttcccggtatccagatccacaaccttcgcttcaaaaaatggaacaactttaccgaccgcgcccggtttatcatccccctcgggtgtaatcagaatagctgatgtagtctcagtgagcccatatccttgtcgtatccctggaagatggaagcgttttgcaaccgcttccccgacttctttcgaaagaggtgcgcccccagaagcaatttcgtgtaaattagataaatcgtatttgtcaatcagagtgcttttggcgaagaatgaaaatagggttggtactagcaacgcactttgaattttgtaatcctgaagggatcgtaaaaacagctcttcttcaaatctatacattaagacgactcgaaatccacatatcaaatatccgagtgtagtaaacattccaaaaccgtgatggaatggaacaacacttaaaatcgcagtatccggaatgatttgattgccaaaaataggatctctggcatgcgagaatctgacgcaggcagttctatgcggaagggccacacccttaggtaacccagtagatccagaggaattcattatcagtgcaattgttttgtcacgatcaaaggactctggtacaaaatcgtattcattaaaaccgggaggtagatgagatgtgacgaacgtgtacatcgactgaaatccctggtaatccgttttagaatccatgataataattttctggattattggtaattttttttgcacgttcaaaattttttgcaacccctttttggaaacaaacactacggtaggctgcgaaatgttcatactgttgagcaattcacgttcattataaatgtcgttcgcgggcgcaactgcaactccgataaataacgcgcccaacaccggcataaagaattgaagagagttttcactgcatacgacgattctgtgatttgtattcagcccatatcgtttcatagcttctgccaaccgaacggacatttcgaagtattccgcgtacgtgatgttcacctcgatatgtgcatctgtaaaagcaattgttccaggaaccagggcgtatctcttcatagccttatgcagttgctctccagcggttccatcctctagaggatagaatggcgccgggcctttctttatgtttttggcgtcttccagctgctctatgccagcatttcctgcaaatgagaaattagaaccagaggcttgacgaattccagttaaaccatgtcctctgtggacaccagttaaacttgactagagcacttcatatgtcagagtgtacagtgcagtatgcctaggttatcccatatcacaataaaaaaaagtctgctggtctgcctactagtgatataaaatggcatcatatcctaaagctctttattgtgaaagtatgtttcttccacacaaccaaccagttaagtatgagaattctagtagggatgtagattaaccttttatctaatagttttggcatcaaaattctttaatattgattgttttacattaacctttcaactttctaacatctgaactttttaaatgttcaaaaacatttgttttccacaaaccataaagttttacaaaagtaagattcactttcataatgctggcagacttactccttaatcttaaggaatgtgagcaccttccttctttttgattttgtctaaaaccctgtaaggaaaataaaggaagttaaaaaaaatagctatatagatatagatagctatatatagatagctttatatggatgttaaaaagcattttgtttcacaagacattttacttattttattcaacaaaatatgatcagaaattaagttgatagtcttttaatgtactttaaaagttatcccaaagaaaacaattattaggctgcagttaaggttttcttgcagtggctcatgcctacaatcccacaactttgggaggcggaggtagggggatcacttgagacctggagcttgacaccaccctgggcaacataatgagaccctgtctctacaaaaaatttaaaaattaggccggcgtggtggctcatgctaggcacagtggctcacgcctgtaatcccagcactttgggaggccgagacagttggatcacctgagctcaggagttcgagaacagcctggccaacatggcaaaaccccgtttctactgaaagtacaaaaaattagccaggcatggtggtggggacctctaatcccagctacttgggaggttgaggcaggagaatcacttgaacccaggaggcggaggctgcagtgagctgagatttacaccactgcactccagcctgggtgacagagcaagactctgtctcaaaaaaaaataaataaataaaaataaaaattagccaggtgcagtggcattatcactgtagtcccagctactcgggaaactgaggtgagaggactgcttgagccctggaggtcaaggctgcagtgagctgtgaatgtgcccttgcactccagcctgagcaatagagtgagacctggtctctaaaaaataaaatttgggaggcggagcttgcagtgagccgagactgcgccactgtactccagcctgggtgacagagcgagactccgtctcaaaaaaaaaaaaaaaataaaataaaataaaataattttaaatgttctgactaaaatacaatagaacatgtccgtaggagactaacgtataaagtgacaagtttgaagccatactccccaaggttcaatgtggtacacattaccccagatctttgtgcattaaaaaaatttcatttctcttggaaggccgaggcgggtggatcacggggtcaggagattgagaccatcctggctaacacagtgaaaccctgtctttacaaaaaaatacaaaaaattagacaggcgtggtggcaggcaactgtagtcccagctacctctgaggctgaggcaggagaatggcgtgaatccaggaggcagagcttgctgtgagccaagatcacgccattgcactccagcctgggcaacagagcaagactccgtctcaaaaaaaaaaaaaaaaaaaaagaatttcatttttcatttatgaaaaattatcccatcttttccattccctacaatcaatttcaaatcagagattaaaacattatttagaaaaagtataatttcaattcaaaagtgtataatcaaaataatctaacaatagcatgaaagctttttaaaattaactaaaattatacttagggacaatgcaagagtaatttaagcctcagacagttgtatttttttatttttattttttagtaatataaagagagaagcaagtagtattttataaatttacaaaacaaagtcacataactacaaaaaaattgtcaggaaaagatgctgagtgattacttaccatataatagccagtatgatagccactcatgtaccatgaaattaacatacttcccaaagcatcagcatcatcaagagaatctggacatatgggaggtggtgggggaattatctcgagtggtccagaaggaaatggaggcagccagcatgatagtaagtggggtggtggtggtggcggtggcggtggtgggccattgaattttagacctggctttcctggtcccagtcttggccctggcatggggggtggtggagggagaaaagagttccatggagcagatttgggcttgatgttatctgatttatttccaggagacctggagttctcactttcatctgttgaaacttggctttcattttcattctcttgagcattctgttctatattattagctacttcacagattggggaaagtagatcggacagattttgctcctctctatttccatatccagtgtaaaccacaacacaggtttctctcttaaaatcaattgaagcaatggtagctgggtaaatgcaaccgtcttctgaccaaatggcagaacatttgtccccaactttccactgttgtaaggaagctgcagtattcttcttttggcttttattcttcttagcaggttttcttttaggtgtggtttttggtttacccgaagtttcacaaatgtcaccattctttagagcatgcttaaatgaagccacagctttatcatatgcttttatcagtgctgtatcatcccaaatgtcagaatcatcgctctggcctgtgccgcgccggaacagcacggaatcctcctgctccgggacgccgccaccactgccgccgctgctcatcgccatagcaaacccgcgggtgcgcagcgtggggccccgtcccttcttaagagtgacgacttccgccgcccggggcttctgggagcggaacagtacggtggccgggaggaccgcttgtagtaacttctcacgctttctacgagtggttatcgccctcccacatttgtggcgtgtatatttttcatttctctcaatcctttcatttcactgtgttatatttcctttccttttttttttgtttgtttgttttgagacagagcctcgccctgtcgctcaggctggagtgcagcggcgcgatctcggctcactgcagcctcgacttcttgggctcaagcgatcctcccacctcagcctccccagtagctaggactataggcgtgcgccaccaagctcagctattttttgtatttagtagagacggggtttcggcatgttgcttaggcctcgtctcgaactccagtgtgtgtgtgtgtgtgtgtgtgtgtgtgtgtgtgtgtgtgtgtgtagatatttattccccctcccccttggaaaagtaaatgtaagctcctactaggaatttaaaacctgcttgatctatataaagacaaacaaggaaagacaaacatgggggcaggaaggaaggcagatccttaaacactagaagatatttgatcccccaaccttatttgttgtttgttttgagacggagtctcgctctgtcgtcagagtgcagtggcaccatctcggctcattgcagcctcgacctcccgagctcaagcgatcctcccgcctcaacctcccaagtagctaggaccacaggggcacgccaccacacccggctagtttctgtatgttttgtagaggcggcgtttggagcatattgtgtaggctggtctcgaactcctgagctcaagatattccgcccgcctctggcatcccaaaatgctgggattacaggtgtgagccacctcgcccagcctccagtattcttttttttttttgcgacagagtattgctctgtcacccaggctggaatgcagtggcgtgatctcagctcactgcaacctctgcctcccaggttcaagcaattctgcctcagccccccgagtagctgggattacaggcgcccaccaccacacccggctaatttttgtatttttagtaaagatggggtttcaccatgttggccaggctggtcttgaactcctgacctcgtaatccgaccgcctcggcctcccaaagtgctgggattacaggtgtgagccaccacaccgggcctccagtattctttattaagcatctagggttgctaaatggcttatatgtacatagtatatatatatttttaactccacgaaaggaactttgagctcttcccccaaaatacccttggcttctatatagtatacaagaaatatctgtggaggaaggggagaatgggatgatgttgaccaagtgtacaaaaatggtaactctgtagaggtaatatgtggaatgtaatcatttcacaatgtatatctaaacatcaaatggtacaccttaaatatatacaatttttaggggtctggtacggtggctcatgcctataatcccagcactttgggaggccaaggtgggtggatcacttgaggtcaggacttcaagaccagcctggccaacatggtgaaaccctgtttctcctaaaaatacaaaaatcagccgggtgtggtggtgcaggcctgtaatgacagctgcttgggaggctgagccaggagaatcacttgaactcgggaggcggaggttgcagtgagccaagatcacgccactgcactccagcctgagtgacagagtgcgactccatctcaaacaaataaatatgtacaatttttatgtgtcaaaaaagttaaattgtcacaagataaaaaaaaaaatttaaatctcatgtcaggaaagtaatgtgccaaaggtacatctcacagataaacatgaaaacctgcactccagcctgggcgacagagtgaggctgtgtctcagaaaaaaaaaaaaaagtaaaaaaaaaagtatgtttttataaagcttgcttagatttttctgaatcataaaaattctcacaattgcatttgatgtcaaaatttaaacaaattacctggacatattacatgatggttaaaaaaataaatttaaacaaaatatagaaccaggtttctttttgttttttaatttttttctttttgagacggagtctcgctctgccacccagactggagtgcagtggctcactgcaacctctgcctcccgggttcaagtgattctcctgtctcagcttcccgagtacctaggattacaggcgtgtgccaccacacccagctaatttttgtatttttagtagagactgggttttgccatgttggtcaggttggtctcaaactcctgaccttgtgatccgcccgcctcagcctcccaaagtgctgcgattacaggcatgagccaccgcacccagccatttctttttgtttttattatttagagatataattgatatactatagaattaatcgttttagagagtacaattgaatggtagatagagcggaaaccttaatatattcacaaggttgtgcaaccatcactactatctaactccagaacattttaatcacccaccaaagaaactctgtttcctttagcagtgcgctgccatgctcagctattttttgggagagaaggggtctccccatgttgtccacgctggtctcaaactcggttgcttaagcagtcctcccacttgagccgctgtgcccaggcctgagttactatatttataaaagttatttcatatgatagacaaatcattcaaaacataatgaggtaaactgccaaaagaaaccattttaccatatttgaaggcatttaatgtaaatgttgaatttaatttcatgtactggaatcagtctttttgcatatgtaattttcataccaaaaatctctcttcagttgactcctggaactctctcatgataaaataaaagtttcaaataatgtcggggtggtggctaacacctgtaatcccagcactgtgggagtccgaggcaggtggatcacatgaggtcaggagtttgagaccagcctagccaacatggcaacactaaagatatgaaagtcagccaggcatggtggtgcatgcctgtaatctcagctactagggaggctgaggcacaaaaatcacttgaaactgggaggtggaggttgcaatgagctgagatcgtgccactgcacaccagcctgtgagacagagcaagactctgtctcaaaaaaaaaaaaaaaaaaaaaaaaaaaagggccaagtatggtggctcatgcctgtaatcctagcactttgggaggctgagtgggagaggatcatttgagcccaagtaacatggtcaggccccatctctacaaaaataaattagctgggcatggtggtatgggcttgtggtacatttatattggctcatgtccaatatgaccgccat

SEQ ID NO: 3 - DNA, 34 bp, Artificial Sequence
  forward primer
cacccgcgggtttgctatggcgatgagcagcggc

SEQ ID NO: 4 - DNA, 35 bp, Artificial Sequence
  reverse primer
tatctcgagtggtccagaaggaaatggaggcagcc

(19) United States
(12) Patent Application Publication
(10) Pub. No.: US 2013/0096160 A1
(43) Pub. Date: Apr. 18, 2013

(54) ARYLTHIAZOLYL PIPERIDINES AND RELATED COMPOUNDS AS MODULATORS OF SURVIVAL MOTOR NEURON (SMN) PROTEIN PRODUCTION

(71) Applicants: Juan Jose Marugan, Gaithersburg, MD (US); Jingbo Xiao, Rockville, MD (US); Steven A. Titus, Elkridge, MD (US); Noel Southall, Chevy Chase, MD (US); Wei Zheng, Potomac, MD (US); Elliot J. Androphy, Natick, MA (US); Jonathan Cherry, Worcester, MA (US)

(72) Inventors: Juan Jose Marugan, Gaithersburg, MD (US); Jingbo Xiao, Rockville, MD (US); Steven A. Titus, Elkridge, MD (US); Noel Southall, Chevy Chase, MD (US); Wei Zheng, Potomac, MD (US); Elliot J. Androphy, Natick, MA (US); Jonathan Cherry, Worcester, MA (US)

(73) Assignees: Secretary, Department of Health and Human Services, Bethesda, MD (US); THE UNITED STATES OF AMERICA, as represented by the

(21) Appl. No.: 13/641,383

(22) PCT Filed: Apr. 14, 2011
(86) PCT No.: PCT/US11/32501

§ 371 Date: Jan. 7, 2013

Related U.S. Application Data
(60) Provisional application No. 61/323,963, filed on Apr. 14, 2010.

(51) Int. Cl. C07D 417/04

(52) U.S. Cl. CPC C07D 417/04
USPC 514/326; 546/209

(57) ABSTRACT

Aryl substituted thiazol-2-yl-piperidines and related compounds useful as modulators of survival motor neuron (SMN) protein production are provided herein. Without being bound to any particular theory it is believed the aryl substituted thiazol-2-yl-piperidines and related compounds provided herein act to increase production of the SMN2 form of survival motor neuron protein. These compounds are useful for treating spinal muscular atrophy. Pharmaceutical compositions containing a carrier and one or more of the aryl substituted thiazol-2-yl-piperidine or related compounds described herein are also provided. Methods of treating spinal muscular atrophy are also provided by this disclosure.

### CROSS REFERENCE TO RELATED APPLICATION

This application claims priority from U.S. Provisional Patent application No. 61/323,963, filed Apr. 14, 2010, which is hereby incorporated by reference in its entirety.

### STATEMENT OF GOVERNMENT SUPPORT

This invention was made in part with government support from the National Institutes of Health. The government has certain rights in this invention.

### FIELD OF THE DISCLOSURE

Aryl substituted thiazol-2-yl-piperidines and related compounds useful as modulators of survival motor neuron (SMN) protein production are provided herein. Without being bound to any particular theory it is believed the aryl substituted thiazol-2-yl-piperidines and related compounds provided herein act to increase production of the SMN2 form of survival motor neuron protein. These compounds are useful for treating spinal muscular atrophy. Pharmaceutical compositions containing a carrier and one or more of the aryl substituted thiazol-2-yl-piperidine or related compounds described herein are also provided. Methods of treating spinal muscular atrophy are also provided by this disclosure.

### BACKGROUND

Spinal muscular atrophy (SMA) refers to a group of neuromuscular disorders characterized by degeneration of lower motor neurons of the anterior horn cells of the spinal cord due to reduced survival motor neuron (SMN) protein, leading to symmetrical muscle weakness and atrophy. SMA types are classified according to the age of onset, maximum muscular activity achieved, and survivorship. All SMA types are caused by recessive mutations in the SMN1 gene.

SMA is the second most common lethal, autosomal recessive disease in Caucasians after cystic fibrosis, and is the leading genetic cause of infant mortality in the United States and Western Europe, with an incidence of 1 in 6000 live births and a carrier frequency of about 1 in 40. Currently, although several drugs are under clinical investigation for treatment of SMA, there is no approved drug treatment for this orphan genetic disease. Presently, treatment for SMA consists of prevention and management of the secondary effect of chronic motor unit loss.

SMN protein is expressed as a 294 a polypeptide that is processed further to obtain the mature protein. It is expressed in a wide variety of tissues throughout the body, with high levels found in spinal cord. SMN protein is involved in maintenance of specialized nerve cells called motor neurons, which are located in the spinal cord and the part of the brain that is connected to the spinal cord (the brainstem) and control muscle movement.

Additionally, SMN protein plays an important role in processing messenger RNA (mRNA). It is part of a complex that plays an essential role in spliceosomal snRNP assembly in the cytoplasm and is required for pre-mRNA splicing in the nucleus.

In humans, there are two nearly identical copies of the gene encoding for SMN protein, SMN1 and SMN2 SMN1 and SMN2 lie within the telomeric and centromeric halves, respectively, of a large inverted repeat on chromosome 5q. Most SMN protein is expressed from the SMN1 gene. The coding sequence of SMN2 differs from that of SMN1 by a single nucleotide in exon 7 (840C-T), which results in decreased transcription of full-length SMN mRNA from the SMN2 gene and predominantly produces transcripts without exon 7 and other splice variants. The smaller, nonfunctional polypeptides translated from these splice variants are readily degraded.

About 95 percent of individuals with spinal muscular atrophy have mutations that delete exon 7 in both copies of this gene. As a result, little or no SMN protein is made. In about 5 percent of people with this disorder, one copy of the SMN1 gene has a deletion of exon 7, and the other copy has a different mutation that disrupts the production or function of the SMN protein. Researchers have identified at least 65 mutations in the SMN1 gene that cause spinal muscular atrophy.

Changes in expression of the centromeric copy of SMN, SMN2, as well as the copy number of SMN2 are known to modify the disease phenotype. In humans, there is a direct correlation between SMN2 copy number and disease severity, with low copy numbers found in early onset SMA and higher copy numbers in less severe and delayed forms. (Vitali T et al. Detection of the survival motor neuron (SMN) genes by FISH: further evidence for a role for SMN2 in the modulation of disease severity in SMA patients. Hum Mol Genet. 1999; 8: 2525-2532). These results in humans are consistent with results in SMA mouse models. A single copy of human SMN2 was sufficient to restore viability to a SMN−/− animal, although severe SMA develops and mice die within days after birth. Further, mice with eight copies of human SMN2 are phenotypically normal.

Since level of SMN protein expression correlates with SMA disease severity, compounds that upregulate expression of full-length SMN from SMN2 are desirable therapeutic candidates for SMA.

### SUMMARY

In a first aspect compounds and pharmaceutically acceptable salts of the following Formula I and Formula II are provided herein.

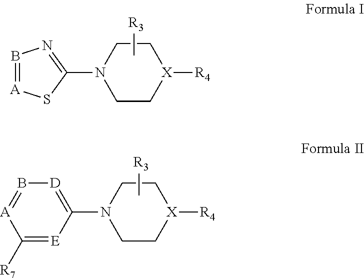

A, B, D, E, X, R₃, R₄, and R₇ in Formula I are defined as follows.

X is Nor CH.

One of A and B is C—R₁ and the other of A and B is N or C—R₂.

D is N or C—R₅, where R₅ is hydrogen, halogen, C₁-C₂alkyl, or C₁-C₂alkoxy.

E is N or C—R₆, where R₆ is hydrogen, halogen, C₁-C₂alkyl, or C₁-C₂alkoxy. R₇ is hydrogen, halogen, C₁-C₂alkyl, or C₁-C₂alkoxy.

R₁ is an optionally substituted mono-, bi-, or tricyclic group having at least one aromatic or hetero aromatic ring.

R₂ is hydrogen, halogen, C₁-C₂alkyl, C₁-C₂alkoxy, trifluoromethyl, or trifluoromethoxy.

R₃ is 1 to 4 substituents independently chosen from hydrogen, halogen, C₁-C₂alkyl, and C₁C₂alkoxy.

R₄ is cyano, amino, (C═O)R₁₀, —(C═O)NR₁₀R₁₁, (C═O)OR₁₀, —NR₁₂(C═O)R₁₀, —NR₁₂(C═O)OR₁₀, or a 5-membered heteroaryl group containing at least 2 nitrogen atoms; wherein R₁₀ and R₁₁ are independently chosen from hydrogen, C₁-C₄alkyl, (cycloalkyl)C₀-C₄alkyl, (heterocycloalkyl)C₀-C₄alkyl, (phenyl)C₀-C₄alkyl, and (pyridyl)C₀-C₄alkyl; and R₁₂ is hydrogen or C₁-C₂alkyl.

Provided herein are compounds and salts of Formula I and II that are potent and selective modulators of SMN protein production.

Pharmaceutical compositions comprising a carrier and one or more compounds or salts of Formula I or Formula II are also provided herein.

Methods of spinal muscular atrophy in a patient comprising administrating a compound or salt of Formula I or Formula II to the patient are provided herein.

### BRIEF DESCRIPTION OF THE DRAWINGS

FIG. 1 presents the percent luciferase activity observed in the reporter gene assay as a function of compound concentration for compounds MLS000763654 (left panel) and MLS0006988454 (right panel) for the SMN2-luciferase reporter (SMN2-luc, upper line), the SMN1-luciferase reporter (SMN1-luc, lower line in left panel, center line in right panel), and the luciferase reporter.

FIG. 2 shows images of the Western blot analysis of SMN protein present in a sample of SMA carrier cells (3814) or of SMA patient cells (3813) cultured in the absence or presence of 0.1, 1, or 10 μM MLS00069884 or MLS000763654.

FIG. 3 is a map of the SMN2 reporter construct used in the qHTS assay.

FIG. 4. Quantification of western blot of SMN levels after treatment with drug compounds as indicated with different doses. 3814 is a fibrolast cell line control, 3813 is a fibrolast cell line from SMA patients. 8a: XJB03-055, 8c: XJB03-049, 81: XJB03-054, 8m: XJB03-068, 9a: XJB04-008, 9c: XJB04-011-C

FIG. 5. Number of gems per 100 nuclei after treatment with drug compounds as indicated with different doses.

### DETAILED DESCRIPTION

### Terminology

Prior to setting forth the invention in detail, it may be helpful to provide definitions of certain terms to be used herein. Compounds of the present invention are described using standard nomenclature. Unless defined otherwise, all technical and scientific terms used herein have the same meaning as is commonly understood by one of skill in the art to which this invention belongs.

Formula I and Formula II includes all subformulae thereof. For example Formula I includes compounds of Formulas III to V and Formula II includes compounds of Formulas VI to X and the pharmaceutically acceptable salts and hydrates thereof.

The terms “a” and “an” do not denote a limitation of quantity, but rather denote the presence of at least one of the referenced items. The term “or” means “and/or”. The terms “comprising”, “having”, “including”, and “containing” are to be construed as open-ended terms (i.e., meaning “including, but not limited to”). Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein. Unless defined otherwise, technical and scientific terms used herein have the same meaning as is commonly understood by one of skill in the art to which this invention belongs.

An “active agent” means a compound (including a compound of Formula I or II), element, or mixture that when administered to a patient, alone or in combination with another compound, element, or mixture, confers, directly or indirectly, a physiological effect on the patient. The indirect physiological effect may occur via a metabolite or other indirect mechanism. Salts, solvates (including hydrates) of the compound of Formula I (or II), crystalline forms, non-crystalline forms, and any polymorphs of the compound are included. Compounds may contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds may occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

All forms (for example solvates, optical isomers, enantiomeric forms, polymorphs, free compound and salts) of an active agent may be employed either alone or in combination.

In certain situations, the compounds of Formula I (or II) may contain one or more asymmetric elements such as stereogenic centers, including chiral centers, stereogenic axes and the like, e.g. asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, it should be understood that all of the optical isomers and mixtures thereof are encompassed. In addition, compounds with carbon-carbon double bonds may occur in Z- and E-forms, with all isomeric forms of the compounds being included in the present invention. Formula I includes all chiral forms, stereoisomers, diastereomers, and enantiomers of compounds of Formula I.

Stereochemical definitions and conventions used herein generally follow S. P. Parker, Ed., McGraw-Hill Dictionary of Chemical Terms (1984) McGraw-Hill Book Company, New York; and Eliel, E. and Wilen, S., Stereochemistry of Organic Compounds (1994) John Wiley & Sons, Inc., New York. Many organic compounds exist in optically active forms, i.e., they have the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or 1 meaning that the compound is levorotatory. A compound prefixed with (+) or d is dextrorotatory.

A “racemic mixture” or “racemate” is an equimolar (or 50:50) mixture of two enantiomeric species, devoid of optical activity. A racemic mixture may occur where there has been no stereoselection or stereospecificity in a chemical reaction or process.

Where a compound exists in various tautomeric forms, the invention is not limited to any one of the specific tautomers, but rather includes all tautomeric forms.

The invention includes compounds of Formula I (and II) having all possible isotopes of atoms occurring in the compounds. Isotopes include those atoms having the same atomic number but different mass numbers. By way of general example, and without limitation, isotopes of hydrogen include tritium and deuterium and isotopes of carbon include ¹¹C, ¹³C, and ¹⁴C.

Certain compounds are described herein using a general formula that includes variables, e.g. A, B, D, E, R₃, R₄, and R₇. Unless otherwise specified, each variable within Formula I (and II) is defined independently of other variables. Thus, if a group is said to be substituted, e.g. with 0-2 R*, then said group may be substituted with up to two R* groups and R* at each occurrence is selected independently from the definition of R*. Also, combinations of substituents and/or variables are permissible only if such combinations result in stable compounds.

The term “substituted”, as used herein, means that any one or more hydrogens on the designated atom or group is replaced with a selection from the indicated group, provided that the designated atom's normal valence is not exceeded. When the substituent is oxo (i.e., ═O), then 2 hydrogens on the atom are replaced. When aromatic moieties are substituted by an oxo group, the aromatic ring is replaced by the corresponding partially unsaturated ring. For example a pyridyl group substituted by oxo is a pyridone. Combinations of substituents and/or variables are permissible only if such combinations result in stable compounds or useful synthetic intermediates. A stable compound or stable structure is meant to imply a compound that is sufficiently robust to survive isolation from a reaction mixture, and subsequent formulation into an effective therapeutic agent.

A dash (“-”) that is not between two letters or symbols is used to indicate a point of attachment for a substituent. For example, —NR₁₂(C═O)R₁₀ is bound via the nitrogen.

“Alkyl” includes both branched and straight chain saturated aliphatic hydrocarbon groups, having the specified number of carbon atoms, generally from 1 to about 12 carbon atoms. The term C₁-C₆alkyl as used herein indicates an alkyl group having from 1 to about 6 carbon atoms. When C₀-Cₙ alkyl is used herein in conjunction with another group, for example, (phenyl)C₀-C₂ alkyl, the indicated group, in this case phenyl, is either directly bound by a single covalent bond (C₀), or attached by an alkyl chain having the specified number of carbon atoms, in this case from 1 to about 2 carbon atoms. Examples of alkyl include, but are not limited to, methyl, ethyl, n-propyl, isopropyl, n-butyl, 3-methylbutyl, t-butyl, n-pentyl, and sec-pentyl.

“Alkanoyl” is an alkyl group as defined above, attached through a keto (—(C═O)—) bridge. Alkanoyl groups have the indicated number of carbon atoms, with the carbon of the keto group being included in the numbered carbon atoms. For example a C₂alkanoyl group is an acetyl group having the formula —(C═O)CH₃.

“Alkoxy” means an alkyl group, as defined above, with the indicated number of carbon atoms attached via an oxygen bridge. Examples of alkoxy include, but are not limited to, methoxy, ethoxy, isopropoxy, and 3-methylpentoxy.

“Mono- and/or di-alkylamino” indicates secondary or tertiary alkyl amino groups, wherein the alkyl groups are as defined above and have the indicated number of carbon atoms. The point of attachment of the alkylamino group is on the nitrogen. The alkyl groups are independently chosen. Examples of mono- and di-alkylamino groups include ethylamino, dimethylamino, and methyl-propyl-amino. “Mono- and/or dialkylaminoalkyl” groups are mono- and/or di-alkylamino groups attached through an alkyl linker having the specified number of carbon atoms, for example a di-methylaminoethyl group. Tertiary amino substituents may by designated by nomenclature of the form N—R—N—R′, indicating that the groups R and R′ are both attached to a single nitrogen atom.

The term “alkylthio” indicates an alkyl group as defined above attached through a sulfur linkage, i.e. a group of the formula alkyl-S—. Examples include methylthio, ethylthio, and pentylthio.

“Aryl” means aromatic groups containing only carbon in the aromatic ring or rings. Typical aryl groups contain 1 to 3 separate, fused, or pendant rings and from 6 to about 18 ring atoms, without heteroatoms as ring members. When indicated, such aryl groups may be further substituted with carbon or non-carbon atoms or groups. Such substitution may include fusion to a 5 to 7-membered saturated cyclic group that optionally contains 1 or 2 heteroatoms independently chosen from N, O, and S, to form, for example, a 3,4-methylenedioxy-phenyl group. Aryl groups include, for example, phenyl, naphthyl, including 1-naphthyl and 2-naphthyl, and bi-phenyl.

“Mono- and/or di-alkylcarboxamide” indicates groups of formula (alkyl₁)—NH—(C═O)— and (alkyl₁)(alkyl₂)—N—(C═O)— in which the alkyl₁ and alkyl2 groups are independently chosen alkyl groups as defined above having the indicated number of carbon atoms. Mono and/or di-alkylcarboxamide also refers to groups of the formula NH(C═O)(alkyl₁) and N(alkyl₂)(C═O)(alkyl₁), carboxamide groups in which the point of attachment is the nitrogen atom, in which the alkyl₁ and alkyl₂ groups are independently chosen alkyl groups as defined above having the indicated number of carbon atoms.

“Mono- and di-alkylsulfonamide” means groups of the formula (alkyl₁)—NH—(SO₂)— and (alkyl₁)(alkyl₂)—N-(50₂)— in which the alkyl₁ and alkyl₂ groups are independently chosen alkyl groups as defined above having the indicated number of carbon atoms. Mono and/or di-alkylcarboxamide also refers to groups of the formula NH(SO₂)(alkyl₁) and N(alkyl₂)(SO₂)(alkyl₁), sulfonamide groups in which the point of attachment is the nitrogen atom, in which the alkyl₁ and alkyl₂ groups are independently chosen alkyl groups as defined above having the indicated number of carbon atoms.

“Alkylsulfonyl” means alkyl-(SO₂)—, where the alkyl group is an alkyl group as defined above having the defined number of carbon atoms. An exemplary alkylsulfonyl group is methylsulfonyl.

“Cycloalkyl” indicates saturated hydrocarbon ring groups, having the specified number of carbon atoms, usually from 3 to about 8 ring carbon atoms, or from 3 to about 6 carbon atoms. Examples of cycloalkyl groups include cyclopropyl, cyclobutyl, cyclopentyl, or cyclohexyl as well as bridged or caged saturated ring groups such as norborane or adamantane.

A “mono-, bi-, or tricyclic group having at least one aromatic or heteroaromatic ring” is a group having aryl or heteroaryl ring or 2 or 3 fused rings in which at least one ring is aromatic (has 4n+2 delocalized electrons) and the remaining ring or rings are aromatic, saturated or unsaturated. Rings may have from 4 to 7 ring atoms, or in certain embodiments from 5 to 7 ring atoms. Rings may contain 1, 2, 3, or 4 heteroatoms independently chosen from N, O, and S with the remaining ring atoms being carbon. When the total number of S and O atoms in the heteroaryl group exceeds 1, these heteroatoms are not adjacent to one another. It is preferred that the total number of S and O atoms in the heteroaryl group is not more than 2. It is particularly preferred that the total number of S and O atoms in the heteroaryl group is not more than 1. When indicated, such cyclic groups may be further substituted with carbon or non-carbon atoms or groups. Examples of mono, bi- or tricyclic groups having at least one aromatic or heteroaromatic ring include, but are not limited to, phenyl, napthyl, chromenyl, pyridyl, indolyl, pyrimidinyl, pyridizinyl, pyrazinyl, imidazolyl, oxazolyl, furanyl, thiophenyl, thiazolyl, triazolyl, tetrazolyl, isoxazolyl, quinolinyl, pyrrolyl, pyrazolyl, dibenzo[b,d]thiophenyl, 2H-benzo[b][1,4]dioxepinyl, 2,3-dihydrobenzo[b][1,4]dioxinyl, benzo[d][1,3]dioxolyl, 2,3-dihydrobenzofuranyl, 4H-chromenyl, isoquinolinyl, quinazolinyl, quinoxalinyl, thienyl, isoindolyl, 9H-fluorenyl, and 5,6,7,8-tetrahydroisoquinoline.

“Heterocycloalkyl” means a saturated cyclic group containing from 1 to about 3 heteroatoms chosen from N, O, and S, with remaining ring atoms being carbon. Heterocycloalkyl groups have from 3 to about 8 ring atoms, and more typically have from 5 to 7 ring atoms. Examples of heterocycloalkyl groups include morpholinyl, piperazinyl, piperidinyl, and pyrrolidinyl groups. A nitrogen in a heterocycloalkyl group may optionally be quaternized.

“Haloalkyl” indicates both branched and straight-chain alkyl groups having the specified number of carbon atoms, substituted with 1 or more halogen atoms, generally up to the maximum allowable number of halogen atoms. Examples of haloalkyl include, but are not limited to, trifluoromethyl, difluoromethyl, 2-fluoroethyl, and penta-fluoroethyl.

“Haloalkoxy” indicates a haloalkyl group as defined above attached through an oxygen bridge (oxygen of an alcohol radical).

“Halo” or “halogen” as used herein refers to fluoro, chloro, bromo, or iodo.

“Pharmaceutical compositions” are compositions comprising at least one active agent, such as a compound or salt of Formula I (or II), and at least one other substance, such as a carrier, excipient, or diluent. Pharmaceutical compositions meet the U.S. FDA's GMP (good manufacturing practice) standards for human or non-human drugs.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)—COOH where n is 0-4, and the like. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions of the invention refers to a diluent, excipient, or vehicle with which an active compound is administered.

A “pharmaceutically acceptable carrier” means a carrier that is useful in preparing a pharmaceutical composition that is generally safe, non-toxic and neither biologically nor otherwise undesirable, and includes an excipient that is acceptable for veterinary use as well as human pharmaceutical use.

The phrase “optionally substituted” indicates that such groups may either be unsubstituted or substituted at one or more of any of the available positions, typically 1, 2, 3, or 4 positions, by one or more suitable groups such as those disclosed herein.

Suitable groups that may be present on an “optionally substituted” position include, but are not limited to, e.g., halogen, cyano, hydroxyl, amino, nitro, oxo, azido, alkanoyl (such as a C₂-C₆ alkanoyl group such as acyl or the like); carboxamido; alkylcarboxamide; alkyl groups, alkoxy groups, alkylthio groups including those having one or more thioether linkages, alkylsulfinyl groups including those having one or more sulfinyl linkages, alkylsulfonyl groups including those having one or more sulfonyl linkages, mono- and di-aminoalkyl groups including groups having one or more N atoms, all of the foregoing optional alkyl substituents may have one or more methylene group replaced by an oxygen or —NH—, and have from about 1 to about 8, from about 1 to about 6, or from 1 to about 4 carbon atoms, cycloalkyl; phenyl; phenylalkyl with benzyl being an exemplary phenylalkyl group, phenylalkoxy with benzyloxy being an exemplary phenylalkoxy group; a saturated, unsaturated, or aromatic heterocyclic groups having 1 ring and one or more N, O or S atoms, e.g. pyridyl, pyrazinyl, pyrimidinyl, furanyl, pyrrolyl, thienyl, thiazolyl, triazinyl, oxazolyl, isoxazolyl, imidazolyl, tetrahydrofuranyl, tetrahydropyranyl, piperidinyl, morpholinyl, piperazinyl, and pyrrolidinyl. Any such groups having additional positions available for substitution may be further substituted, e.g with substituents independently chosen from, e.g., amino, hydroxy, alkyl, alkoxy, halogen, haloalkyl, haloalkoxy, and mono- and di-alkylamino.

A “patient” is a human or non-human animal in need of medical treatment. In some embodiments the patient is a human patient.

“Providing” means giving, administering, selling, distributing, transferring (for profit or not), manufacturing, compounding, or dispensing.

“Providing a compound of Formula I (or II) with at least one additional active agent” means the compound of Formula I (or II) and the additional active agent(s) are provided simultaneously in a single dosage form, provided concomitantly in separate dosage forms, or provided in separate dosage forms for administration separated by some amount of time that is within the time in which both the compound of Formula I (or II) and the at least one additional active agent are within the blood stream of a patient. The compound of Formula I (or II) and the additional active agent need not be prescribed for a patient by the same medical care worker. The additional active agent or agents need not require a prescription. Administration of the compound of Formula I (or II) or the at least one additional active agent can occur via any appropriate route, for example, oral tablets, oral capsules, oral liquids, inhalation, injection, suppositories or topical contact.

“Treatment,” as used herein includes providing a compound as described herein and at least one additional active agent sufficient to: (a) prevent a disease or a symptom of a disease from occurring in a patient who may be predisposed to the disease but has not yet been diagnosed as having it (e.g. a patient identified as having a genetic defect in the SMN1 or SMN2 gene or identified as having abnormally low levels of full length SMN protein but not yet exhibiting symptoms of SMA); (b) inhibiting the disease, i.e. arresting its development or slowing its progression; and (c) relieving the disease, i.e., causing regression of the disease. “Treating” and “treatment” also means providing a therapeutically effective amount of a compound of Formula I (or II) and at least one additional active agent to a patient having or susceptible SMA.

A “therapeutically effective amount” of a pharmaceutical combination of this invention means an amount effective, when administered to a patient, to provide a therapeutic benefit such as an amelioration of symptoms, e.g., an amount effective to decrease the symptoms of a SMA or to slow the progression of the disease. A therapeutically effective amount is also an amount sufficient to significantly increase the copy number of SMN2 or to significantly increase detectable level of full length SMN protein.

A significant increase or reduction in the SMN2 copy number or detectable level of full length SMN protein is an increase that is statistically significant in a standard parametric test of statistical significance such as Student's T-test, where p<0.05.

### Chemical Description

As disclosed in the SUMMARY section above, compounds and pharmaceutically acceptable salts of Formula I and Formula II are provided herein.

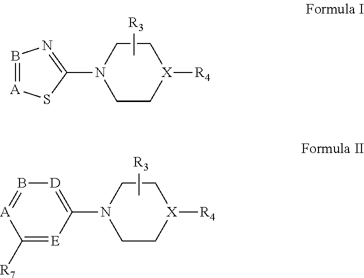

Compounds of Formula III, IV, and V are provided herein.

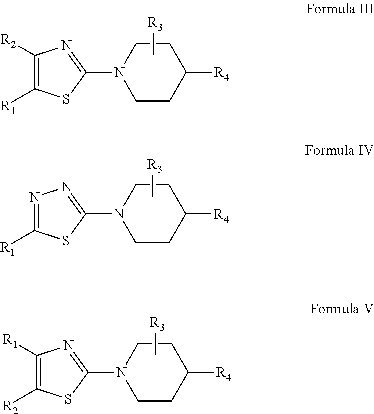

These compounds are subformulae of Formula I in which:

X is CH, A is CR₁ and B is CR₂ (Formula III);

X is CH, A is CR₁ and B is N (Formula IV); and

X is CH, A is CR₂ and B is CRi (Formula V).

The variables in Formula III, IV, and V may carry the definitions set forth for Formula I or any of the definitions set forth below.

Compounds of Formula VI, VII, VIII, IX, and X are also provided herein.

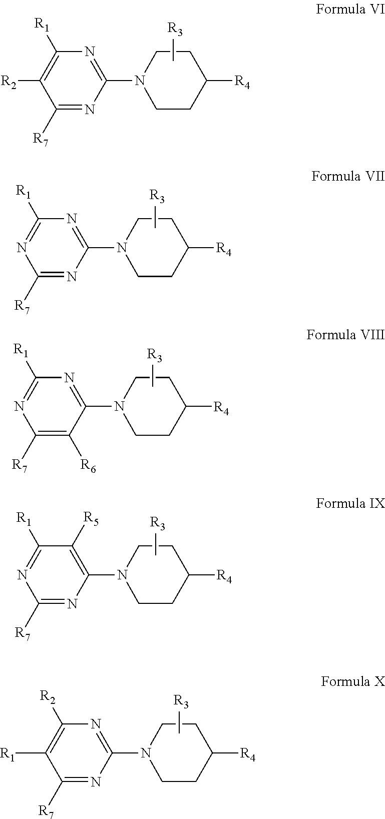

Compounds of subformulae VI to X are subformulae of Formula II in which:

### X is CH, A is R₂, B is R₁, D is N, and E is N (Formula VI);

### X is CH, A is N, B is R₁, D is N, and E is N (Formula VII);

### X is CH, A is N, B is R₁, D is N, and E is CR₆ (Formula VIII);

### X is CH, A is N, B is R₁, D is CR₅, and E is N (Formula IX); and

### X is CH, A is R₁, B is R₂, D is N, and E is N (Formula X).

The variables in Formula VI to X may carry the definitions set forth for Formula II or any of the definitions set forth below.

Further provided herein are compounds and salts of Formula I and Formula II, and the subformulae thereof, in which the variables, e.g., A, B, D, E, X, R₃, R₄, and R₇, carry any of the following definitions. An of the variable definitions provided herein can be combined with any of the other variable definitions provided herein so long as a stable compound of Formula I or Formula II results.

### The R₁ Variable

Compounds and salts of Formula I and Formula II in which R₁ carries any of the following definitions are provided herein.

R₁ is

(i) a 5-membered heteroaryl group containing 1, 2 or 3 heteroatoms chosen from N, O, and S and not more than one O or S heteroatoms:

(ii) phenyl, naphthyl, or a 6-membered heteroaryl group containing one or two nitrogen atoms;

(iii) a phenyl group fused to a 5, 6, or 7-membered heterocycloalkyl group containing one or two heteroatoms independently chosen from N, O, and S; or

(iv) a benzofuranyl, indolyl, 9H-fluorenyl, or dibenzo[b,d]thiophenyl group.

Each of which (i) and (ii) is substituted with 0 to 3 substituents independently chosen from:

(a) halogen, hydroxy, amino, cyano, nitro, oxo, —(C═O)NH₂, C₁-C₆alkyl, C₁-C₆alkoxy, C₂-C₄alkanoyl, C₁-C₄alkylthio, C₁-C₄alkylsulfonyl, mono- and di-(C₁-C₄alkylamino)C₀-C₂alkyl, mono- and di-(C₁-C₄alkyl)carboxamide, mono- and di-(C₁-C₄alkyl)sulfonamide, C₁-C₂haloalkyl, and C₁-C₂haloalkoxy, and

(b) (C₃-C₆cycloalkyl)C₀-C₂alkyl, (heterocycloalkyl)C₀-C₂alkyl; (phenyl)C₀-C₂alkyl, (phenyl)C₀-C₂alkoxy, and thienyl, each of which (b) is substituted with 0 to 2 substituents independently chosen from halogen, C₁-C₂alkyl, and C₁-C₂alkoxy.

Each of which (iii) and (iv) is substituted with 0 to 4 substituents independently chosen from halogen, C₁-C₂alkyl, C₁-C₂alkyl, mono- and di-(C₁-C₂alkyl)amino, trifluoromethyl, and trifluoromethoxy.

R₁ is

(i) a 5-membered heteroaryl group containing 1, 2 or 3 heteroatoms chosen from N, O, and S and not more than one O or S heteroatoms:

(ii) phenyl, naphthyl, or a pyridyl group containing one or two nitrogen atoms;

(iii) a phenyl group fused to a 5, 6, or 7-membered heterocycloalkyl group containing one or two heteroatoms independently chosen from N, O, and S chosen from 2H-benzo[b][1,4]dioxepinyl, 2,3-dihydrobenzo[b][1,4]dioxinyl, benzo[d][1,3]dioxolyl, 2,3-dihydrobenzofuranyl, 4H-chromenyl;

(iv) a benzofuranyl, indolyl, 9H-fluorenyl, or dibenzo[b,d]thiophenyl group.

Each of which (i) and (ii) is substituted with 0 to 3 substituents independently chosen from: (a) halogen, hydroxy, amino, cyano, nitro, oxo, —(C═O)NH₂, C₁-C₄alkyl, C₁-C₄alkoxy, C₂-C₄alkanoyl, C₁-C₂alkylthio, C₁-C₂alkylsulfonyl, mono- and di-(C₁-C₄alkylamino)C₀-C₂alkyl, mono- and di-(C₁-C₂alkyl)carboxamide, trifluoromethyl, trifluoromethoxy, CF₃S— and (b) piperidinyl, morpholinyl, piperazinyl, pyrrolidinyl, (phenyl)C₀-C₂alkyl, (phenyl)C₀-C₂alkoxy, and thienyl, each of which (b) is substituted with 0 to 2 substituents independently chosen from halogen, C₁-C₂alkyl, and C₁-C₂alkoxy.

Each of which (iii) and (iv) is substituted with 0 to 2 substituents independently chosen from halogen, methyl, and methoxy.

R₁ is phenyl or pyridyl, each of which is substituted with 1 to 3 substituents independently chosen from

(a) halogen, hydroxy, amino, cyano, nitro, —(C═O)NH₂, C₁-C₄alkyl, C₁-C₄alkoxy, C₂-C₄alkanoyl, C₁-C₂alkylthio, C₁-C₂alkylsulfonyl, mono- and di-(C₁-C₄alkylamino), mono- and di-(C₁-C₂alkyl)carboxamide, trifluoromethyl, trifluoromethoxy, CF₃S—, and

(b) piperidinyl, morpholinyl, piperazinyl, pyrrolidinyl, (phenyl)C₀-C₂alkyl, (phenyl)C₀-C₂alkoxy, and thienyl, each of which is substituted with 0 to 2 substitutents independently chosen from halogen, C₁C₂alkyl, and C₁-C₂alkoxy.

R₁ is phenyl or pyridyl, each of which is substituted with 1 to 3 substituents, wherein

0 to 3 substituents are independently chosen from bromo, chloro, fluoro, nitro, acetyl, cyano, C₁-C₄alkyl, C₁-C₄alkoxy, dimethylamino, dimethylcarboxamide, methylthio, trifluoromethyl, and trifluoromethoxy, and 0 or 1 substituents are chosen from piperidinyl, morpholinyl, pyrrolidinyl, phenoxy, and thienyl.

R₁ is a group of the formula

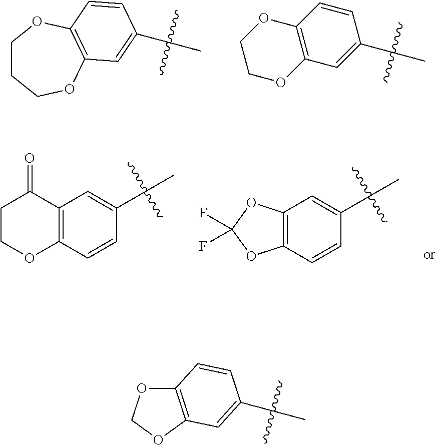 [SMILES: *c1ccc2c(c1)OCCCO2 ; *c1ccc2c(c1)OCCO2 ; *c1ccc2c(c1)C(=O)CCO2 ; *c1ccc2c(c1)OC(F)(F)O2 ; *c1ccc2c(c1)OCO2]

each of which is substituted with 0 to 2 substituents independently chosen from halogen, methyl, and methoxy.

R₁ is a benzofuranyl, indolyl, 9H-fluorenyl, or dibenzo[b,d]thiophenyl group;

each of which is substituted with 0 to 2 substituents independently chosen from halogen, methyl, and methoxy.

In certain embodiments R₁ is not unsubstituted phenyl, 4-bromo-phenyl, 4-methoxy-phenyl, 4-chloro-phenyl, 4-fluoro-phenyl, 2,5-dimethoxy-phenyl, 2,4-dichloro-phenyl, 1-naphthyl, or 4-nitro-phenyl.

In certain embodiments R₁ is not unsubstituted phenyl, naphthyl, or phenyl substituted only with halogen, methoxy, or nitro.

### The R₂ Variable

Compounds and salts of Formula I and Formula II in which R₂ or R₃ carries any of the following definitions are provided herein.

R₂ is hydrogen and R₃ is hydrogen at each occurrence.

R₂ is fluoro and R₃ is hydrogen at each occurrence.

### The R₄ Variable

Compounds and salts of Formula I and Formula II in which R₄ carries any of the following definitions are provided herein.

R₄ is cyano, amino, (C═O)R₁₀, —(C═O)NR₁₀R₁₁, (C═O)OR₁₀, —NR₁₂(C═O)R₁₀, NR₁₂(C═O)OR₁₀, imidazolyl, tetrazolyl; wherein R₁₀ and R₁₁ are independently chosen from hydrogen, C₁-C₄alkyl, morpholinyl, piperazinyl, and (phenyl)C₀-C₄alkyl; and R₁₂ is hydrogen or methyl.

R₄ is —(C═O)OH, —(C═O)NH₂, —(C═O)C₁-C₂alkyl, —(C═O)OC₁-C₂alkyl, —(C═O)NH(phenyl) or imidazolyl.

R₄ is —(C═O)OH or —(C═O)NH₂.

### Pharmaceutical Preparations

Aryl substituted thiazol-2-yl-piperidines and related compounds described herein can be administered as the neat chemical, but are specifically administered as a pharmaceutical composition, for example a pharmaceutical formulation comprising a Aryl substituted thiazol-2-yl-piperidines or related compound of Formula I or II or a or pharmaceutically acceptable salt thereof, together with at least one pharmaceutically acceptable carrier.

The compounds of Formula I and II may be administered orally, topically, parenterally, by inhalation or spray, sublingually, transdermally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, an injectable fluid, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorings, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active and/or inactive agents may be included in the pharmaceutical compositions, provided that such agents do not substantially interfere with the activity of the Aryl substituted thiazol-2-yl-piperidines and related compounds used in the pharmaceutical compositions. The optional active is an additional active agent that is not a compound or salt of Formula I or Formula II.

The pharmaceutical compositions can be formulated for oral administration. These compositions contain between 0.1 and 99 weight % (wt. %) of an aryl substituted thiazol-2-yl-piperidine or related compounds and usually at least about 5 wt. % of a quinazolin-4-amine derivative. Some embodiments contain from about 25 wt. % to about 50 wt. % or from about 5 wt. % to about 75 wt. % of the aryl substituted thiazol-2-yl-piperidine or related compound.

### Packaged Formulations

Methods provided herein include providing a compound or salt of Formula I or II in a container together with instructions for using the composition to treat a patient suffering from or susceptible to SMA.

The invention includes packaged pharmaceutical combinations. Such packaged combinations include a compound of Formula I or II in a container. The container may additionally include instructions for using the combination to treat or prevent SMA in a patient.

The packaged pharmaceutical combination may include one or more additional active agents.

### Methods of Treatment

The compounds of Formula I and II and the pharmaceutically acceptable salts thereof, as well as pharmaceutical compositions comprising the compounds, are useful for treating a SMA in a patient. An effective amount of a pharmaceutical composition comprising a compound of Formula I or II may be an amount sufficient to a) prevent a disease or a symptom of a disease from occurring in a patient who may be predisposed to the disease but has not yet been diagnosed as having it (e.g. a patient identified as having a genetic defect in the SMN1 or SMN2 gene or identified as having abnormally low levels of full length SMN protein but not yet exhibiting symptoms of SMA); (b) inhibiting the disease, i.e. arresting its development or slowing its progression; and (c) relieving the disease, i.e., causing regression of the disease. “Treating” and “treatment” also means providing a therapeutically effective amount of a compound of Formula I (or II) and at least one additional active agent to a patient having or susceptible SMA.

An effective amount of a compound or pharmaceutical composition described herein will also provide a sufficient concentration of a compound of Formula I or II when administered to a patient. A sufficient concentration is a concentration of the compound in the patient's body necessary to prevent SMA symptoms, relieve SMA symptoms, or slow the progression of the disorder. Such an amount may be ascertained experimentally, for example by assaying blood concentration of the compound, or theoretically, by calculating bioavailability. The amount of an active agent sufficient to modulate SMN levels in vivo may be determined in vitro with a conventional assay for SMN protein.

Methods of treatment include providing certain dosage amounts of a compound of Formula I or II to a patient. Dosage levels of each compound of from about 0.1 mg to about 140 mg per kilogram of body weight per day are useful in the treatment of the above-indicated conditions (about 0.5 mg to about 7 g per patient per day). The amount of compound that may be combined with the carrier materials to produce a single dosage form will vary depending upon the patient treated and the particular mode of administration. Dosage unit forms will generally contain between from about 1 mg to about 500 mg of each active compound. In certain embodiments 25 mg to 500 mg, or 25 mg to 200 mg of a compound of Formula I or II are provided daily to a patient. Frequency of dosage may also vary depending on the compound used and the particular disease treated. However, for treatment of SMA a dosage regimen of 4 times daily or less can be used and in certain embodiments a dosage regimen of 1 or 2 times daily is used.

The compounds of Formula I may be used to treat SMA. It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disease undergoing therapy.

### EXAMPLES

### Abbreviations

DIPEA diisopropylethylamine

DMA N,N-dimethylacetamide

DMAP 4-dimethylaminopyridine

DMF dimethylformamide

DMSO dimethylsulfoxide

EtOAc ethyl acetate

EtOH ethanol

EDC N-(3-Dimethylaminopropyl)-N′-ethylcarbodiimide hydrochloride

HOBt 1-hydroxybenzotriazole

KOCN potassium cyanate

MCPBA meta-chloroperoxybenzoic acid

MeOH methanol

Si-THIOL 3-mercaptopropyl silica gel

TEA triethylamine

TFA trifluoroacetic acid

### General Methods

Unless otherwise stated, all reactions were carried out under an atmosphere of dry argon or nitrogen in dried glassware. Indicated reaction temperatures refer to those of the reaction bath, while room temperature (rt) is noted as 25° C. All solvents were of anhydrous quality purchased from Aldrich Chemical Co. and used as received. Commercially available starting materials and reagents were purchased from Aldrich, TCI, and Acros and were used as received. Analytical thin layer chromatography (TLC) was performed with Sigma Aldrich TLC plates (5×20 cm, 60 Å, 250 μm). Visualization was accomplished by irradiation under a 254 nm UV lamp. Chromatography on silica gel was performed using forced flow (liquid) of the indicated solvent system on Biotage KPSil pre-packed cartridges and using the Biotage SP-1 automated chromatography system. ¹H NMR spectra were recorded on a Varian Inova 400 MHz spectrometer. Chemical shifts are reported in ppm with the solvent resonance as the internal standard (CDCl₃ 7.27 ppm, DMSO-d₆ 2.50 ppm, for ¹H NMR). Data are reported as follows: chemical shift, multiplicity (s=singlet, d=doublet, t=triplet, q=quartet, sep=septet, quin=quintet, br=broad, m=multiplet), coupling constants, and number of protons. Low resolution mass spectra (electrospray ionization) were acquired on an Agilent Technologies 6130 quadrupole spectrometer coupled to an Agilent Technologies 1200 series HPLC. The HPLC retention time were recorded through standard gradient 4% to 100% acetonitrile (0.05% TFA) over 7 minutes using Luna C₁₈ 3 micron 3×75 mm column with a flow rate of 0.800 mL/min. High resolution mass spectral data was collected in-house using and Agilent 6210 time-of-flight mass spectrometer, also coupled to an Agilent Technologies 1200 series HPLC system.

### General Protocol A.

A mixture of 1-(4-bromothiazol-2-yl)piperidine-4-carboxamide (0.100 mmol), boronic acid (0.200 mmol) and tetrakis(triphenylphosphine)palladium (5.0011=1) in DMF (1.50 mL) or CH₃CN (1.50 mL) and 2.0 M Na₂CO₃ aqueous solution (0.50 mL) was heated in 1.1 W at 100° C. for 30 min. The reaction was cooled to room temperature, added a small portion of Si-THIOL to get rid of palladium. The mixture was filtered through a frit to give light yellow solution. The crude material was purified by preparative HPLC under acidic or basic condition to give the final product.

### General Protocol B.

A mixture of carboxylic acid (0.082 mmol), EDC (0.082 mmol), HOBt (0.082 mmol), DMAP (10.0 mg, 0.082 mmol) and amine (0.163 mml) in DMF (1.50 mL) was heated in μW at 100° C. for 1.5 h. The reaction mixture was purified by preparative HPLC under acidic condition to give the final product as a TFA salt.

### General Protocol C.

To a solution of 4-(4-bromophenyl)-2-(piperazin-1-yl)thiazole (0.093 mmol), Et₃N (0.139 mmol) in CH₂Cl₂ (2.00 mL) was treated at 0° C. with carboxylic chloride or sulfonyl chloride (0.111 mmol). The reaction mixture was allowed to warm to room temperature and stirred for 1 h. The crude mixture was purified by preparative HPLC under acidic or basic conditions to give the final product.

### Example 1

### 1-(4-Bromothiazol-2-yl)Piperidine-4-Carboxamide (XJB01-018)

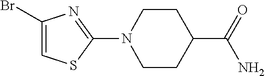 [SMILES: NC(=O)C1CCN(c2nc(Br)cs2)CC1]

A mixture of 2,4-dibromothiazole (2.06 g, 8.48 mmol), piperidine-4-carboxamide (1.30 g, 10.1 mmol) and TEA (2.50 mL) in ethanol (5.00 mL) was heated in μW at 100° C. for 1 hour. The reaction mixture was cooled to room temperature, diluted with water and extracted with methanol and dichloromethane. The organic layer was separated, dried with Na₂SO₄, and concentrated as light brown solid. The crude mixture was purified by Biotage with 0-10% MeOH in CH₂Cl₂ with 1% TEA to give 2.08 g (85%) product as a white solid: ¹H NMR (400 MHz, DMSO-d₆) δ ppm 7.31 (br. s., 1H), 6.85 (s, 1H), 6.82 (br. s., 1H), 3.77-3.90 (m, 2H), 3.03 (td, J=12.5, 2.8 Hz, 2H), 2.29-2.39 (m, 1H), 1.78 (dd, J=13.5, 3.1 Hz, 2H), 1.50-1.63 (m, 2H); LCMS RT=4.134 min, m/z 289.9 [M+H⁺].

### Example 2

### 1-(5-Bromothiazol-2-yl)Piperidine-4-Carboxamide (XJB02-010)

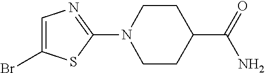 [SMILES: NC(=O)C1CCN(c2ncc(Br)s2)CC1]

A mixture of 2,5-dibromothiazole (1.42 g, 5.85 mmol), piperidine-4-carboxamide (1.50 g, 11.7 mmol) in EtOH (5.00 mL) and TEA (2.50 mL) was heated in μW at 120° C. for 2 hours. The reaction mixture was cooled to room temperature, diluted with water and extracted with EtOAc and dichloromethane. The organic layer was separated, dried Na₂SO₄, and concentrated as light yellow solid. The crude mixture was purified with Biotage with 0-25% MeOH in CH₂Cl₂ to give 1.35 g (80%) product as a white solid: ¹H NMR (400 MHz, DMSO-d₆) δ ppm 7.30 (br. s., 1H), 7.18 (s, 1H), 6.81 (br. s., 1H), 3.80 (dt, J=12.7, 3.3 Hz, 2H), 3.02 (td, J=12.6, 3.0 Hz, 2H), 2.34 (tt, J=11.5, 3.8 Hz, 1H), 1.77 (dd, J=14.0, 3.6 Hz, 2H), 1.49-1.63 (m, 2H); LCMS RT=3.746 min, m/z 290.01 [M+H⁺].

### Example 3

### 1-(5-Bromo-1,3,4-Thiadiazol-2-yl)Piperidine-4-Carboxamide (XJB03-058)

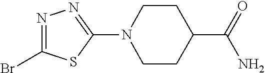 [SMILES: NC(=O)C1CCN(c2nnc(Br)s2)CC1]

A mixture of 2,5-dibromo-1,3,4-thiadiazole (500 mg, 2.05 mmol), piperidine-4-carboxamide (263 mg, 2.05 mmol) in EtOH (2.00 mL) and TEA (1.00 mL) was heated μW at 120° C. for 2 h. The reaction mixture was cooled to room temperature, diluted with water and extracted with methanol and dichloromethane. The organic layer was separated, dried Na₂SO₄, and concentrated as light brown solid. The crude mixture was purified with Biotage using 0-20% MeOH in CH₂Cl₂ followed by cartridge filtration to give 525 mg (88%) product as a yellow solid: ¹H NMR (400 MHz, DMSO-d₆) δ ppm 7.31 (br. s., 1H), 6.84 (br. s., 1H), 3.79 (dt, J=12.9, 3.3 Hz, 2H), 3.16 (td, J=12.6, 2.9 Hz, 2H), 2.36 (tt, J=11.4, 3.8 Hz, 1H), 1.79 (dd, J=13.7, 3.5 Hz, 2H), 1.42-1.68 (m, 2H); LCMS RT=3.483 min, m/z 290.9 [M+H⁺]; HRMS (ESI) m/z calcd for C₈H₁₂⁷⁹BrN₄OS [M+H⁺] 290.9915.

### Example 4

### Ethyl 1-(4-Bromothiazol-2-yl)Piperidine-4-Carboxylate (XJB01-034)

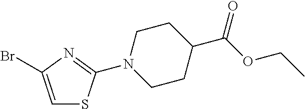 [SMILES: CCOC(=O)C1CCN(c2nc(Br)cs2)CC1]

A mixture of 2,4-dibromothiazole (500 mg, 2.06 mmol), ethyl piperidine-4-carboxylate (380 mg, 2.42 mmol) and TEA (2.50 mL) in 1,4-dioxane (5.00 mL) was heated in μW at 100° C. for 1 hour. The reaction mixture was cooled to room temperature, filtered, and concentrated as light brown solid. The crude mixture was purified by Biotage with 0-50% EtOAc in hexanes to give 510 mg (79%) product as a light yellow oil: ¹H NMR (400 MHz, CHLOROFORM-d) δ ppm 6.41 (s, 1H), 4.17 (q, J=7.0 Hz, 2H), 3.92 (ddd, J=13.5, 4.3, 4.1 Hz, 2H), 3.12 (ddd, J=13.2, 11.2, 3.3 Hz, 2H), 2.53 (tt, J=11.0, 3.9 Hz, 1H), 1.94-2.12 (m, 2H), 1.70-1.90 (m, 2H), 1.28 (t, J=7.0 Hz, 3H); LCMS RT=6.110 min, m/z 319.0 [M+H⁺]; HRMS (ESI) m/z calcd for C₁₁H₁₆⁷⁹BrN₂O₂S [M+H⁺] 319.0116.

### Example 5

### 1-(4-Bromothiazol-2-yl)Piperidine-4-Carbonitrile (XJB02-030)

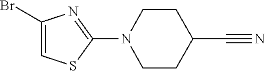 [SMILES: N#CC1CCN(c2nc(Br)cs2)CC1]

A mixture of 2,4-dibromothiazole (500 mg, 2.06 mmol), piperidine-4-carbonitrile (272 mg, 2.47 mmol) in 1,4-dioxane (5.00 mL) and TEA (2.50 mL) was heated in 1.1 W at 100° C. for 1 hour. The reaction mixture was cooled to room temperature, diluted with water and extracted with EtOAc and dichloromethane. The organic layer was separated, dried Na₂SO₄, and concentrated as light yellow solid. The crude mixture was purified with Biotage with 0-10% MeOH in CH₂Cl₂ to give 354 mg (63%) product as a white solid: ¹H NMR (400 MHz, CHLOROFORM-d) δ ppm 6.46 (s, 1H), 3.71 (ddd, J=13.4, 7.5, 3.9 Hz, 2H), 3.49 (ddd, J=13.5, 7.3, 3.8 Hz, 2H), 2.77-3.02 (m, 1H), 1.81-2.18 (m, 4H); LCMS RT=5.271 min, m/z 271.9 [M+H⁺]; HRMS (ESI) m/z calcd for C₉H₁₁⁷⁹BrN₃S [M+H⁺] 271.985.

### Example 6

### Tert-Butyl 1-(4-Bromothiazol-2-yl)Piperidin-4-Ylcarbamate (XJB02-032)

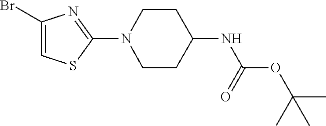 [SMILES: CC(C)(C)OC(=O)NC1CCN(c2nc(Br)cs2)CC1]

A mixture of 2,4-dibromothiazole (500 mg, 2.06 mmol), tert-butyl piperidin-4-ylcarbamate (495 mg, 2.47 mmol) in 1,4-dioxane (5.00 mL) and TEA (2.50 mL) was heated in μW at 100° C. for 1 h and at 120° C. for another 3 h. The reaction mixture was cooled to room temperature, diluted with water and extracted with EtOAc and dichloromethane. The organic layer was separated, dried Na₂SO₄, and concentrated as light yellow solid. The crude mixture was purified with Biotage with 0-10% MeOH in CH₂Cl₂ to give 654 mg (88%) product as a white solid: ¹H NMR (400 MHz, CHLOROFORM-d) δ ppm 6.41 (s, 1H), 4.46 (br. s., 1H), 3.92 (dt, J=13.4, 3.3 Hz, 2H), 3.70 (br. s., 1H), 3.13 (ddd, J=13.4, 11.6, 3.1 Hz, 2H), 1.88-2.19 (m, 2H), 1.40-1.54 (m, 11H); LCMS RT=6.137 min, m/z 362.0 [M+H⁺]; HRMS (ESI) m/z calcd for C₁₃H₂₁⁷⁹BrN₃O₂S [M+H⁺] 362.0538.

### Example 7

### 4-Bromo-2-(Piperazin-1-yl)Thiazole (XJB02-056)

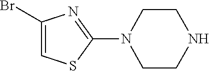 [SMILES: Brc1csc(N2CCNCC2)n1]

A mixture of 2,4-dibromothiazole (1.00 g, 4.12 mmol), piperazine (426 mg, 4.94 mmol) in 1,4-dioxane (5.00 mL) and TEA (2.50 mL) was heated in μW at 100° C. for 1 hour. The reaction mixture was cooled to room temperature, diluted with water and extracted with EtOAc and dichloromethane. The organic layer was separated, dried Na₂SO₄, and concentrated as light yellow solid. The crude product was purified with Biotage using 0-10% MeOH in CH₂Cl₂ to give 581 mg (57%) product as a white solid: ¹H NMR (400 MHz, CHLOROFORM-d) δ ppm 6.43 (s, 1H), 3.39-3.58 (m, 4H), 2.83-3.06 (m, 4H), 1.80 (br. s., 1H); LCMS RT=3.038 min, m/z 247.9 [M+H⁺]; HRMS (ESI) m/z calcd for C₇H₁₁⁷⁹BrN₃S [M+H⁺] 247.9857.

### Example 8

### Ethyl 1-(5-Bromothiazol-2-yl)Piperidine-4-Carboxylate (XJB03-098)

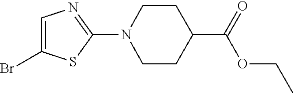 [SMILES: CCOC(=O)C1CCN(c2ncc(Br)s2)CC1]

A mixture of 2,5-dibromothiazole (500 mg, 2.06 mmol), ethyl piperidine-4-carboxylate (485 mg, 3.09 mmol) and TEA (3.00 mL) in ethanol (6.00 mL) was heated in μW at 120° C. for 2 hours. The reaction mixture was cooled to room temperature, diluted with water and extracted with EtOAc and dichloromethane. The organic layer was separated, dried Na₂SO₄, and concentrated as light yellow solid. The crude mixture was purified by Biotage with 0-50% EtOAc in hexanes to give 454 mg (69%) product as a light yellow oil: ¹H NMR (400 MHz, DMSO-d₆) δ ppm 7.19 (s, 1H), 4.08 (q, J=7.2 Hz, 2H), 3.75 (ddd, J=13.2, 3.7, 3.5 Hz, 2H), 3.10 (ddd, J=13.0, 11.5, 3.0 Hz, 2H), 2.61 (tt, J=11.1, 3.8 Hz, 1H), 1.79-1.97 (m, 2H), 1.46-1.69 (m, 2H), 1.19 (t, J=7.1 Hz, 3H); LCMS RT=5.848 min, m/z 319.0 [M+H⁺]; HRMS (ESI) m/z calcd for C₁₁H₁₆⁷⁹BrN₂O₂S [M+H⁺] 319.0116.

### Example 9

### 1-(5-Bromo-1,3,4-Thiadiazol-2-yl)Piperidine-4-Carboxamide (XJB03-079)

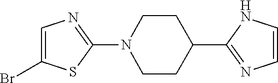 [SMILES: Brc1cnc(N2CCC(c3ncc[nH]3)CC2)s1]

A mixture of 2,5-dibromothiazole (500 mg, 2.06 mmol), 4-(1H-imidazol-2-yl)piperidine, HCl salt (461 mg, 2.06 mmol) in EtOH (3.00 mL) and TEA (1.50 mL) was heated μW at 160° C. for 2 h. The reaction mixture was cooled to room temperature, diluted with water and extracted with methanol and dichloromethane. The organic layer was separated, dried Na₂SO₄, and concentrated as light brown solid. The crude mixture was purified with Biotage using 0-25% MeOH in CH₂Cl₂ to give 407 mg (63%) product as a white solid which was used directly in the next reaction without further purification.

### Example 10

### 1-(1-(5-Bromothiazol-2-yl)Piperidin-4-yl)Ethanone (XJB03-087)

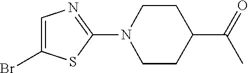

A mixture of 2,5-dibromothiazole (500 mg, 2.06 mmol), 1-(piperidin-4-yl)ethanone, TFA salt (645 mg, 2.06 mmol) and TEA (4.00 mL) in ethanol (8.00 mL) was heated in μW at 160° C. for 2 hours. The reaction mixture was cooled to room temperature and concentrated as light yellow solid. The crude mixture was purified by Biotage with 0-100% EtOAc in hexanes to give 239 mg (40%) product as a yellow solid: ¹H NMR (400 MHz, DMSO-d₆) δ ppm 7.18 (s, 1H), 3.79 (dt, J=12.8, 3.4 Hz, 2H), 2.94-3.15 (m, 2H), 2.63 (tt, J=11.5, 3.8 Hz, 1H), 2.14 (s, 3H), 1.79-1.96 (m, 2H), 1.35-1.57 (m, 2H); LCMS RT=4.889 min, m/z 289.0 [M+H⁺]; HRMS (ESI) m/z calcd for C₁₀H₁₄⁷⁹BrN₂OS [M+H⁺] 289.0010.

### Example 11

### 1-(5-Bromopyrimidin-2-yl)Piperidine-4-Carboxamide (XJB02-011)

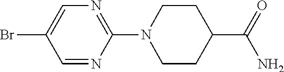 [SMILES: NC(=O)C1CCN(c2ncc(Br)cn2)CC1]

A mixture of 5-bromo-2-chloropyrimidine (500 mg, 1.55 mmol), piperidine-4-carboxamide (239 mg, 1.86 mmol) in 1,4-dioxane (2.00 mL) and TEA (1.00 mL) was heated in μW at 100° C. for 2 hour. The reaction mixture was cooled to room temperature, diluted with water and extracted with methanol and dichloromethane. The organic layer was separated, dried Na₂SO₄, and concentrated as light yellow solid. The crude mixture was purified with Biotage with 0-10% MeOH in CH₂Cl₂ to give 251 mg (57%) product as a white solid: ¹H NMR (400 MHz, DMSO-d₆) δ ppm 8.43 (s, 2H), 7.28 (br. s., 1H), 6.78 (br. s., 1H), 4.54 (dt, J=13.2, 2.9 Hz, 2H), 2.80-3.01 (m, 2H), 2.36 (tt, J=11.4, 3.8 Hz, 1H), 1.75 (dd, J=13.2, 3.2 Hz, 2H), 1.42 (qd, J=12.5, 4.1 Hz, 2H); LCMS RT=4.263 min, m/z 285.0 [M+H⁺]; HRMS (ESI) m/z calcd for C₁₀H₁₄⁷⁹BrN₄O [M+H⁺] 285.0351.

### Example 12

### 1-(4-Chloropyrimidin-2-yl)Piperidine-4-Carboxamide (XJB02-012-1)

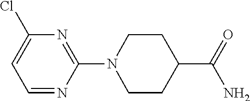 [SMILES: NC(=O)C1CCN(c2nccc(Cl)n2)CC1]

A mixture of 2,4-dichloropyrimidine (1.00 g, 6.71 mmol), piperidine-4-carboxamide (1.03 g, 8.05 mmol) in 1,4-dioxane (5.00 mL) and TEA (2.50 mL) was heated in μW at 100° C. for 1 hour. The reaction mixture was cooled to room temperature, diluted with water and extracted with methanol and dichloromethane. The organic layer was separated, dried Na₂SO₄, and concentrated as light brown solid. The crude mixture was purified with FCC with 0-10% MeOH in CH₂Cl₂ with 1% TEA to give 274 mg (17%) XJB02-012-1, less polar, as a white solid and 588 mg (36%) XJB02-012-2, more polar, as a white solid. XJB02-012-1: ¹H NMR (400 MHz, DMSO-d₆) δ ppm 8.30 (d, J=5.1 Hz, 1H), 7.28 (br. s., 1H), 6.79 (br. s., 1H), 6.69 (d, J=5.1 Hz, 1H), 4.54 (d, J=14.5 Hz, 2H), 2.79-3.10 (m, 2H), 2.39 (tt, J=11.4, 3.9 Hz, 1H), 1.77 (dd, J=12.8, 3.0 Hz, 2H), 1.26-1.58 (m, 2H); LCMS RT=4.017 min, m/z 241.0 [M+H⁺]; HRMS (ESI) m/z calcd for C₁₀H₁₄³⁵ClN₄O [M+H⁺] 241.0856.

### Example 13

### 1-(2-Chloropyrimidin-4-yl)Piperidine-4-Carboxamide (XJB02-012-2)

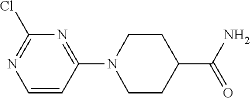 [SMILES: NC(=O)C1CCN(c2ccnc(Cl)n2)CC1]

¹H NMR (400 MHz, DMSO-d₆) δ ppm 8.04 (d, J=6.3 Hz, 1H), 7.32 (br. s., 1H), 6.84 (d, J=6.3 Hz, 1H), 6.81 (br. s., 1H), 4.29 (br. s., 2H), 2.91-3.04 (m, 2H), 2.42 (tt, J=11.3, 3.9 Hz, 1H), 1.79 (dd, J=13.1, 2.9 Hz, 2H), 1.40-1.54 (m, 2H); LCMS RT=4.017 min, m/z 241.0 [M+H⁺]; HRMS (ESI) m/z calcd for C₁₀H₁₄³⁵ClN₄O [M+H⁺] 241.0856.

### Example 14

### 1-(4-Chloro-5-Methylpyrimidin-2-yl)Piperidine-4-Carboxamide (XJB03-088-1)

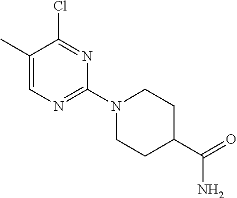 [SMILES: Cc1cnc(N2CCC(C(N)=O)CC2)nc1Cl]

A mixture of 2,4-dichloro-5-methylpyrimidine (1.02 g, 6.24 mmol), piperidine-4-carboxamide (800 mg, 11.7 mmol) in EtOH (8.00 mL) and TEA (4.00 mL) was heated in μW at 100° C. for 1 hour. The reaction mixture was cooled to room temperature, diluted with water and extracted with EtOAc and dichloromethane. The organic layer was separated, dried Na₂SO₄, and concentrated as light yellow solid. The crude mixture was purified with Biotage using 0-25% MeOH in CH₂Cl₂ to give 73.2 mg (5%) of XJB03-088-1 as a white solid and 1.04 g (65%) of XJB03-088-2 as a white solid. XJB03-088-1: ¹H NMR (400 MHz, DMSO-d₆) δ ppm 8.26 (s, 1H), 7.28 (br. s., 1H), 6.78 (br. s., 1H), 4.50 (d, J=13.1 Hz, 2H), 2.75-3.06 (m, 2H), 2.27-2.44 (m, 1H), 2.11 (s, 3H), 1.75 (dd, J=13.1, 2.3 Hz, 2H), 1.23-1.55 (m, 2H); LCMS RT=4.327 min, m/z 255.0 [M+H⁺]; HRMS (ESI) m/z calcd for C₁₁H₁₆³⁵ClN₄O [M+H⁺] 255.1013.

### Example 15

### 1-(2-Chloro-5-Methylpyrimidin-4-yl)Piperidine-4-Carboxamide (XJB03-088-2)

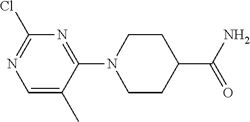 [SMILES: Cc1cnc(Cl)nc1N1CCC(C(N)=O)CC1]

¹H NMR (400 MHz, DMSO-d₆) δ ppm 8.00 (d, J=1.0 Hz, 1H), 7.30 (br. s., 1H), 6.80 (br. s., 1H), 3.82-4.29 (m, 2H), 2.78-3.05 (m, 2H), 2.39 (tt, J=11.4, 4.0 Hz, 1H), 2.18 (d, J=0.8 Hz, 3H), 1.78 (dd, J=13.1, 2.9 Hz, 2H), 1.42-1.69 (m, 2H); LCMS RT=3.368 min, m/z 255.0 [M+H⁺]; HRMS (ESI) m/z calcd for C₁₁H₁₆³⁵ClN₄O [M+H⁺] 255.1013.

### Example 16

### 1-(6-Chloropyrimidin-4-yl)Piperidine-4-Carboxamide (XJB03-091)

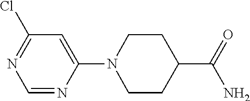 [SMILES: NC(=O)C1CCN(c2cc(Cl)ncn2)CC1]

A mixture of 4,6-dichloropyrimidine (930 mg, 6.24 mmol), piperidine-4-carboxamide (800 mg, 6.24 mmol) in ethanol (8.00 mL) and TEA (4.00 mL) was heated in μW at 100° C. for 1 hour. The reaction mixture was cooled to room temperature, diluted with water and extracted with EtOAc and dichloromethane. The organic layer was separated, dried Na₂SO₄, and concentrated as light yellow solid. The crude mixture was purified with Biotage with 0-25% MeOH in CH₂Cl₂ to give 1.06 g (71%) of product as a white solid: ¹H NMR (400 MHz, DMSO-d₆) δ ppm 8.31 (s, 1H), 7.28 (br. s., 1H), 6.96 (s, 1H), 6.80 (br. s., 1H), 4.34 (br. s., 2H), 2.86-3.06 (m, 2H), 2.41 (tt, 1H), 1.77 (dd, J=13.1, 2.9 Hz, 2H), 1.29-1.62 (m, 2H); LCMS RT=3.224 min, m/z 241.0 [M+H⁺]; HRMS (ESI) m/z calcd for C₁₀H₁₄³⁵ClN₄O [M+H⁺] 241.0856.

### Example 17

### 1-(4-Chloro-1,3,5-Triazin-2-yl)Piperidine-4-Carboxamide (XJB04-001)

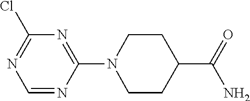 [SMILES: NC(=O)C1CCN(c2ncnc(Cl)n2)CC1]

A mixture of 2,4-dichloro-1,3,5-triazine (400 mg, 2.67 mmol), piperidine-4-carboxamide (342 mg, 2.67 mmol) in DMF (10.0 mL) was treated at 0° C. with DIPEA. The reaction mixture was stirred at 0° C. for 2 hour. The crude mixture was purified with Biotage with 0-20% MeOH in CH₂Cl₂ to give 324 mg (50%) of product as a white solid: ¹H NMR (400 MHz, DMSO-d₆) δ ppm 8.46 (s, 1H), 7.30 (br. s., 1H), 6.83 (br. s., 1H), 4.27-4.67 (m, 2H), 2.94-3.17 (m, 2H), 2.43 (tt, J=11.2, 4.0 Hz, 1H), 1.74-1.90 (m, 2H), 1.43-1.59 (m, 2H); LCMS RT=3.459 min, m/z 242.0 [M+H⁺]; HRMS (ESI) m/z calcd for C₉H₁₃³⁵ClN₅O [M+H⁺] 242.0809.

### Example 18

### 1-(4-(4-Cyanophenyl)Thiazol-2-yl)Piperidine-4-Carboxamide (XJB01-001)

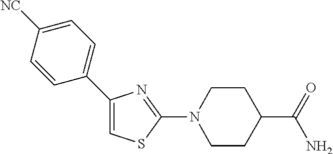 [SMILES: N#Cc1ccc(-c2csc(N3CCC(C(N)=O)CC3)n2)cc1]

The compound was prepared according to the general protocol A as a TFA salt: ¹H NMR (400 MHz, DMSO-d₆) δ 8.04-8.01 (m, 2H), 7.84-7.81 (m, 2H), 7.55 (s, 1H), 7.32 (br. s., 1H), 6.82 (br. s., 1H), 3.96 (dt, J=12.8, 3.2 Hz, 2H), 3.08 (td, J=12.4, 2.8 Hz, 2H), 2.35 (tt, J=11.6, 3.6 Hz, 1H), 1.81 (dd, J=13.2, 2.8 Hz, 2H), 1.61 (qd, J=12.3, 4.4 Hz, 2H); ¹⁹F NMR (376 MHz, DMSO-d₆) δ ppm −73.48 (s); LCMS RT=4.652 min, m/z 313.1 [M+H⁺]; HRMS (ESI) m/z calcd for C₁₆H₇N₄OS [M+H⁺] 313.1123.

### Example 19

### 1-(4-(4-Fluorophenyl)Thiazol-2-yl)Piperidine-4-Carboxamide (XJB01-002)

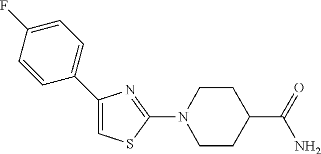 [SMILES: NC(=O)C1CCN(c2nc(-c3ccc(F)cc3)cs2)CC1]

The compound was prepared according to the general protocol A: ¹H NMR (400 MHz, DMSO-d₆) δ 7.91-7.87 (m, 2H), 7.32 (br. s., 1H), 7.23-7.18 (m, 3H), 6.82 (br. s., 1H), 3.96 (dt, J=12.8, 3.2 Hz, 2H), 3.06 (td, J=12.6, 2.8 Hz, 2H), 2.36 (tt, J=11.6, 3.8 Hz, 1H), 1.82 (dd, J=13.2, 2.8 Hz, 2H), 1.62 (qd, J=12.4, 4.4 Hz, 2H); ¹⁹F NMR (376 MHz, DMSO-d₆) δ ppm −114.65 (m); LCMS RT=4.390 min, m/z 306.1 [M+H⁺]; HRMS (ESI) m/z calcd for C₁₅H₁₇FN₃OS [M+H⁺] 306.1076.

### Example 20

### 1-(4-(4-Chlorophenyl)Thiazol-2-yl)Piperidine-4-Carboxamide (XJB01-003)

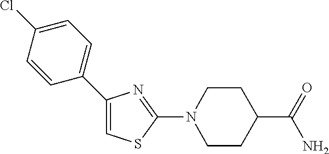 [SMILES: NC(=O)C1CCN(c2nc(-c3ccc(Cl)cc3)cs2)CC1]

The compound was prepared according to the general protocol A as a TFA salt: ¹H NMR (400 MHz, DMSO-d₆) δ 7.88-7.85 (m, 2H), 7.44-7.41 (m, 2H), 7.31 (br. s., 1H), 7.30 (s, 1H), 6.81 (br. s., 1H), 3.95 (dt, J=12.8, 3.2 Hz, 2H), 3.05 (td, J=12.5, 2.8 Hz, 2 H), 2.35 (tt, J=11.6, 3.6 Hz, 1H), 1.81 (dd, J=13.4, 2.6 Hz, 2H), 1.60 (qd, J=12.3, 4.2 Hz, 2H); ¹⁹F NMR (376 MHz, DMSO-d₆) δ ppm −73.68 (m); LCMS RT=4.954 min, m/z 322.1 [M+H⁺]; HRMS (ESI) m/z calcd for C₁₅H₁₇ClN₃OS [M+H⁺] 322.0781.

### Example 21

### 1-(4-(4-Methylphenyl)Thiazol-2-yl)Piperidine-4-Carboxamide (XJB01-004)

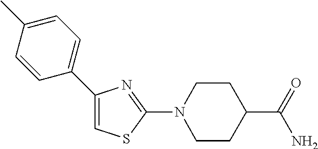 [SMILES: Cc1ccc(-c2csc(N3CCC(C(N)=O)CC3)n2)cc1]

The compound was prepared according to the general protocol A: ¹H NMR (400 MHz, DMSO-d₆) δ 7.75-7.72 (m, 2H), 7.32 (br. s., 1H), 7.19-7.17 (m, 2H), 7.16 (s, 1H), 6.82 (br. s., 1H), 3.96 (br. d., J=12.4 Hz, 2H), 3.05 (td, J=12.4, 2.8 Hz, 2H), 2.35 (tt, J=12.4, 3.8 Hz, 1H), 2.31 (s, 3H), 1.84-1.77 (m, 2H), 1.61 (qd, J=12.4, 3.8 Hz, 2H); LCMS RT=4.279 min, m/z 302.1 [M+H⁺]; HRMS (ESI) m/z calcd for C₁₆H₂₀N₃OS [M+H⁺] 302.1327.

### Example 22

### 1-(4-(4-Trifluoromethylphenyl)Thiazol-2-yl)Piperidine-4-Carboxamide (XJB01-005)

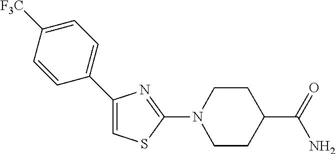 [SMILES: NC(=O)C1CCN(c2nc(-c3ccc(C(F)(F)F)cc3)cs2)CC1]

The compound was prepared according to the general protocol A as a TFA salt: ¹H NMR (400 MHz, DMSO-d₆) δ 8.08-8.05 (m, 2H), 7.74 (d, J=8.4 Hz, 2H), 7.49 (s, 1H), 7.33 (br. s., 1H), 6.83 (br. s., 1H), 3.98 (dt, J=12.4, 2.8 Hz, 2H), 3.09 (td, J=12.6, 2.8 Hz, 2H), 2.37 (tt, J=11.6, 3.8 Hz, 1H), 1.83 (dd, J=12.8, 2.8 Hz, 2H), 1.62 (qd, J=12.3, 4.0 Hz, 2H); ¹⁹F NMR (376 MHz, DMSO-d₆) δ ppm −60.84 (s), −73.66 (s); LCMS RT=5.476 min, m/z 356.1 [M+H⁺]; HRMS (ESI) m/z calcd for C₁₆H₁₇F₃N₃OS [M+H⁺] 356.1044.

### Example 23

### 1-(4-(4-Bromophenyl)Thiazol-2-yl)Piperidine-4-Carboxamide (XJB01-007)

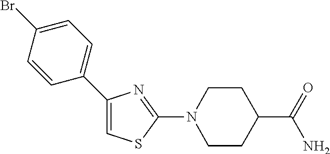 [SMILES: NC(=O)C1CCN(c2nc(-c3ccc(Br)cc3)cs2)CC1]

The compound was prepared according to the general protocol A: ¹H NMR (400 MHz, DMSO-d₆) δ 7.83-7.79 (m, 2H), 7.59-7.55 (m, 2H), 7.33 (s, 1H), 7.32 (br. s., 1H), 6.82 (br. s., 1H), 3.96 (br. d., J=12.8 Hz, 2H), 3.06 (td, J=12.6, 2.9 Hz, 2H), 2.36 (tt, J=11.6, 3.8 Hz, 1H), 1.82 (dd, J=13.0, 2.6 Hz, 2H), 1.61 (qd, J=12.4, 4.0 Hz, 2H); LCMS RT=5.165 min, m/z 366.0 [M+H⁺]; HRMS (ESI) m/z calcd for C₁₅H₁₇⁷⁹BrN₃OS [M+H⁺] 366.0276.

### Example 24

### 1-(4-(4-(Methylsulfonyl)Phenyl)Thiazol-2-yl)Piperidine-4-Carboxamide (XJB01-008)

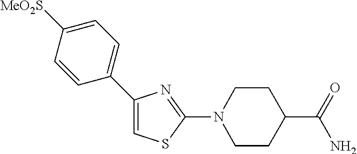 [SMILES: CS(=O)(=O)c1ccc(-c2csc(N3CCC(C(N)=O)CC3)n2)cc1]

The compound was prepared according to the general protocol A as a TFA salt: ¹H NMR (400 MHz, DMSO-d₆) δ 8.12-8.09 (m, 2H), 7.94-7.91 (m, 2H), 7.54 (s, 1H), 7.33 (br. s., 1H), 6.83 (br. s., 1H), 3.98 (br. d., J=12.8 Hz, 2H), 3.22 (s, 3H), 3.09 (td, J=12.5, 2.9 Hz, 2H), 2.37 (tt, J=11.4, 3.4 Hz, 1H), 1.83 (dd, J=13.2, 3.2 Hz, 2H), 1.62 (qd, J=12.3, 4.0 Hz, 2H); ¹⁹F NMR (376 MHz, DMSO-d₆) δ ppm −74.10 (s); LCMS RT=4.051 min, m/z 366.0 [M+H⁺]; HRMS (ESI) m/z calcd for C₁₆H₂₀N₃O₃S₂ [M+H⁺] 366.0946.

### Example 25

### 1-(4-(4-Methoxyphenyl)Thiazol-2-yl)Piperidine-4-Carboxamide (XJB01-009)

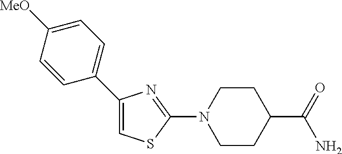 [SMILES: COc1ccc(-c2csc(N3CCC(C(N)=O)CC3)n2)cc1]

The compound was prepared according to the general protocol A as a TFA salt: ¹H NMR (400 MHz, DMSO-d₆) δ 7.79-7.76 (m, 2H), 7.32 (br. s., 1H), 7.07 (s, 1H), 6.95-6.92 (m, 2H), 6.82 (br. s., 1H), 3.95 (br. d., J=12.4 Hz, 2H), 3.77 (s, 3H), 3.04 (td, J=12.6, 2.8 Hz, 2H), 2.38-2.31 (m, 1H), 1.81 (dd, J=13.2, 2.8 Hz, 2H), 1.62 (qd, J=13.2, 3.8 Hz, 2H); ¹⁹F NMR (376 MHz, DMSO-d₆) δ ppm −73.48 (s); LCMS RT=3.968 min, m/z 318.1 [M+H⁺]; HRMS (ESI) m/z calcd for C₁₆H₂₀N₃O₂S [M+H⁺] 318.1276.

### Example 26

### 1-(4-(3-Bromophenyl)Thiazol-2-yl)Piperidine-4-Carboxamide (XJB01-011)

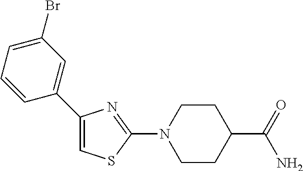 [SMILES: NC(=O)C1CCN(c2nc(-c3cccc(Br)c3)cs2)CC1]

The compound was prepared according to the general protocol A as a TFA salt: ¹H NMR (400 MHz, DMSO-d₆) δ 8.03 (t, J=1.8 Hz, 1H), 7.86 (ddd, J=8.0, 1.6, 0.8 Hz, 1H), 7.47 (dq, J=7.6, 0.9 Hz, 1H), 7.40 (s, 1H), 7.35 (t, J=7.8 Hz, 1H), 7.32 (br. s., 1H), 6.83 (br. s., 1H), 3.96 (dt, J=12.8, 3.0 Hz, 2H), 3.07 (td, J=12.4, 2.9 Hz, 2H), 2.36 (tt, J=11.6, 3.6 Hz, 1H), 1.82 (dd, J=13.6, 3.2 Hz, 2H), 1.62 (qd, J=12.4, 4.4 Hz, 2H); ¹⁹F NMR (376 MHz, DMSO-d₆) δ ppm −74.55 (s); LCMS RT=5.340 min, m/z 366.0 [M+H⁺]; HRMS (ESI) m/z calcd for C₁₅H₁₇⁷⁹BrN₃OS [M+H⁺] 366.0276.

### Example 27

### 1-(4-(2-Bromophenyl)Thiazol-2-yl)Piperidine-4-Carboxamide (XJB01-012)

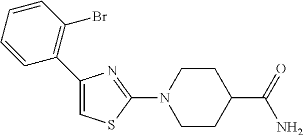 [SMILES: NC(=O)C1CCN(c2nc(-c3ccccc3Br)cs2)CC1]

The compound was prepared according to the general protocol A as a TFA salt: ¹H NMR (400 MHz, DMSO-d₆) δ 7.72 (dd, J=7.6, 1.6 Hz, 1H), 7.69 (dd, J=8.0, 1.2 Hz, 1H), 7.42 (dt, J=7.6, 1.2 Hz, 1H), 7.33 (br. s., 1H), 7.27 (ddd, J=8.0, 7.6, 1.6 Hz, 1H), 7.17 (s, 1H), 6.83 (br. s., 1H), 3.92 (dt, J=12.8, 3.2 Hz, 2H), 3.08 (td, J=12.4, 2.8 Hz, 2H), 2.36 (tt, J=11.4, 3.8 Hz, 1H), 1.82 (dd, J=13.2, 2.8 Hz, 2H), 1.62 (qd, J=12.3, 4.4 Hz, 2H); ¹⁹F NMR (376 MHz, DMSO-d₆) δ ppm −74.99 (s); LCMS RT=4.288 min, m/z 366.0 [M+H⁺]; HRMS (ESI) m/z calcd for C₁₅H₁₇⁷⁹BrN₃OS [M+H⁺] 366.0276.

### Example 28

### 1-(4-(Biphenyl-4-yl)Thiazol-2-yl)Piperidine-4-Carboxamide (XJB01-013)

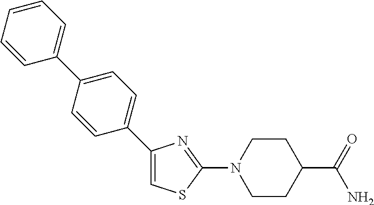 [SMILES: NC(=O)C1CCN(c2nc(-c3ccc(-c4ccccc4)cc3)cs2)CC1]

The compound was prepared according to the general protocol A as a TFA salt: ¹H NMR (400 MHz, DMSO-d₆) δ 7.96-7.94 (m, 2H), 7.72-7.68 (m, 4H), 7.49-7.46 (m, 2H), 7.39-7.35 (m, 1H), 7.33 (br. s., 1H), 7.31 (s, 1H), 6.83 (br. s., 1H), 4.00-3.97 (m, 2H), 3.11-3.05 (m, 2H), 2.40-2.32 (m, 1H), 1.86-1.78 (m, 2H), 1.68-1.58 (m, 2H); ¹⁹F NMR (376 MHz, DMSO-d₆) δ ppm −73.88 (s); LCMS RT=5.356 min, m/z 364.1 [M+H⁺]; HRMS (ESI) m/z calcd for C₂₁H₂₂N₃OS [M+H⁺] 364.4839.

### Example 29

### 1-(4-(4-Nitrophenyl)Thiazol-2-yl)Piperidine-4-Carboxamide (XJB01-014)

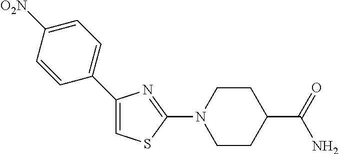 [SMILES: NC(=O)C1CCN(c2nc(-c3ccc([N+](=O)[O-])cc3)cs2)CC1]

The compound was prepared according to the general protocol A as a TFA salt: ¹H NMR (400 MHz, DMSO-d₆) δ 8.27-8.23 (m, 2H), 8.13-8.10 (m, 2H), 7.64 (s, 1H), 7.33 (br. s., 1H), 6.83 (br. s., 1H), 3.99 (br. d., J=12.8 Hz, 2H), 3.10 (td, J=12.4, 2.7 Hz, 2H), 2.40-2.32 (m, 1H), 1.83 (dd, J=13.2, 2.2 Hz, 2H), 1.62 (qd, J=12.4, 4.4 Hz, 2H); ¹⁹F NMR (376 MHz, DMSO-d₆) δ ppm −73.44 (s); LCMS RT=5.020 min, m/z 333.1 [M+H⁺]; HRMS (ESI) m/z calcd for C₁₅H₁₇N₄O₃S [M+H⁺] 333.1021.

### Example 30

### 1-(4-Phenylthiazol-2-yl)Piperidine-4-Carboxamide (XJB01-015)

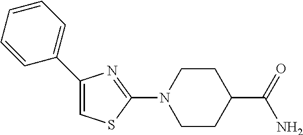 [SMILES: NC(=O)C1CCN(c2nc(-c3ccccc3)cs2)CC1]

The compound was prepared according to the general protocol A as a TFA salt: ¹H NMR (400 MHz, DMSO-d₆) δ 7.86-7.83 (m, 2H), 7.40-7.36 (m, 2H), 7.32 (br. s., 1H), 7.30-7.26 (m, 1H), 7.24 (s, 1H), 6.83 (br. s., 1H), 3.97 (dt, J=12.8, 3.2 Hz, 2H), 3.07 (td, J=12.4, 2.8 Hz, 2H), 2.36 (tt, J=11.4, 3.8 Hz, 1H), 1.82 (dd, J=13.6, 2.6 Hz, 2H), 1.62 (qd, J=12.3, 4.4 Hz, 2H); ¹⁹F NMR (376 MHz, DMSO-d₆) δ ppm −74.98 (s); LCMS RT=4.074 min, m/z 288.1 [M+H⁺]; HRMS (ESI) m/z calcd for C₁₅H₁₈N₃OS [M+H⁺] 288.1171.

### Example 31

### 4-(4-(4-Bromophenyl)Thiazol-2-yl)Morpholine (XJB01-016)

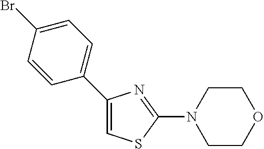 [SMILES: Brc1ccc(-c2csc(N3CCOCC3)n2)cc1]

Morpholine-4-carbothioamide (105 mg, 0.720 mmol) was added to a solution of 2-bromo-1-(4-bromophenyl)ethanone (100 mg, 0.360 mmol) in DMA (2.00 mL). The reaction mixture was stirred at room temperature for 0.5 h and poured into water. The precipitation was collected by filtration and dried overnight to afford a white solid: ¹H NMR (400 MHz, DMSO-d₆) δ 7.83-7.80 (m, 2H), 7.59-7.56 (m, 2H), 7.39 (s, 1H), 3.73 (t, J=5.0 Hz, 4H), 3.44 (dd, J=4.0, 5.2 Hz, 4H); LCMS RT=6.561 min, m/z 325.0 [M+H⁺]; HRMS (ESI) m/z calcd for C₁₃H₁₄⁷⁹BrN₂OS [M+H⁺] 325.0010.

### Example 32

### 4-(4-Bromophenyl)-2-(Piperidin-1-yl)Thiazole (XJB01-017)

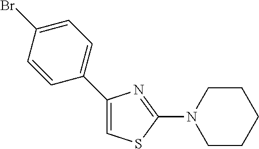 [SMILES: Brc1ccc(-c2csc(N3CCCCC3)n2)cc1]

Piperidine-1-carbothioamide (156 mg, 1.08 mmol) was added to a solution of 2-bromo-1-(4-bromophenyl)ethanone (250 mg, 0.899 mmol) in DMA (2.00 mL). The reaction mixture was stirred at room temperature for overnight and poured into water. The precipitation was collected by filtration and dried overnight to afford a white solid: ¹H NMR (400 MHz, DMSO-d₆) δ 7.82-7.78 (m, 2H), 7.58-7.55 (m, 2H), 7.30 (s, 1H), 3.47 (b, 4H), 2.51-2.49 (m, 6H); LCMS RT=6.992 min, m/z 323.0 [M+H⁺]; HRMS (ESI) m/z calcd for C₁₄H₁₆⁷⁹BrN₂S [M+H⁺] 323.0218.

### Example 33

### 1-(4-(3-Fluorophenyl)Thiazol-2-yl)Piperidine-4-Carboxamide (XJB01-019)

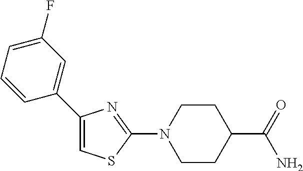 [SMILES: NC(=O)C1CCN(c2nc(-c3cccc(F)c3)cs2)CC1]

The compound was prepared according to the general protocol A as a TFA salt: ¹H NMR (400 MHz, DMSO-d₆) δ 7.70 (ddd, J=8.0, 1.6, 1.2 Hz, 1H), 7.64 (ddd, J=10.8, 2.4, 1.2 Hz, 1H), 7.42 (td, J=8.0, 6.4 Hz, 1H), 7.39 (s, 1H), 7.32 (br. s., 1H), 7.10 (tdd, J=8.4, 2.8, 0.8 Hz, 1H), 6.83 (br. s., 1H), 3.97 (dt, J=12.8, 3.2 Hz, 2H), 3.07 (td, J=12.6, 2.9 Hz, 2H), 2.36 (tt, J=11.4, 3.6 Hz, 1H), 1.82 (dd, J=13.2, 3.0 Hz, 2H), 1.62 (qd, J=12.4, 4.2 Hz, 2H); ¹⁹F NMR (376 MHz, DMSO-d₆) δ ppm −75.02 (s); LCMS RT=4.741 min, m/z 306.1 [M+H⁺]; HRMS (ESI) m/z calcd for C₁₅H₁₇FN₃OS [M+H⁺] 306.1076.

### Example 34

### 1-(4-(3-Chlorophenyl)Thiazol-2-yl)Piperidine-4-Carboxamide (XJB01-020)

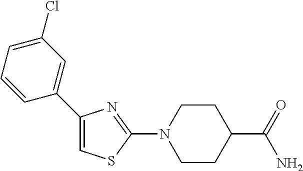 [SMILES: NC(=O)C1CCN(c2nc(-c3cccc(Cl)c3)cs2)CC1]

The compound was prepared according to the general protocol A as a TFA salt: ¹H NMR (400 MHz, DMSO-d₆) δ 7.89 (t, J=3.2 Hz, 1H), 7.82 (ddd, J=7.6, 1.6, 1.2 Hz, 1H), 7.41 (t, J=8.2 Hz, 1H), 7.40 (s, 1H), 7.33 (ddd, J=8.0, 2.0, 1.2 Hz, 1H), 7.32 (br. s., 1H), 6.83 (br. s., 1H), 3.97 (dt, J=12.8, 3.2 Hz, 2H), 3.07 (td, J=12.6, 2.9 Hz, 2H), 2.35 (tt, J=11.6, 3.6 Hz, 1H), 1.82 (dd, J=13.2, 2.6 Hz, 2H), 1.62 (qd, J=12.4, 4.4 Hz, 2H); ¹⁹F NMR (376 MHz, DMSO-d₆) δ ppm −74.23 (s); LCMS RT=5.155 min, m/z 322.0 [M+H⁺]; HRMS (ESI) m/z calcd for C₁₅H₁₇ClN₃OS [M+H⁺] 322.0781.

### Example 35

### 1-(4-(3-Methoxyphenyl)Thiazol-2-yl)Piperidine-4-Carboxamide (XJB01-022)

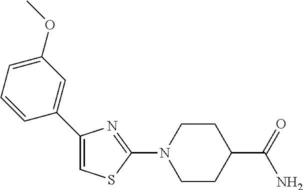 [SMILES: COc1cccc(-c2csc(N3CCC(C(N)=O)CC3)n2)c1]

The compound was prepared according to the general protocol A as a TFA salt: ¹H NMR (400 MHz, DMSO-d₆) δ ppm 7.37-7.46 (m, 2H), 7.34 (br. s., 1H), 7.25-7.31 (m, 2H), 6.86 (dd, J=8.2, 1.8 Hz, 1H), 6.83 (br. s., 1H), 3.91-4.01 (m, 2H), 3.79 (s, 3H), 3.06 (td, J=12.4, 2.5 Hz, 2H), 2.36 (tt, J=11.5, 3.4 Hz, 1H), 1.82 (dd, J=12.7, 2.5 Hz, 2H), 1.62 (qd, J=12.5, 3.9 Hz, 2H); ¹⁹F NMR (376 MHz, DMSO-d₆) δ ppm −74.97 (s); LCMS RT=4.260 min, m/z 318.1 [M+H⁺]; HRMS (ESI) m/z calcd for C₁₆H₂₀N₃O₂S [M+H⁺] 318.1276.

### Example 36

### 1-(4-(3-(Methylsulfonyl)Phenyl)Thiazol-2-yl)Piperidine-4-Carboxamide (XJB01-023)

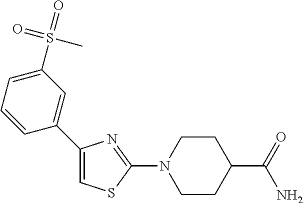 [SMILES: CS(=O)(=O)c1cccc(-c2csc(N3CCC(C(N)=O)CC3)n2)c1]

The compound was prepared according to the general protocol A as a TFA salt: ¹H NMR (400 MHz, DMSO-d₆) δ ppm 8.34 (t, J=1.6 Hz, 1H), 8.19 (ddd, J=8.0, 1.4, 1.2 Hz, 1H), 7.78-7.86 (m, J=7.5, 1.2, 0.9, 0.9 Hz, 1H), 7.67 (t, J=7.8 Hz, 1H), 7.49 (s, 1H), 7.33 (br. s., 1H), 6.83 (br. s., 1H), 3.88-4.06 (m, 2H), 3.25 (s, 3H), 3.09 (td, J=12.5, 3.1 Hz, 2H), 2.37 (tt, J=11.5, 3.8 Hz, 1H), 1.84 (dd, J=13.1, 2.9 Hz, 2H), 1.63 (qd, J=12.5, 4.1 Hz, 2H); ¹⁹F NMR (376 MHz, DMSO-d₆) δ ppm −74.53 (s); LCMS RT=4.094 min, m/z 366.0 [M+H⁺]; HRMS (ESI) m/z calcd for C₁₆H₂₀N₃O₃S₂ [M+H⁺] 366.0946.

### Example 37

### 1-(4-(3-Nitrophenyl)Thiazol-2-yl)Piperidine-4-Carboxamide (XJB01-024)

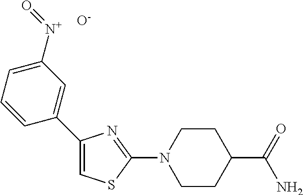

The compound was prepared according to the general protocol A as a TFA salt: ¹H NMR (400 MHz, DMSO-d₆) δ ppm 8.61-8.65 (m, 1H), 8.30 (dq, J=7.8, 0.9 Hz, 1H), 8.14 (ddd, J=8.2, 2.3, 1.2 Hz, 1H), 7.69 (t, J=8.0 Hz, 1H), 7.58 (s, 1H), 7.33 (br. s., 1H), 6.83 (br. s., 1H), 3.94-4.05 (m, 2H), 3.10 (td, J=12.6, 2.9 Hz, 2H), 2.37 (tt, J=11.4, 3.5 Hz, 1H), 1.84 (dd, J=13.5, 2.9 Hz, 2H), 1.63 (qd, J=12.5, 4.7 Hz, 2H); ¹⁹F NMR (376 MHz, DMSO-d₆) δ ppm −74.59 (s); LCMS RT=5.001 min, m/z 333.1 [M+H⁺]; HRMS (ESI) m/z calcd for C₁₅H₁₇N₄O₃S [M+H⁺] 333.1021.

### Example 38

### 1-(4-(2-Fluorophenyl)Thiazol-2-yl)Piperidine-4-Carboxamide (XJB01-025)

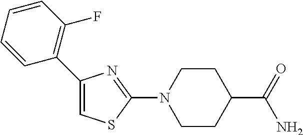 [SMILES: NC(=O)C1CCN(c2nc(-c3ccccc3F)cs2)CC1]

The compound was prepared according to the general protocol A as a TFA salt: ¹H NMR (400 MHz, DMSO-d₆) δ ppm 8.01-8.11 (m, 1H), 7.29-7.40 (m, 2H), 7.21-7.29 (m, 2H), 7.17 (d, J=2.7 Hz, 1H), 6.83 (br. s., 1H), 3.97 (dt, J=12.6, 3.3 Hz, 2H), 3.08 (td, J=12.5, 3.1 Hz, 2H), 2.29-2.42 (tt, J=11.4, 3.7 Hz, 1H), 1.82 (dd, J=13.5, 3.3 Hz, 2H), 1.62 (qd, J=12.4, 4.5 Hz, 2H); ¹⁹F NMR (376 MHz, DMSO-d₆) δ ppm −74.98 (s), −114.22-114.30 (m); LCMS RT=4.609 min, m/z 306.1 [M+H⁺]; HRMS (ESI) m/z calcd for C₁₅H₁₇FN₃OS [M+H⁺] 306.1076.

### Example 39

### 1-(4-(2-Chlorophenyl)Thiazol-2-yl)Piperidine-4-Carboxamide (XJB01-026)

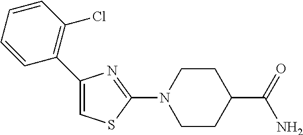 [SMILES: NC(=O)C1CCN(c2nc(-c3ccccc3Cl)cs2)CC1]

The compound was prepared according to the general protocol A as a TFA salt: ¹H NMR (400 MHz, DMSO-d₆) δ ppm 7.87 (dd, J=7.8, 2.0 Hz, 1H), 7.51 (dd, J=7.6, 1.4 Hz, 1H), 7.28-7.45 (m, 3H), 7.23-7.29 (m, 1H), 6.83 (br. s., 1H), 3.94 (dt, J=12.5, 3.1 Hz, 2H), 3.07 (td, J=12.5, 2.7 Hz, 2H), 2.36 (tt, J=11.5, 3.7 Hz, 1H), 1.82 (dd, J=13.5, 2.9 Hz, 2H), 1.62 (qd, J=12.4, 4.3 Hz, 2H); ¹⁹F NMR (376 MHz, DMSO-d₆) δ ppm −74.99 (s); LCMS RT=4.328 min, m/z 322.0 [M+H⁺]; HRMS (ESI) m/z calcd for C₁₅H₁₇ClN₃OS [M+H⁺] 322.0781.

### Example 40

### 1-(4-(2-(Trifluoromethyl)Phenyl)Thiazol-2-yl)Piperidine-4-Carboxamide (XJB01-027)

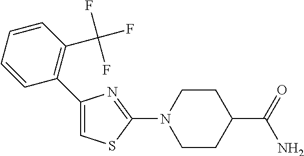 [SMILES: NC(=O)C1CCN(c2nc(-c3ccccc3C(F)(F)F)cs2)CC1]

The compound was prepared according to the general protocol A as a TFA salt: ¹H NMR (400 MHz, DMSO-d₆) δ ppm 7.80 (d, J=7.8 Hz, 1H), 7.62-7.74 (m, 2H), 7.58 (t, J=7.4 Hz, 1H), 7.32 (br. s., 1H), 6.90 (s, 1H), 6.83 (br. s., 1H), 3.90 (dt, J=12.5, 3.1 Hz, 2H), 3.06 (td, J=12.5, 3.1 Hz, 2H), 2.36 (tt, J=11.5, 3.7 Hz, 1H), 1.80 (dd, J=13.1, 2.9 Hz, 2H), 1.62 (qd, J=12.5, 4.3 Hz, 2H); ¹⁹F NMR (376 MHz, DMSO-d₆) δ ppm −56.43 (s), −75.03 (s); LCMS RT=4.485 min, m/z 356.1 [M+H⁺]; HRMS (ESI) m/z calcd for C₁₆H₁₇F₃N₃OS [M+H⁺] 356.1044.

### Example 41

### 1-(4-(2-Methoxyphenyl)Thiazol-2-yl)Piperidine-4-Carboxamide (XJB01-028)

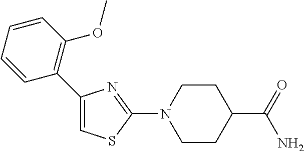 [SMILES: COc1ccccc1-c1csc(N2CCC(C(N)=O)CC2)n1]

The compound was prepared according to the general protocol A as a TFA salt: ¹H NMR (400 MHz, DMSO-d₆) δ ppm 8.05 (dd, J=7.6, 1.8 Hz, 1H), 7.33 (br. s., 1H), 7.31 (s, 1H), 7.25-7.31 (m, 1H), 7.09 (dd, J=8.2, 0.8 Hz, 1H), 7.00 (td, J=7.5, 1.0 Hz, 1H), 6.83 (br. s., 1H), 3.92-4.01 (m, 2H), 3.89 (s, 3H), 3.07 (td, J=12.5, 2.7 Hz, 2H), 2.36 (tt, J=11.5, 3.6 Hz, 1H), 1.82 (dd, J=13.1, 2.9 Hz, 2H), 1.63 (qd, J=11.7, 4.3 Hz, 2H); ¹⁹F NMR (376 MHz, DMSO-d₆) δ ppm −74.96 (s); LCMS RT=3.759 min, m/z 318.1 [M+H⁺]; HRMS (ESI) m/z calcd for C₁₆H₂₀N₃O₂S [M+H⁺] 318.1276.

### Example 42

### 1-(4-(2-(Methylsulfonyl)Phenyl)Thiazol-2-yl)Piperidine-4-Carboxamide (XJB01-029)

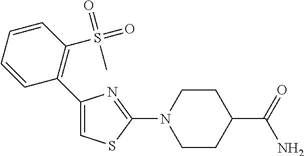 [SMILES: CS(=O)(=O)c1ccccc1-c1csc(N2CCC(C(N)=O)CC2)n1]

The compound was prepared according to the general protocol A as a TFA salt: ¹H NMR (400 MHz, DMSO-d₆) δ ppm 8.05 (dd, J=8.0, 1.4 Hz, 1H), 7.71-7.78 (m, 1H), 7.63-7.69 (m, 1H), 7.60 (dd, J=7.6, 1.4 Hz, 1H), 7.32 (br. s., 1H), 6.97 (s, 1H), 6.83 (br. s., 1H), 3.87 (dt, J=12.6, 3.5 Hz, 2H), 3.43 (s, 3H), 3.08 (td, J=12.4, 2.9 Hz, 2H), 2.36 (tt, J=11.3, 3.5 Hz, 1H), 1.81 (dd, J=13.5, 3.3 Hz, 2H), 1.62 (qd, J=11.7, 4.3 Hz, 2H); ¹⁹F NMR (376 MHz, DMSO-d₆) δ ppm −74.91 (s); LCMS RT=3.655 min, m/z 366.0 [M+H⁺]; HRMS (ESI) m/z calcd for C₁₆H₂₀N₃O₃S₂ [M+H⁺] 366.0946.

### Example 43

### 1-(4-(2-Nitrophenyl)Thiazol-2-yl)Piperidine-4-Carboxamide (XJB01-030)

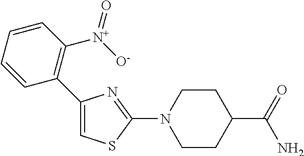 [SMILES: NC(=O)C1CCN(c2nc(-c3ccccc3[N+](=O)[O-])cs2)CC1]

The compound was prepared according to the general protocol A as a TFA salt: ¹H NMR (400 MHz, DMSO-d₆) δ ppm 7.78 (td, J=8.0, 1.2 Hz, 2H), 7.66 (td, J=7.6, 1.2 Hz, 1H), 7.49-7.58 (m, 1H), 7.30 (br. s., 1H), 7.19-7.25 (m, 1H), 6.82 (br. s., 1H), 3.80 (dt, J=12.8, 3.4 Hz, 2H), 3.01 (td, J=12.5, 3.1 Hz, 2H), 2.24-2.41 (m, 1H), 1.78 (dd, J=13.5, 3.3 Hz, 2H), 1.58 (qd, J=12.6, 4.3 Hz, 2H); ¹⁹F NMR (376 MHz, DMSO-d₆) δ ppm −74.90 (s); LCMS RT=4.354 min, m/z 333.1 [M+H⁺]; HRMS (ESI) m/z calcd for C₁₅H₁₇N₄O₃S [M+H⁺] 333.1021.

### Example 44

### Ethyl 1-(4-(4-Bromophenyl)Thiazol-2-yl)Piperidine-4-Carboxylate (XJB01-035)

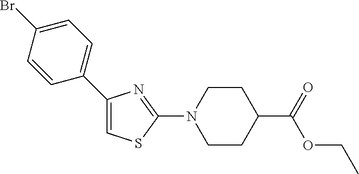 [SMILES: CCOC(=O)C1CCN(c2nc(-c3ccc(Br)cc3)cs2)CC1]

A mixture of ethyl 1-(4-bromothiazol-2-yl)piperidine-4-carboxylate (500 mg, 1.57 mmol), 4-bromophenylboronic acid (629 mg, 3.13 mmol) and tetrakis(triphenylphosphine)palladium (90.5 mg, 78.0 μmol) in CH₃CN (9.00 mL) and 2.0 M Na₂CO₃ aqueous solution (3.00 mL) was heated in μW at 100° C. for 30 min. The reaction mixture was cooled to room temperature, diluted with EtOAc, washed with H₂O. The organic layer was separated and dried (Na₂CO₃) and concentrated as brown oil which was purified with Biotage with 0-50% EtOAc in hexanes to give 297 mg (48%) product as a white solid: ¹H NMR (400 MHz, DMSO-d₆) δ ppm 7.78-7.84 (m, 2H), 7.52-7.61 (m, 2H), 7.34 (s, 1H), 4.09 (q, J=7.0 Hz, 2H), 3.90 (dt, J=12.9, 3.5 Hz, 2H), 3.15 (td, J=12.2, 2.9 Hz, 2H), 2.62 (tt, J=11.2, 4.1 Hz, 1H), 1.95 (dd, J=13.1, 3.3 Hz, 2H), 1.57-1.71 (m, 2H), 1.19 (t, J=7.2 Hz, 3H); LCMS RT=7.174 min, m/z 395.0 [M+H⁺]; HRMS (ESI) m/z calcd for C₁₇H₂₀⁷⁹BrN₂O₂S [M+H⁺] 395.0429.

### Example 45

### 1-(4-(4-Acetylphenyl)Thiazol-2-yl)Piperidine-4-Carboxamide (XJB01-036)

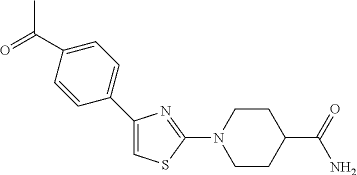 [SMILES: CC(=O)c1ccc(-c2csc(N3CCC(C(N)=O)CC3)n2)cc1]

The compound was prepared according to the general protocol A as a TFA salt: ¹H NMR (400 MHz, DMSO-d₆) δ ppm 7.91-8.04 (m, 4H), 7.49 (s, 1H), 7.33 (br. s., 1H), 6.83 (br. s., 1H), 3.98 (ddd, J=12.7, 3.3, 3.1 Hz, 2H), 3.08 (td, J=12.4, 2.9 Hz, 2H), 2.58 (s, 3H), 2.29-2.42 (m, 1H), 1.83 (dd, J=13.1, 3.3 Hz, 2H), 1.54-1.71 (m, 2H); ¹⁹F NMR (376 MHz, DMSO-d₆) δ ppm −74.84 (s); LCMS RT=4.320 min, m/z 330.1 [M+H⁺]; HRMS (ESI) m/z calcd for C₁₇H₂₀N₃O₂S [M+H⁺] 330.1276.

### Example 46

### 1-(4-M-Tolylthiazol-2-yl)Piperidine-4-Carboxamide (XJB01-037)

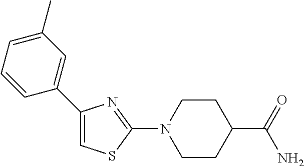 [SMILES: Cc1cccc(-c2csc(N3CCC(C(N)=O)CC3)n2)c1]

The compound was prepared according to the general protocol A as a TFA salt: ¹H NMR (400 MHz, DMSO-d₆) δ ppm 7.61-7.71 (m, 2H), 7.34 (br. s., 1H), 7.26 (t, J=7.6 Hz, 1H), 7.21 (s, 1H), 7.10 (d, J=7.4 Hz, 1H), 6.83 (br. s., 1H), 3.91-4.03 (m, 2H), 3.06 (td, J=12.5, 2.7 Hz, 2H), 2.34 (s, 3H), 2.31-2.41 (m, 1H), 1.82 (dd, J=12.7, 2.5 Hz, 2H), 1.62 (qd, J=12.5, 4.3 Hz, 2H); ¹⁹F NMR (376 MHz, DMSO-d₆) δ ppm −74.89 (s); LCMS RT=4.401 min, m/z 302.1 [M+H⁺]; HRMS (ESI) m/z calcd for C₁₆H₂₀N₃OS [M+H⁺] 302.1327.

### Example 47

### 1-(4-(Benzo[D][1,3]Dioxol-5-yl)Thiazol-2-yl)Piperidine-4-Carboxamide (XJB01-038)

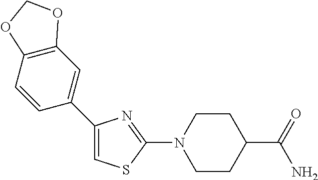 [SMILES: NC(=O)C1CCN(c2nc(-c3ccc4c(c3)OCO4)cs2)CC1]

The compound was prepared according to the general protocol A as a TFA salt: ¹H NMR (400 MHz, DMSO-d₆) δ ppm 7.36-7.43 (m, 2H), 7.31 (br. s., 1H), 7.11 (s, 1H), 6.88-6.95 (m, 1H), 6.82 (br. s., 1H), 6.03 (s, 2H), 3.86-4.04 (m, 2H), 3.04 (td, J=12.6, 2.9 Hz, 2H), 2.35 (tt, J=11.3, 3.1 Hz, 1H), 1.81 (dd, J=13.1, 2.9 Hz, 2H), 1.50-1.61 (m, J=12.3, 4.3 Hz, 2H); ¹⁹F NMR (376 MHz, DMSO-d₆) δ ppm −74.80 (s); LCMS RT=4.078 min, m/z 332.1 [M+H⁺]; HRMS (ESI) m/z calcd for C₁₆H₁₈N₃O₃S [M+H⁺] 332.1069.

### Example 48

### 1-(4-(2,3-Dihydrobenzo[B][1,4]Dioxin-6-yl)Thiazol-2-yl)Piperidine-4-Carboxamide (XJB01-039)

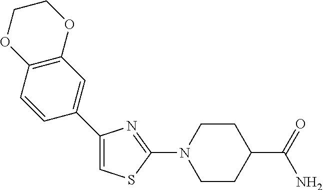 [SMILES: NC(=O)C1CCN(c2nc(-c3ccc4c(c3)OCCO4)cs2)CC1]

The compound was prepared according to the general protocol A as a TFA salt: ¹H NMR (400 MHz, DMSO-d₆) δ ppm 7.26-7.44 (m, 3H), 7.08 (s, 1H), 6.85 (d, J=8.2 Hz, 1H), 6.83 (br. s., 1H), 4.25 (s, 4H), 3.88-3.99 (m, 2H), 3.07 (td, J=12.5, 2.7 Hz, 2H), 2.36 (tt, J=11.4, 3.5 Hz, 1H), 1.82 (dd, J=13.1, 2.9 Hz, 2H), 1.62 (qd, J=12.4, 4.3 Hz, 2H); ¹⁹F NMR (376 MHz, DMSO-d₆) δ ppm −75.15 (s); LCMS RT=4.030 min, m/z 346.1 [M+H⁺]; HRMS (ESI) m/z calcd for C₁₇H₂₀N₃O₃S [M+H⁺] 346.1225.

### Example 49

### 1-(4-(Naphthalen-1-yl)Thiazol-2-yl)Piperidine-4-Carboxamide (XJB01-040)

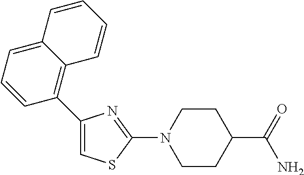 [SMILES: NC(=O)C1CCN(c2nc(-c3cccc4ccccc34)cs2)CC1]

The compound was prepared according to the general protocol A as a TFA salt: ¹H NMR (400 MHz, DMSO-d₆) δ ppm 8.33-8.44 (m, 1H), 7.88-8.02 (m, 2H), 7.62-7.70 (m, 1H), 7.48-7.57 (m, 3H), 7.33 (br. s., 1H), 7.04 (s, 1H), 6.83 (br. s., 1H), 3.90-4.02 (m, 2H), 3.11 (td, J=12.5, 2.7 Hz, 2H), 2.29-2.43 (m, 1H), 1.83 (dd, J=13.1, 2.5 Hz, 2H), 1.58-1.73 (m, 2H); ¹⁹F NMR (376 MHz, DMSO-d₆) δ ppm −74.65 (s); LCMS RT=4.327 min, m/z 338.1 [M+H⁺]; HRMS (ESI) m/z calcd for C₁₉H₂₀N₃OS [M+H⁺] 338.1327.

### Example 50

### 1-(4-(4-Oxo-4H-Chromen-6-yl)Thiazol-2-yl)Piperidine-4-Carboxamide (XJB01-041)

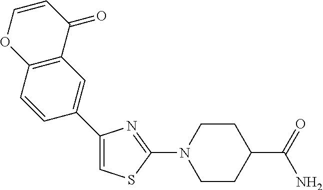 [SMILES: NC(=O)C1CCN(c2nc(-c3ccc4occc(=O)c4c3)cs2)CC1]

The compound was prepared according to the general protocol A as a TFA salt: ¹H NMR (400 MHz, DMSO-d₆) δ ppm 8.47 (d, J=2.3 Hz, 1H), 8.20-8.34 (m, 2H), 7.67 (d, J=8.6 Hz, 1H), 7.45 (s, 1H), 7.33 (br. s., 1H), 6.83 (br. s., 1H), 6.37 (d, J=6.3 Hz, 1H), 3.99 (dt, J=12.5, 3.1 Hz, 2H), 3.09 (td, J=12.6, 2.9 Hz, 2H), 2.31-2.43 (m, 1H), 1.84 (dd, J=13.3, 2.7 Hz, 2H), 1.54-1.74 (m, 2H); ¹⁹F NMR (376 MHz, DMSO-d₆) δ ppm −74.64 (s); LCMS RT=4.093 min, m/z 356.1 [M+H⁺]; HRMS (ESI) m/z calcd for C₁₈H₁₈N₃O₃S [M+H⁺] 356.1069.

### Example 51

### 1-(4-(Thiophen-2-yl)Thiazol-2-yl)Piperidine-4-Carboxamide (XJB01-042)

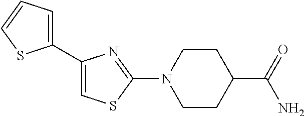 [SMILES: NC(=O)C1CCN(c2nc(-c3cccs3)cs2)CC1]

The compound was prepared according to the general protocol A as a TFA salt: ¹H NMR (400 MHz, DMSO-d₆) δ ppm 7.43 (dd, J=4.3, 2.3 Hz, 2H), 7.28-7.35 (m, 1H), 7.01-7.10 (m, 2H), 6.83 (br. s., 1H), 3.91 (ddd, J=12.7, 3.1, 2.9 Hz, 2H), 3.04 (td, J=12.5, 2.7 Hz, 2H), 2.35 (tt, J=11.5, 3.7 Hz, 1H), 1.81 (dd, J=13.3, 3.1 Hz, 2H), 1.60 (qd, J=12.4, 4.3 Hz, 2H); ¹⁹F NMR (376 MHz, DMSO-d₆) δ ppm −74.92 (s); LCMS RT=4.474 min, m/z 294.0 [M+H⁺]; HRMS (ESI) m/z calcd for C₁₃H₁₆N₃OS₂ [M+H⁺] 294.0735.

### Example 52

### 1-(4-(3,5-dimethylisoxazol-4-yl)thiazol-2-yl)piperidine-4-carboxamide (XJB01-044)

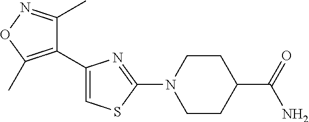 [SMILES: Cc1noc(C)c1-c1csc(N2CCC(C(N)=O)CC2)n1]

The compound was prepared according to the general protocol A as a TFA salt: ¹H NMR (400 MHz, DMSO-d₆) δ ppm 7.32 (br. s., 1H), 6.88 (s, 1H), 6.83 (br. s., 1H), 3.91 (dt, J=12.4, 3.2 Hz, 2H), 3.06 (td, J=12.5, 3.1 Hz, 2H), 2.57 (s, 3H), 2.35 (s, 3H), 2.29-2.43 (m, 1H), 1.81 (dd, J=13.5, 2.9 Hz, 2H), 1.61 (qd, J=12.5, 4.3 Hz, 2H); ¹⁹F NMR (376 MHz, DMSO-d₆) δ ppm −74.99 (s); LCMS RT=3.771 min, m/z 307.1 [M+H⁺]; HRMS (ESI) m/z calcd for C₁₄H₁₉N₄O₂S [M+H⁺] 307.1229.

### Example 53

### 1-(4-(2-Aminophenyl)Thiazol-2-yl)Piperidine-4-Carboxamide (XJB01-045)

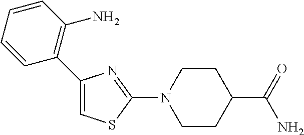 [SMILES: NC(=O)C1CCN(c2nc(-c3ccccc3N)cs2)CC1]

The compound was prepared according to the general protocol A as a TFA salt: ¹H NMR (400 MHz, DMSO-d₆) δ ppm 7.65 (dd, J=7.8, 1.6 Hz, 1H), 7.34 (br. s., 1H), 7.16-7.28 (m, 2H), 6.98-7.13 (m, 2H), 6.84 (br. s., 1H), 3.88-4.07 (m, 2H), 3.10 (td, J=12.5, 3.1 Hz, 2H), 2.38 (tt, J=11.5, 3.6 Hz, 1H), 1.83 (dd, J=13.1, 2.9 Hz, 2H), 1.64 (qd, J=12.4, 4.5 Hz, 2H); ¹⁹F NMR (376 MHz, DMSO-d₆) δ ppm −74.68 (s); LCMS RT=3.445 min, m/z 303.1 [M+H⁺]; HRMS (ESI) m/z calcd for C₁₅H₁₉N₄OS [M+H⁺] 303.1280.

### Example 54

### 1-(4-(Pyridin-3-yl)Thiazol-2-yl)Piperidine-4-Carboxamide (XJB01-046)

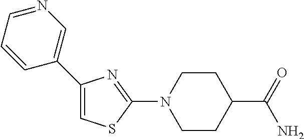 [SMILES: NC(=O)C1CCN(c2nc(-c3cccnc3)cs2)CC1]

The compound was prepared according to the general protocol A as a TFA salt: ¹H NMR (400 MHz, DMSO-d₆) δ ppm 9.14 (dd, J=2.0, 0.8 Hz, 1H), 8.60 (dd, J=5.1, 1.6 Hz, 1H), 8.47 (dt, J=8.2, 2.0 Hz, 1H), 7.62-7.72 (m, 1H), 7.56 (s, 1H), 7.33 (br. s., 1H), 6.83 (br. s., 1H), 3.92-4.04 (m, 2H), 3.10 (td, J=12.7, 3.1 Hz, 2H), 2.28-2.43 (m, 1H), 1.83 (dd, J=13.5, 2.9 Hz, 2H), 1.56-1.70 (m, 2H); ¹⁹F NMR (376 MHz, DMSO-d₆) δ ppm −74.45 (s); LCMS RT=2.988 min, m/z 289.0 [M+H⁺]; HRMS (ESI) m/z calcd for C₁₄H₁₇N₄OS [M+H⁺] 289.1123.

### Example 55

### 1-(4-(3-Acetylphenyl)Thiazol-2-yl)Piperidine-4-Carboxamide (XJB01-047)

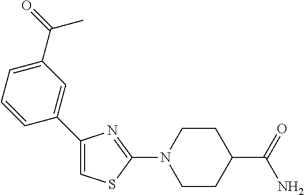 [SMILES: CC(=O)c1cccc(-c2csc(N3CCC(C(N)=O)CC3)n2)c1]

The compound was prepared according to the general protocol A as a TFA salt: ¹H NMR (400 MHz, DMSO-d₆) δ ppm 8.35 (d, J=2.0 Hz, 1H), 8.08 (dd, J=7.8, 1.2 Hz, 1H), 7.85 (dd, J=7.8, 1.2 Hz, 1H), 7.51 (t, J=7.6 Hz, 1H), 7.39 (s, 1H), 7.31 (br. s., 1H), 6.80 (br. s., 1H), 3.88-4.03 (m, 2H), 3.05 (td, J=12.5, 2.7 Hz, 2H), 2.59 (s, 3H), 2.29-2.38 (m, 1H), 1.76-1.84 (m, 2H), 1.60 (qd, J=12.4, 4.5 Hz, 2H); ¹⁹F NMR (376 MHz, DMSO-d₆) δ ppm −76.01 (s); LCMS RT=4.223 min, m/z 330.1 [M+H⁺]; HRMS (ESI) m/z calcd for C₁₇H₂₀N₃O₂S [M+H⁺] 330.1276.

### Example 56

### 1-(4-(Naphthalen-2-yl)Thiazol-2-yl)Piperidine-4-Carboxamide (XJB01-048)

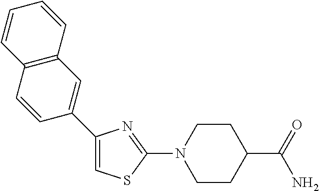 [SMILES: NC(=O)C1CCN(c2nc(-c3ccc4ccccc4c3)cs2)CC1]

The compound was prepared according to the general protocol A: ¹H NMR (400 MHz, DMSO-d₆) δ ppm 8.40 (s, 1H), 7.83-8.04 (m, 4H), 7.44-7.55 (m, 2H), 7.39 (s, 1H), 7.32 (br. s., 1H), 6.81 (br. s., 1H), 4.03 (dd, 2H), 3.10 (td, J=12.5, 3.1 Hz, 2H), 2.38 (tt, J=11.5, 3.6 Hz, 1H), 1.86 (dd, J=13.1, 2.9 Hz, 2H), 1.58-1.73 (m, 2H); LCMS RT=5.008 min, m/z 338.1 [M+H⁺]; HRMS (ESI) m/z calcd for C₁₉H₂₀N₃OS [M+H⁺] 338.1327.

### Example 57

### 1-(4-(3-Aminophenyl)Thiazol-2-yl)Piperidine-4-Carboxamide (XJB01-049)

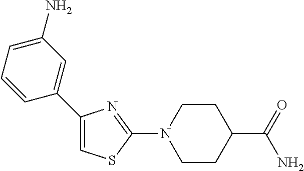 [SMILES: NC(=O)C1CCN(c2nc(-c3cccc(N)c3)cs2)CC1]

The compound was prepared according to the general protocol A as a TFA salt: ¹H NMR (400 MHz, DMSO-d₆) δ ppm 7.57-7.72 (m, 2H), 7.28-7.44 (m, 2H), 7.25 (s, 1H), 7.04 (t, J=7.8 Hz, 1H), 6.81 (br. s., 1H), 3.83-4.06 (m, 4H), 3.05 (td, J=12.7, 3.1 Hz, 2H), 2.26-2.39 (m, 1H), 1.79 (dd, J=13.9, 2.9 Hz, 2H), 1.59 (qd, J=12.5, 3.7 Hz, 2H); ¹⁹F NMR (376 MHz, DMSO-d₆) δ ppm −74.57 (s); LCMS RT=3.055 min, m/z 303.1 [M+H⁺]; HRMS (ESI) m/z calcd for C₁₅H₁₉N₄OS [M+H⁺] 303.1280.

### Example 58

### 1-(4-(3-Cyanophenyl)Thiazol-2-yl)Piperidine-4-Carboxamide (XJB01-051)

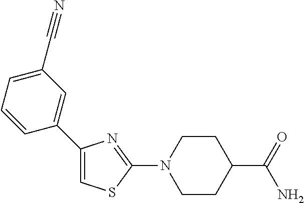 [SMILES: N#Cc1cccc(-c2csc(N3CCC(C(N)=O)CC3)n2)c1]

The compound was prepared according to the general protocol A as a TFA salt: ¹H NMR (400 MHz, DMSO-d₆) δ ppm 8.25 (t, J=1.4 Hz, 1H), 8.12-8.18 (m, 1H), 7.67-7.74 (m, 1H), 7.57 (t, J=7.8 Hz, 1H), 7.46 (s, 1H), 7.30 (br. s., 1H), 6.80 (br. s., 1H), 3.90-4.01 (m, 2H), 3.05 (td, J=12.5, 2.7 Hz, 2H), 2.33 (tt, J=11.4, 3.7 Hz, 1H), 1.80 (dd, J=13.5, 2.9 Hz, 2H), 1.59 (qd, J=12.1, 4.3 Hz, 2H); ¹⁹F NMR (376 MHz, DMSO-d₆) δ ppm −74.58 (s); LCMS RT=4.665 min, m/z 313.1 [M+H⁺]; HRMS (ESI) m/z calcd for C₁₆H₁₇N₄OS [M+H⁺] 313.1123.

### Example 59

### 1-(4-(2-(Dimethylamino)Phenyl)Thiazol-2-yl)Piperidine-4-Carboxamide (XJB01-052)

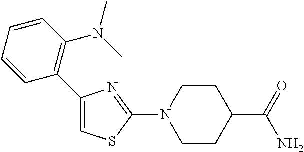 [SMILES: CN(C)c1ccccc1-c1csc(N2CCC(C(N)=O)CC2)n1]

The compound was prepared according to the general protocol A as a TFA salt: ¹H NMR (400 MHz, DMSO-d₆) δ ppm 7.84-8.11 (m, 2H), 7.67 (br. s., 1H), 7.56 (br. s., 2H), 7.36 (br. s., 1H), 6.86 (br. s., 1H), 3.80-3.98 (m, 2H), 3.06-3.49 (m, 8H), 2.36-2.48 (m, 1H), 1.87 (dd, J=13.5, 2.9 Hz, 2H), 1.57-1.76 (m, 2H); ¹⁹F NMR (376 MHz, DMSO-d₆) δ ppm −74.24 (s); LCMS RT=3.599 min, m/z 331.1 [M+H⁺]; HRMS (ESI) m/z calcd for C₁₇H₂₃N₄OS [M+H⁺] 331.1593.

### Example 60

### 1-(4-(6-(Piperidin-1-yl)Pyridin-3-yl)Thiazol-2-yl)Piperidine-4-Carboxamide (XJB01-053)

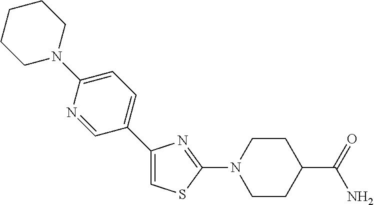 [SMILES: NC(=O)C1CCN(c2nc(-c3ccc(N4CCCCC4)nc3)cs2)CC1]

The compound was prepared according to the general protocol A as a TFA salt: ¹H NMR (400 MHz, DMSO-d₆) δ ppm 8.22-8.40 (m, 2H), 7.30-7.46 (m, 3H), 6.83 (br. s., 1H), 3.88-4.05 (m, 2H), 3.54-3.76 (m, 4H), 3.08 (td, J=12.5, 2.7 Hz, 2H), 2.37 (tt, J=11.4, 3.7 Hz, 1H), 1.82 (dd, J=13.1, 2.9 Hz, 2H), 1.51-1.73 (m, 8H); ¹⁹F NMR (376 MHz, DMSO-d₆) δ ppm −74.74 (s); LCMS RT=3.702 min, m/z 372.1 [M+H⁺]; HRMS (ESI) m/z calcd for C₁₉H₂₆N₅OS [M+H⁺] 372.1858.

### Example 61

### 1-(4-(4-Acetamidophenyl)Thiazol-2-yl)Piperidine-4-Carboxamide (XJB01-054)

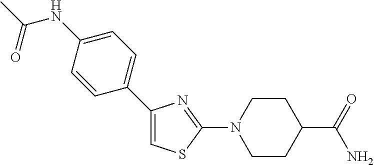 [SMILES: CC(=O)Nc1ccc(-c2csc(N3CCC(C(N)=O)CC3)n2)cc1]

The compound was prepared according to the general protocol A as a TFA salt: ¹H NMR (400 MHz, DMSO-d₆) δ ppm 9.98 (s, 1H), 7.72-7.79 (m, 2H), 7.58 (d, J=8.6 Hz, 2H), 7.32 (br. s., 1H), 7.11 (s, 1H), 6.82 (br. s., 1H), 3.96 (ddd, J=12.8, 3.9, 3.6 Hz, 2H), 3.05 (td, J=12.5, 2.7 Hz, 2H), 2.29-2.41 (m, 1H), 2.04 (s, 3H), 1.82 (dd, J=13.3, 2.3 Hz, 2H), 1.53-1.70 (m, 2H); ¹⁹F NMR (376 MHz, DMSO-d₆) δ ppm −74.76 (s); LCMS RT=3.405 min, m/z 345.1 [M+H⁺]; HRMS (ESI) m/z calcd for C₁₇H₂₁N₄O₂S [M+H⁺] 345.1385.

### Example 62

### 1-(4-(3-(Dimethylcarbamoyl)Phenyl)Thiazol-2-yl)Piperidine-4-Carboxamide (XJB01-056)

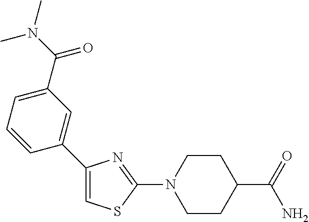 [SMILES: CN(C)C(=O)c1cccc(-c2csc(N3CCC(C(N)=O)CC3)n2)c1]

The compound was prepared according to the general protocol A as a TFA salt: ¹H NMR (400 MHz, DMSO-d₆) δ ppm 7.91 (ddd, J=8.0, 1.4, 1.2 Hz, 1H), 7.85 (t, J=1.4 Hz, 1H), 7.44 (t, J=7.6 Hz, 1H), 7.35 (s, 1H), 7.32 (br. s., 1H), 7.28 (dt, J=7.7, 1.4 Hz, 1H), 6.82 (br. s., 1H), 3.93-4.02 (m, 2H), 3.07 (td, J=12.5, 2.7 Hz, 2H), 3.00 (br. s., 3H), 2.92 (br. s., 3H), 2.36 (tt, J=11.4, 3.3 Hz, 1H), 1.82 (dd, J=13.3, 2.7 Hz, 2H), 1.62 (qd, J=12.4, 4.3 Hz, 2H); ¹⁹F NMR (376 MHz, DMSO-d₆) δ ppm −75.01 (s); LCMS RT=3.697 min, m/z 359.1 [M+H⁺]; HRMS (ESI) m/z calcd for C₁₈H₂₃N₄O₂S [M+H⁺] 359.1542.

### Example 63

### 1-(4-(3-(Dimethylamino)Phenyl)Thiazol-2-yl)Piperidine-4-Carboxamide (XJB01-057)

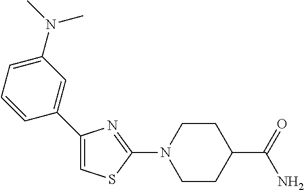 [SMILES: CN(C)c1cccc(-c2csc(N3CCC(C(N)=O)CC3)n2)c1]

The compound was prepared according to the general protocol A as a TFA salt: ¹H NMR (400 MHz, DMSO-d₆) δ ppm 7.59 (br. s., 1H), 7.40-7.52 (m, 1H), 7.30-7.41 (m, 2H), 7.25 (s, 1H), 7.06 (d, J=7.4 Hz, 1H), 6.83 (br. s., 1H), 3.93-4.04 (m, 2H), 3.00-3.14 (m, 8H), 2.37 (tt, J=11.5, 3.6 Hz, 1H), 1.83 (dd, J=13.3, 2.7 Hz, 2H), 1.56-1.71 (m, 2H); ¹⁹F NMR (376 MHz, DMSO-d₆) δ ppm −74.97 (s); LCMS RT=3.372 min, m/z 331.1 [M+H⁺]; HRMS (ESI) m/z calcd for C₁₇H₂₃N₄OS [M+H⁺] 331.1593.

### Example 64

### 1-(4-(Dibenzo[b,d]Thiophen-1-yl)Thiazol-2-yl)Piperidine-4-Carboxamide (XJB01-058)

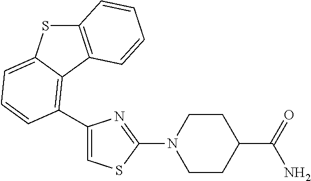 [SMILES: NC(=O)C1CCN(c2nc(-c3cccc4sc5ccccc5c34)cs2)CC1]

The compound was prepared according to the general protocol A: ¹H NMR (400 MHz, DMSO-d₆) δ ppm 8.24-8.40 (m, 2H), 7.92-8.06 (m, 2H), 7.52 (t, J=7.6 Hz, 1H), 7.43-7.50 (m, 3H), 7.32 (br. s., 1H), 6.82 (br. s., 1H), 4.06 (dt, J=12.6, 3.3 Hz, 2H), 3.16 (td, J=12.5, 3.1 Hz, 2H), 2.39 (tt, J=11.4, 3.7 Hz, 1H), 1.86 (dd, J=12.9, 2.7 Hz, 2H), 1.60-1.74 (m, 2H); LCMS RT=6.166 min, m/z 394.1 [M+H⁺]; HRMS (ESI) m/z calcd for C₂₁H₂₀N₃OS₂ [M+H⁺] 394.1048.

### Example 65

### 1-(4-(1-Methyl-1H-Indol-5-yl)Thiazol-2-yl)Piperidine-4-Carboxamide (XJB01-059)

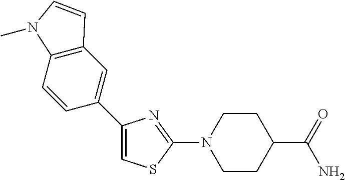 [SMILES: Cn1ccc2cc(-c3csc(N4CCC(C(N)=O)CC4)n3)ccc21]

The compound was prepared according to the general protocol A as a TFA salt: ¹H NMR (400 MHz, DMSO-d₆) δ ppm 8.06 (d, J=1.2 Hz, 1H), 7.65 (dd, J=8.6, 1.6 Hz, 1H), 7.42 (d, J=8.6 Hz, 1H), 7.23-7.37 (m, 2H), 7.08 (s, 1H), 6.83 (br. s., 1H), 6.45 (dd, J=3.1, 0.8 Hz, 1H), 3.91-4.08 (m, 2H), 3.79 (s, 3H), 3.09 (td, J=12.5, 3.1 Hz, 2H), 2.37 (tt, J=11.5, 3.7 Hz, 1H), 1.84 (dd, J=13.1, 2.9 Hz, 2H), 1.54-1.73 (m, 2H); ¹⁹F NMR (376 MHz, DMSO-d₆) δ ppm −74.94 (s); LCMS RT=4.045 min, m/z 341.1 [M+H⁺]; HRMS (ESI) m/z calcd for C₁₈H₂₁N₄OS [M+H⁺] 341.1436.

### Example 66

### 1-(4-(Biphenyl-2-yl)Thiazol-2-yl)Piperidine-4-Carboxamide (XJB01-060)

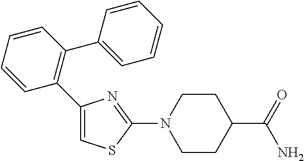 [SMILES: NC(=O)C1CCN(c2nc(-c3ccccc3-c3ccccc3)cs2)CC1]

The compound was prepared according to the general protocol A as a TFA salt: ¹H NMR (400 MHz, DMSO-d₆) δ ppm 7.65-7.85 (m, 1H), 7.25-7.49 (m, 7H), 7.14-7.25 (m, 2H), 6.81 (br. s., 1H), 6.12 (s, 1H), 3.70-3.83 (m, 2H), 2.95 (td, J=12.4, 2.5 Hz, 2H), 2.29 (tt, J=11.2, 3.9 Hz, 1H), 1.73 (dd, J=13.3, 3.1 Hz, 2H), 1.44-1.60 (m, 2H); ¹⁹F NMR (376 MHz, DMSO-d₆) δ ppm −74.85 (s); LCMS RT=4.462 min, m/z 364.1 [M+H⁺]; HRMS (ESI) m/z calcd for C₂₁H₂₂N₃OS [M+H⁺] 364.1484.

### Example 67

### 1-(4-(2-Cyanophenyl)Thiazol-2-yl)Piperidine-4-Carb Oxamide (XJB01-061)

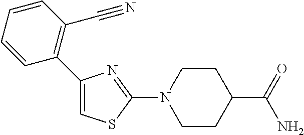 [SMILES: N#Cc1ccccc1-c1csc(N2CCC(C(N)=O)CC2)n1]

The compound was prepared according to the general protocol A as a TFA salt: ¹H NMR (400 MHz, DMSO-d₆) δ ppm 7.97 (dd, J=8.4, 1.0 Hz, 1H), 7.85 (dd, J=7.8, 1.6 Hz, 1H), 7.65-7.78 (m, 1H), 7.45-7.54 (m, 1H), 7.43 (s, 1H), 7.32 (br. s., 1H), 6.82 (br. s., 1H), 3.98 (dt, J=12.4, 3.2 Hz, 2H), 3.10 (td, J=12.5, 3.1 Hz, 2H), 2.28-2.42 (m, 1H), 1.82 (dd, J=13.5, 3.3 Hz, 2H), 1.56-1.71 (m, 2H); ¹⁹F NMR (376 MHz, DMSO-d₆) δ ppm −74.86 (s); LCMS RT=4.527 min, m/z 313.1 [M+H⁺]; HRMS (ESI) m/z calcd for C₁₆H₁₇N₄OS [M+H⁺] 313.1123.

### Example 68

### 1-(4-(Benzofuran-2-yl)Thiazol-2-yl)Piperidine-4-Carboxamide (XJB01-063)

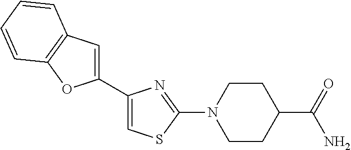 [SMILES: NC(=O)C1CCN(c2nc(-c3cc4ccccc4o3)cs2)CC1]

The compound was prepared according to the general protocol A as a TFA salt: ¹H NMR (400 MHz, DMSO-d₆) δ ppm 7.46-7.74 (m, 3H), 7.18-7.40 (m, 3H), 7.09 (d, J=0.8 Hz, 1H), 6.83 (br. s., 1H), 3.97 (dt, J=12.8, 3.2 Hz, 2H), 3.10 (td, J=12.7, 3.1 Hz, 2H), 2.26-2.42 (m, 1H), 1.74-1.91 (m, 2H), 1.64 (qd, J=12.7, 4.5 Hz, 2H); ¹⁹F NMR (376 MHz, DMSO-d₆) δ ppm −74.78 (s); LCMS RT=5.337 min, m/z 328.1 [M+H⁺]; HRMS (ESI) m/z calcd for C₁₇H₁₈N₃O₂S [M+H⁺] 328.1120.

### Example 69

### 1-(4-(4-Bromophenyl)Thiazol-2-yl)Piperidine-4-Carboxylic Acid (XJB01-064)

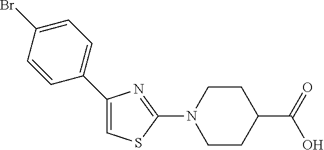 [SMILES: O=C(O)C1CCN(c2nc(-c3ccc(Br)cc3)cs2)CC1]

LiOH (1.01 g, 42.2 mmol) was added to a solution of ethyl 1-(4-(4-bromophenyl)thiazol-2-yl)piperidine-4-carboxylate (3.34 g, 8.45 mmol) in THF (24.0 mL) and H₂O (8.00 mL) at room temperature. The reaction mixture was stirred at room temperature for 24 hours, diluted with 100 mL CH₂Cl₂, and washed with 2.0 N HCl (25.0 mL). The organic layer was separated, dried and concentrated to give a yellow oil. The crude product was purified by Biotage with 0-15% methanol in CH₂Cl₂ to give 2.79 g (90%) product as a white solid. A small amount of sample was purified by HPLC under acidic condition to give the product as a TFA salt: ¹H NMR (400 MHz, DMSO-d₆) δ ppm 7.74-7.88 (m, 2H), 7.50-7.64 (m, 2H), 7.33 (s, 1H), 3.90 (dt, J=12.7, 3.5 Hz, 2H), 3.04-3.23 (m, 2H), 2.51-2.59 (m, 1H), 1.94 (dd, J=13.1, 3.7 Hz, 2H), 1.51-1.75 (m, 2H); ¹⁹F NMR (376 MHz, DMSO-d₆) δ ppm −74.87 (s); LCMS RT=5.841 min, m/z 367.0 [M+H⁺]; HRMS (ESI) m/z calcd for C₁₅H₁₆⁷⁹BrN₂O₂S [M+H⁺] 367.0116.

### Example 70

### 1-(4-(4-Bromophenyl)Thiazol-2-yl)-N-Methylpiperidine-4-Carboxamide (XJB01-077)

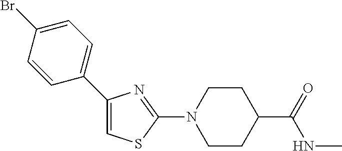 [SMILES: CNC(=O)C1CCN(c2nc(-c3ccc(Br)cc3)cs2)CC1]

The compound was prepared according to the general protocol B as a TFA salt: ¹H NMR (400 MHz, CHLOROFORM-d) δ ppm 7.60-7.71 (m, 2H), 7.47-7.57 (m, 2H), 6.72 (s, 1H), 5.78 (br. s., 1H), 4.15 (dt, J=13.3, 3.5 Hz, 2H), 3.10-3.30 (m, 2H), 2.84 (d, J=5.1 Hz, 3H), 2.41 (tt, J=11.1, 3.8 Hz, 1H), 1.95-2.05 (m, 2H), 1.81-1.95 (m, 2H); ¹⁹F NMR (376 MHz, CHLOROFORM-d) δ ppm −75.97 (s); LCMS RT=5.338 min, m/z 380.0 [M+H⁺]; HRMS (ESI) m/z calcd for C₁₆H₁₉⁷⁹BrN₃OS [M+H⁺] 380.0432.

### Example 71

### 1-(4-(4-Bromophenyl)Thiazol-2-yl)-N,N-Dimethylpiperidine-4-Carboxamide (XJB01-083)

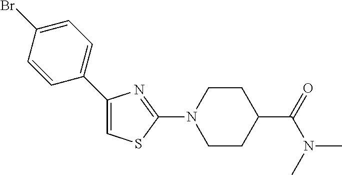 [SMILES: CN(C)C(=O)C1CCN(c2nc(-c3ccc(Br)cc3)cs2)CC1]

The compound was prepared according to the general protocol B as a TFA salt: ¹H NMR (400 MHz, CHLOROFORM-d) δ ppm 10.76 (br. s., 1H), 7.40-7.70 (m, 4H), 6.67 (s, 1H), 4.16 (dt, J=13.3, 3.9 Hz, 2H), 3.22-3.50 (m, 2H), 3.12 (s, 3H), 2.99 (s, 3H), 2.79-2.95 (m, 1H), 1.79-2.13 (m, 4H); ¹⁹F NMR (376 MHz, CHLOROFORM-d) δ ppm −76.00 (s); LCMS RT=5.705 min, m/z 394.0 [M+H⁺]; HRMS (ESI) m/z calcd for C₁₇H₂₁⁷⁹BrN₃OS [M+H⁺] 394.0589.

### Example 72

### 1-(4-(4-Bromophenyl)Thiazol-2-yl)-N-Phenylpiperidine-4-Carboxamide (XJB01-085)

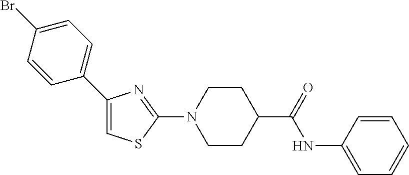 [SMILES: O=C(Nc1ccccc1)C1CCN(c2nc(-c3ccc(Br)cc3)cs2)CC1]

The compound was prepared according to the general protocol B as a TFA salt: ¹H NMR (400 MHz, CHLOROFORM-d) δ ppm 9.48 (br. s., 2H), 8.12 (s, 1H), 7.41-7.71 (m, 5H), 7.30 (t, J=7.8 Hz, 2H), 7.11 (t, J=7.2 Hz, 1H), 6.67 (s, 1H), 4.08-4.25 (m, 2H), 3.25-3.42 (m, 2H), 2.57-2.72 (m, 1H), 1.90-2.17 (m, 4H); ¹⁹F NMR (376 MHz, CHLOROFORM-d) δ ppm −75.82 (s); LCMS RT=6.676 min, m/z 442.0 [M+H⁺]; HRMS (ESI) m/z calcd for C₂₁H₂₁⁷⁹BrN₃OS [M+H⁺] 442.0589.

### Example 73

### N-Benzyl-1-(4-(4-Bromophenyl)Thiazol-2-yl)Piperidine-4-Carboxamide (XJB01-086)

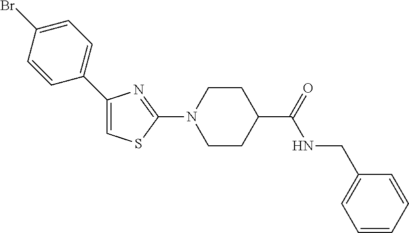 [SMILES: O=C(NCc1ccccc1)C1CCN(c2nc(-c3ccc(Br)cc3)cs2)CC1]

The compound was prepared according to the general protocol B as a TFA salt: ¹H NMR (400 MHz, CHLOROFORM-d) δ ppm 10.50 (br. s., 1H), 7.40-7.71 (m, 4H), 7.11-7.40 (m, 5H), 6.67 (s, 1H), 6.41 (t, J=5.5 Hz, 1H), 4.42 (d, J=5.9 Hz, 2H), 4.14 (dt, J=13.2, 3.4 Hz, 2H), 3.17-3.51 (m, 2H), 2.49 (tt, J=10.7, 3.9 Hz, 1H), 1.66-2.16 (m, 4H); ¹⁹F NMR (376 MHz, CHLOROFORM-d) δ ppm −75.92 (s); LCMS RT=6.431 min, m/z 456.0 [M+H⁺]; HRMS (ESI) m/z calcd for C₂₂H₂₃⁷⁹BrN₃OS [M+H⁺] 456.0745.

### Example 74

### (1-(4-(4-Bromophenyl)Thiazol-2-yl)Piperidin-4-yl)(Morpholino)Methanone (XJB01-087)

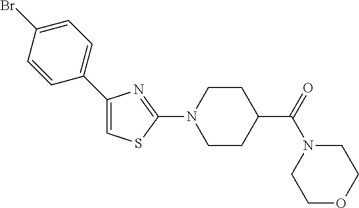 [SMILES: O=C(C1CCN(c2nc(-c3ccc(Br)cc3)cs2)CC1)N1CCOCC1]

The compound was prepared according to the general protocol B as a TFA salt: ¹H NMR (400 MHz, CHLOROFORM-d) δ ppm 7.60-7.67 (m, 2H), 7.58 (br. s., 1H), 7.49-7.54 (m, 2H), 6.71 (s, 1H), 4.15 (dt, J=13.3, 3.7 Hz, 2H), 3.71 (br. s., 4H), 3.60-3.68 (m, 2H), 3.49-3.60 (m, 2H), 3.28 (ddd, J=13.0, 11.4, 3.3 Hz, 2H), 2.73-2.78 (tt, J=10.5, 3.9 Hz, 1H), 1.83-2.10 (m, 4H); ¹⁹F NMR (376 MHz, CHLOROFORM-d) δ ppm −75.98 (s); LCMS RT=5.689 min, m/z 436.0 [M+H⁺]; HRMS (ESI) m/z calcd for C₁₉H₂₃⁷⁹BrN₃O₂S [M+H⁺] 436.0694.

### Example 75

### (1-(4-(4-Bromophenyl)Thiazol-2-yl)Piperidin-4-yl)(Piperidin-1-yl)Methanone (XJB01-088)

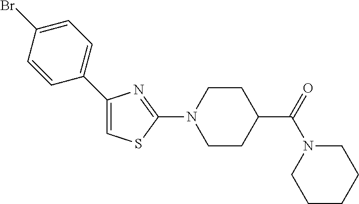 [SMILES: O=C(C1CCN(c2nc(-c3ccc(Br)cc3)cs2)CC1)N1CCCCC1]

The compound was prepared according to the general protocol B as a TFA salt: ¹H NMR (400 MHz, CHLOROFORM-d) δ ppm 7.58-7.69 (m, 2H), 7.49-7.57 (m, 2H), 6.68 (s, 1H), 5.73 (br. s., 1H), 4.16 (dt, J=13.2, 4.0 Hz, 2H), 3.53-3.64 (m, 2H), 3.45-3.52 (m, 2H), 3.35 (ddd, J=13.1, 11.2, 3.5 Hz, 2H), 2.84 (tt, J=10.2, 3.9 Hz, 1H), 1.84-2.06 (m, 4H), 1.48-1.75 (m, 6H); ¹⁹F NMR (376 MHz, CHLOROFORM-d) δ ppm −75.99 (s); LCMS RT=6.476 min, m/z 434.0 [M+H⁺]; HRMS (ESI) m/z calcd for C₂₀H₂₅⁷⁹BrN₃OS [M+H⁺] 434.0902.

### Example 76

### (1-(4-(4-Bromophenyl)Thiazol-2-yl)Piperidin-4-yl)(Piperazin-1-yl)Methanone (XJB01-089)

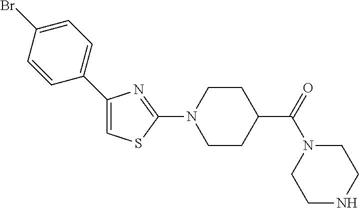 [SMILES: O=C(C1CCN(c2nc(-c3ccc(Br)cc3)cs2)CC1)N1CCNCC1]

The compound was prepared according to the general protocol B as a TFA salt: ¹H NMR (400 MHz, DMSO-d₆) δ ppm 8.84 (br. s., 2H), 7.70-7.89 (m, 2H), 7.52-7.65 (m, 2H), 7.34 (s, 1H), 3.88-4.05 (m, 2H), 3.76 (br. s., 2H), 3.65 (br. s., 2H), 3.01-3.25 (m, 6H), 2.94 (tt, J=11.1, 3.3 Hz, 1H), 1.70-1.85 (m, 2H), 1.52-1.71 (m, 2H); ¹⁹F NMR (376 MHz, DMSO-d₆) δ ppm −73.99 (s); LCMS RT=4.422 min, m/z 435.0 [M+H⁺]; HRMS (ESI) m/z calcd for C₁₉H₂₄⁷⁹BrN₄OS [M+H⁺] 435.0854.

### Example 77

### 1-(4-(4-Bromophenyl)Thiazol-2-yl)-N-Phenethylpiperidine-4-Carboxamide (XJB01-090)

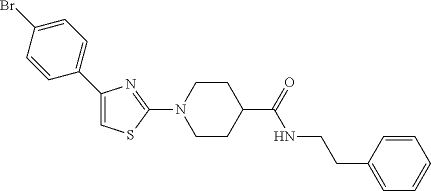 [SMILES: O=C(NCCc1ccccc1)C1CCN(c2nc(-c3ccc(Br)cc3)cs2)CC1]

The compound was prepared according to the general protocol B as a TFA salt: ¹H NMR (400 MHz, CHLOROFORM-d) δ ppm 7.62 (br. s., 1H), 7.57-7.61 (m, 2H), 7.51-7.56 (m, 2H), 7.29-7.36 (m, 2H), 7.22-7.27 (m, 1H), 7.15-7.22 (m, 2H), 6.68 (s, 1H), 5.76 (t, J=5.9 Hz, 1H), 4.12 (dt, J=13.3, 3.9 Hz, 2H), 3.52-3.59 (m, 2H), 3.29 (ddd, J=13.3, 11.2, 3.3 Hz, 2H), 2.84 (t, J=6.8 Hz, 2H), 2.39 (tt, J=10.8, 3.9 Hz, 1H), 1.93-2.02 (m, 2H), 1.80-1.93 (m, 2H); ¹⁹F NMR (376 MHz, CHLOROFORM-d) δ ppm −75.90 (s); LCMS RT=6.640 min, m/z 470.0 [M+H⁺]; HRMS (ESI) m/z calcd for C₂₃H₂₅⁷⁹BrN₃OS [M+H⁺] 470.0902.

### Example 78

### 1-(4-(4-Bromophenyl)Thiazol-2-yl)-N-(3-Phenylpropyl)Piperidine-4-Carboxamide (XJB01-091)

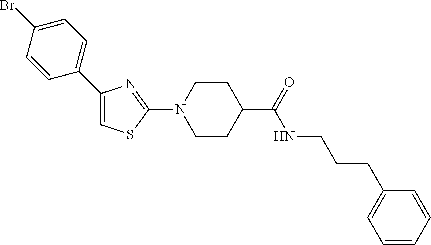 [SMILES: O=C(NCCCc1ccccc1)C1CCN(c2nc(-c3ccc(Br)cc3)cs2)CC1]

The compound was prepared according to the general protocol B as a TFA salt: ¹H NMR (400 MHz, CHLOROFORM-d) δ ppm 9.09 (br. s., 2H), 7.41-7.71 (m, 3H), 7.30 (t, J=7.2 Hz, 2H), 7.14-7.24 (m, 3H), 6.67 (s, 1H), 5.85 (t, J=5.5 Hz, 1H), 4.14 (dt, J=13.3, 3.7 Hz, 2H), 3.26-3.40 (m, 4H), 2.67 (t, J=7.4 Hz, 2H), 2.39 (tt, J=10.8, 3.9 Hz, 1H), 1.93-2.03 (m, 2H), 1.80-1.92 (m, 4H); ¹⁹F NMR (376 MHz, CHLOROFORM-d) δ ppm −75.91 (s); LCMS RT=6.832 min, m/z 484.0 [M+H⁺]; HRMS (ESI) m/z calcd for C₂₄H₂₇⁷⁹BrN₃OS [M+H⁺] 484.1058.

### Example 79

### 1-(4-(4-Bromophenyl)Thiazol-2-yl)-N-(4-Phenylbutyl)Piperidine-4-Carboxamide (XJB01-092)

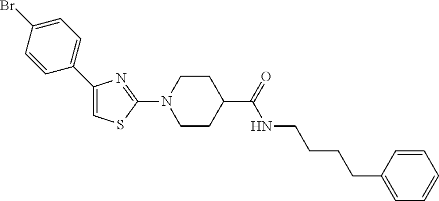 [SMILES: O=C(NCCCCc1ccccc1)C1CCN(c2nc(-c3ccc(Br)cc3)cs2)CC1]

The compound was prepared according to the general protocol B as a TFA salt: ¹H NMR (400 MHz, CHLOROFORM-d) δ ppm 7.46-7.65 (m, 4H), 7.27-7.32 (m, 2H), 7.14-7.22 (m, 3H), 6.67 (s, 1H), 5.85 (t, J=5.7 Hz, 1H), 4.16 (ddd, J=13.5, 3.9, 3.7 Hz, 2H), 3.23-3.41 (m, 4H), 2.64 (t, J=7.4 Hz, 2H), 2.46 (tt, J=10.9, 4.0 Hz, 1H), 2.00-2.09 (m, 2H), 1.86-1.99 (m, 2H), 1.60-1.71 (m, 2H), 1.50-1.60 (m, 2H); ¹⁹F NMR (376 MHz, CHLOROFORM-d) δ ppm −75.96 (s); LCMS RT=7.049 min, m/z 498.1 [M+H⁺]; HRMS (ESI) m/z calcd for C₂₅H₂₉⁷⁹BrN₃OS [M+H⁺] 498.1215.

### Example 80

### 1-(4-(4-Bromophenyl)Pyrimidin-2-yl)Piperidine-4-Carboxamide (XJB02-015)

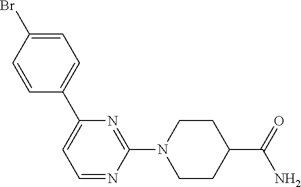 [SMILES: NC(=O)C1CCN(c2nccc(-c3ccc(Br)cc3)n2)CC1]

The compound was prepared according to the general protocol A as a TFA salt: ¹H NMR (400 MHz, DMSO-d₆) δ ppm 8.44 (d, J=5.1 Hz, 1H), 8.06-8.12 (m, 2H), 7.68-7.75 (m, 2H), 7.30 (br. s., 1H), 7.21 (d, J=5.1 Hz, 1H), 6.78 (br. s., 1H), 4.68-4.85 (m, 2H), 2.97 (td, J=12.8, 2.6 Hz, 2H), 2.31-2.47 (m, 1H), 1.80 (dd, J=13.3, 3.3 Hz, 2H), 1.50 (qd, J=12.4, 4.1 Hz, 2H); ¹⁹F NMR (376 MHz, DMSO-d₆) δ ppm −75.01 (s); LCMS RT=4.550 min, m/z 361.0 [M+H⁺]; HRMS (ESI) m/z calcd for C₁₆H₁₈⁷⁹BrN₄O [M+H⁺] 361.0664.

### Example 81

### 1-(4-(3-(Trifluoromethyl)Phenyl)Thiazol-2-yl)Piperidine-4-Carboxamide (XJB02-016)

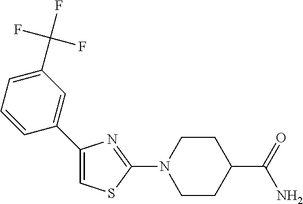 [SMILES: NC(=O)C1CCN(c2nc(-c3cccc(C(F)(F)F)c3)cs2)CC1]

The compound was prepared according to the general protocol A as a TFA salt: ¹H NMR (400 MHz, DMSO-d₆) δ ppm 8.02-8.32 (m, 2H), 7.57-7.81 (m, 2H), 7.50 (s, 1H), 7.33 (br. s., 1H), 6.83 (br. s., 1H), 3.98 (dt, J=12.7, 3.1 Hz, 2H), 3.08 (td, J=12.5, 2.9 Hz, 2H), 2.35 (tt, J=11.5, 3.6 Hz, 1H), 1.83 (dd, J=12.8, 2.6 Hz, 2H), 1.48-1.75 (m, 2H); ¹⁹F NMR (376 MHz, DMSO-d₆) δ ppm −61.13 (s), −74.84 (s); LCMS RT=5.463 min, m/z 356.1 [M+H⁺]; HRMS (ESI) m/z calcd for C₁₆H₁₇F₃N₃OS [M+H⁺] 356.1044.

### Example 82

### 1-(4-(4-Aminophenyl)Thiazol-2-yl)Piperidine-4-Carboxamide (XJB02-018)

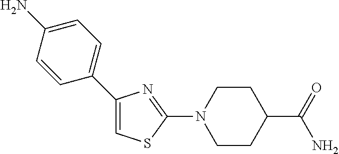 [SMILES: NC(=O)C1CCN(c2nc(-c3ccc(N)cc3)cs2)CC1]

The compound was prepared according to the general protocol A: ¹H NMR (400 MHz, DMSO-d₆) δ ppm 7.43-7.68 (m, 2H), 7.31 (s, 1H), 6.81 (s, 2H), 6.40-6.65 (m, 2H), 5.75 (s, 1H), 5.17 (s, 2H), 3.82-4.01 (m, 2H), 3.01 (td, J=12.7, 3.1 Hz, 2H), 2.22-2.38 (m, 1H), 1.80 (dd, J=13.4, 3.2 Hz, 2H), 1.55-1.71 (m, 1H); LCMS RT=2.906 min, m/z 303.1 [M+H⁺]; HRMS (ESI) m/z calcd for C₁₅H₁₉N₄OS [M+H⁺] 303.1280.

### Example 83

### 1-(4-(3,4-Dimethoxyphenyl)Thiazol-2-yl)Piperidine-4-Carboxamide (XJB02-020)

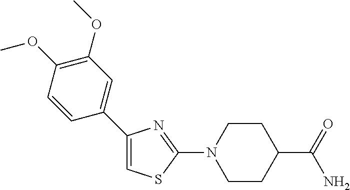 [SMILES: COc1ccc(-c2csc(N3CCC(C(N)=O)CC3)n2)cc1OC]

The compound was prepared according to the general protocol A as a TFA salt: ¹H NMR (400 MHz, DMSO-d₆) δ ppm 7.36-7.43 (m, 2H), 7.33 (br. s., 1H), 7.12 (s, 1H), 6.96 (d, J=9.0 Hz, 1H), 6.83 (br. s., 1H), 3.92-4.04 (m, 2H), 3.80 (s, 3H), 3.77 (s, 3H), 3.06 (td, J=12.5, 2.9 Hz, 2H), 2.36 (tt, J=11.5, 3.6 Hz, 1H), 1.83 (dd, J=13.2, 2.8 Hz, 2H), 1.62 (qd, J=12.4, 4.3 Hz, 2H); ¹⁹F NMR (376 MHz, DMSO-d₆) δ ppm −75.07 (s); LCMS RT=3.777 min, m/z 348.1 [M+H⁺]; HRMS (ESI) m/z calcd for C₁₇H₂₂N₃O₃S [M+H⁺] 348.1382.

### Example 84

### 1-(4-(3,4,5-Trimethoxyphenyl)Thiazol-2-yl)Piperidine-4-Carboxamide (XJB02-021)

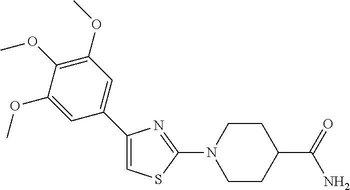 [SMILES: COc1cc(-c2csc(N3CCC(C(N)=O)CC3)n2)cc(OC)c1OC]

The compound was prepared according to the general protocol A as a TFA salt: ¹H NMR (400 MHz, DMSO-d₆) δ ppm 7.33 (br. s., 1H), 7.23 (s, 1H), 7.14 (s, 2H), 6.84 (br. s., 1H), 3.90-4.06 (m, 2H), 3.82 (s, 6H), 3.68 (s, 3H), 3.06 (td, J=12.5, 2.8 Hz, 2H), 2.36 (tt, J=11.5, 3.6 Hz, 1H), 1.83 (dd, J=13.2, 2.8 Hz, 2H), 1.53-1.72 (m, 2H); ¹⁹F NMR (376 MHz, DMSO-d₆) δ ppm −75.13 (s); LCMS RT=4.061 min, m/z 378.1 [M+H⁺]; HRMS (ESI) m/z calcd for C₁₈H₂₄N₃O₄S [M+H⁺] 378.1488.

### Example 85

### 1-(4-(3,5-Dimethoxyphenyl)Thiazol-2-yl)Piperidine-4-Carboxamide (XJB02-022)

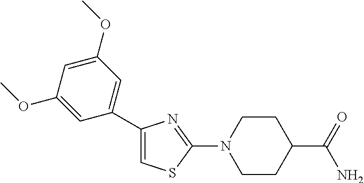 [SMILES: COc1cc(OC)cc(-c2csc(N3CCC(C(N)=O)CC3)n2)c1]

The compound was prepared according to the general protocol A as a TFA salt: ¹H NMR (400 MHz, DMSO-d₆) δ ppm 7.32 (br. s., 1H), 7.29 (s, 1H), 7.01 (d, J=2.3 Hz, 2H), 6.83 (br. s., 1H), 6.44 (t, J=2.2 Hz, 1H), 3.88-4.02 (m, 2H), 3.77 (s, 6H), 3.06 (td, J=12.5, 2.8 Hz, 2H), 2.35 (tt, J=11.4, 3.6 Hz, 1H), 1.82 (dd, J=13.0, 2.6 Hz, 2H), 1.54-1.70 (m, 2H); ¹⁹F NMR (376 MHz, DMSO-d₆) δ ppm −75.07 (s); LCMS RT=4.458 min, m/z 348.1 [M+H⁺]; HRMS (ESI) m/z calcd for C₁₇H₂₂N₃O₃S [M+H⁺] 348.1382.

### Example 86

### 1-(4-(4-(Methylthio)Phenyl)Thiazol-2-yl)Piperidine-4-Carboxamide (XJB02-023)

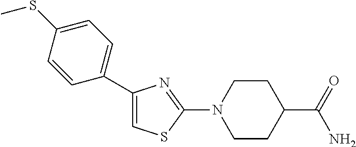 [SMILES: CSc1ccc(-c2csc(N3CCC(C(N)=O)CC3)n2)cc1]

The compound was prepared according to the general protocol A as a TFA salt: ¹H NMR (400 MHz, DMSO-d₆) δ ppm 7.74-7.84 (m, 2H), 7.32 (br. s., 1H), 7.23-7.29 (m, 2H), 7.21 (s, 1H), 6.82 (br. s., 1H), 3.96 (ddd, J=12.7, 3.6, 3.4 Hz, 2H), 3.06 (td, J=12.6, 3.0 Hz, 2H), 2.49 (s, 3H), 2.26-2.42 (m, 1H), 1.82 (dd, J=13.5, 3.3 Hz, 2H), 1.53-1.70 (m, 2H); ¹⁹F NMR (376 MHz, DMSO-d₆) δ ppm −74.69 (s); LCMS RT=4.653 min, m/z 334.1 [M+H⁺]; HRMS (ESI) m/z calcd for C₁₆H₂₀N₃OS₂ [M+H⁺] 334.1048.

### Example 87

### 1-(5-(4-Bromophenyl)Thiazol-2-yl)Piperidine-4-Carboxamide (XJB02-024)

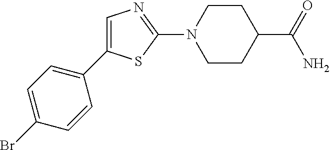 [SMILES: NC(=O)C1CCN(c2ncc(-c3ccc(Br)cc3)s2)CC1]

The compound was prepared according to the general protocol A as a TFA salt: ¹H NMR (400 MHz, DMSO-d₆) δ ppm 7.66 (s, 1H), 7.50-7.57 (m, 2H), 7.38-7.45 (m, 2H), 7.32 (br. s., 1H), 6.83 (br. s., 1H), 3.87-3.97 (m, 2H), 3.10 (td, J=12.7, 2.9 Hz, 2H), 2.30-2.41 (m, 1H), 1.81 (dd, J=13.4, 2.4 Hz, 2H), 1.54-1.69 (m, 2H); ¹⁹F NMR (376 MHz, DMSO-d₆) δ ppm −74.75 (s); LCMS RT=4.408 min, m/z 366.0 [M+H⁺]; HRMS (ESI) m/z calcd for C₁₅H₁₇⁷⁹BrN₃OS [M+H⁺] 366.0276.

### Example 88

### 1-(5-(3-(Dimethylamino)Phenyl)Thiazol-2-yl)Piperidine-4-Carboxamide (XJB02-026)

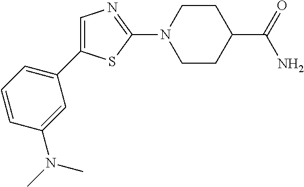 [SMILES: CN(C)c1cccc(-c2cnc(N3CCC(C(N)=O)CC3)s2)c1]

The compound was prepared according to the general protocol A as a TFA salt: ¹H NMR (400 MHz, DMSO-d₆) δ ppm 7.64 (s, 1H), 7.33 (br. s., 1H), 7.14-7.26 (m, 1H), 6.85 (d, J=6.3 Hz, 3H), 6.72 (d, J=7.8 Hz, 1H), 3.92 (dt, J=13.1, 3.3 Hz, 2H), 3.07-3.22 (m, 2H), 2.95 (s, 6H), 2.38 (tt, J=11.3, 3.7 Hz, 1H), 1.82 (dd, J=12.9, 2.5 Hz, 2H), 1.52-1.74 (m, 2H); ¹⁹F NMR (376 MHz, DMSO-d₆) δ ppm −74.87 (s); LCMS RT=3.199 min, m/z 331.1 [M+H⁺]; HRMS (ESI) m/z calcd for C₁₇H₂₃N₄OS [M+H⁺] 331.1593.

### Example 89

### 1-(5-(3-(Dimethylamino)Phenyl)Pyrimidin-2-yl)Piperidine-4-Carboxamide (XJB02-027)

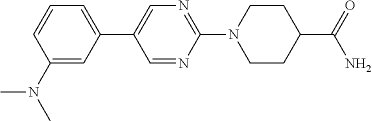 [SMILES: CN(C)c1cccc(-c2cnc(N3CCC(C(N)=O)CC3)nc2)c1]

The compound was prepared according to the general protocol A as a TFA salt: ¹H NMR (400 MHz, DMSO-d₆) δ ppm 8.69 (s, 2H), 7.31 (t, J=7.9 Hz, 2H), 6.97-7.17 (m, 2H), 6.63-6.96 (m, 2H), 4.68 (dt, J=13.2, 3.0 Hz, 2H), 3.00 (s, 6H), 2.91-2.99 (m, 2H), 2.42 (tt, J=11.5, 3.7 Hz, 1H), 1.77 (dd, J=12.7, 2.5 Hz, 2H), 1.49 (qd, J=12.4, 4.2 Hz, 2H); ¹⁹F NMR (376 MHz, DMSO-d₆) δ ppm −74.92 (s); LCMS RT=3.341 min, m/z 326.1 [M+H⁺]; HRMS (ESI) m/z calcd for C₁₈H₂₄N₅O [M+H⁺] 326.1981.

### Example 90

### 1-(4-(3-(Dimethylamino)Phenyl)Pyrimidin-2-yl)Piperidine-4-Carboxamide (XJB02-028)

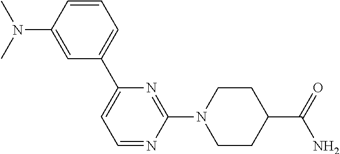 [SMILES: CN(C)c1cccc(-c2ccnc(N3CCC(C(N)=O)CC3)n2)c1]

The compound was prepared according to the general protocol A as a TFA salt: ¹H NMR (400 MHz, DMSO-d₆) δ ppm 8.40 (d, J=5.3 Hz, 1H), 7.38-7.51 (m, 2H), 7.33 (t, J=7.9 Hz, 1H), 7.29 (br. s., 1H), 7.16 (d, J=5.3 Hz, 1H), 6.94 (d, J=9.0 Hz, 1H), 6.78 (br. s., 1H), 4.65-4.86 (m, 2H), 2.99 (s, 6H), 2.90-2.98 (m, 2H), 2.41 (tt, J=11.6, 4.1 Hz, 1H), 1.79 (dd, J=13.1, 3.3 Hz, 2H), 1.51 (qd, J=12.3, 3.9 Hz, 2H); ¹⁹F NMR (376 MHz, DMSO-d₆) δ ppm −74.71 (s); LCMS RT=3.327 min, m/z 326.2 [M+H⁺]; HRMS (ESI) m/z calcd for C₁₈H₂₄N₅O [M+H⁺] 326.1981.

### Example 91

### 1-(4-(4-Isopropoxyphenyl)Thiazol-2-yl)Piperidine-4-Carboxamide (XJB02-0361

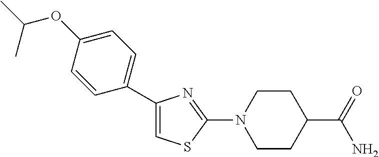 [SMILES: CC(C)Oc1ccc(-c2csc(N3CCC(C(N)=O)CC3)n2)cc1]

The compound was prepared according to the general protocol A: ¹H NMR (400 MHz, DMSO-d₆) δ ppm 7.63-7.89 (m, 2H), 7.32 (br. s., 1H), 7.05 (s, 1H), 6.87-6.96 (m, 2H), 6.81 (br. s., 1H), 4.63 (dt, J=12.1, 6.1 Hz, 1H), 3.95 (dt, J=12.7, 3.2 Hz, 2H), 3.04 (td, J=12.5, 2.7 Hz, 2H), 2.35 (tt, J=11.5, 3.6 Hz, 1H), 1.81 (dd, J=13.2, 2.8 Hz, 2H), 1.55-1.68 (m, 2H), 1.28 (s, 3H), 1.26 (s, 3H); LCMS RT=4.640 min, m/z 346.1 [M+H⁺]; HRMS (ESI) m/z calcd for C₁₈H₂₄N₃O₂S [M+H⁺] 346.1589.

### Example 92

### 1-(4-(4-Phenoxyphenyl)Thiazol-2-yl)Piperidine-4-Carboxamide (XJB02-037)

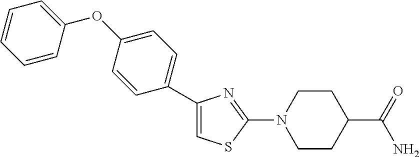 [SMILES: NC(=O)C1CCN(c2nc(-c3ccc(Oc4ccccc4)cc3)cs2)CC1]

The compound was prepared according to the general protocol A: ¹H NMR (400 MHz, DMSO-d₆) δ ppm 7.81-7.91 (m, 2H), 7.35-7.47 (m, 2H), 7.32 (br. s., 1H), 7.12-7.21 (m, 2H), 6.98-7.06 (m, 4H), 6.82 (br. s., 1H), 3.96 (dt, J=12.8, 3.4 Hz, 2H), 3.05 (td, J=12.6, 3.0 Hz, 2H), 2.35 (tt, J=11.4, 4.0 Hz, 1H), 1.82 (dd, J=13.3, 3.1 Hz, 2H), 1.63 (qd, J=12.4, 4.3 Hz, 2H); LCMS RT=5.337 min, m/z 380.1 [M+H⁺]; HRMS (ESI) m/z calcd for C₂₁H₂₂N₃O₂S [M+H⁺] 380.1433.

### Example 93

### 1-(4-(2,3-Dihydrobenzofuran-6-yl)Thiazol-2-yl)Piperidine-4-Carboxamide (XJB02-038)

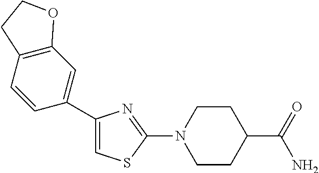 [SMILES: NC(=O)C1CCN(c2nc(-c3ccc4c(c3)OCC4)cs2)CC1]

The compound was prepared according to the general protocol A: ¹H NMR (400 MHz, DMSO-d₆) δ ppm 7.72 (d, J=1.4 Hz, 1H), 7.52-7.63 (m, 1H), 7.32 (br. s., 1H), 7.01 (s, 1H), 6.82 (br. s., 1H), 6.75 (d, J=8.4 Hz, 1H), 4.54 (t, J=8.7 Hz, 2H), 3.95 (dt, J=13.0, 3.4 Hz, 2H), 3.20 (t, J=8.8 Hz, 2H), 3.03 (td, J=12.5, 2.9 Hz, 2H), 2.29-2.40 (m, 1H), 1.81 (dd, J=13.6, 3.2 Hz, 2H), 1.50-1.70 (m, 2H); LCMS RT=3.944 min, m/z 330.1 [M+H⁺]; HRMS (ESI) m/z calcd for C₁₇H₂₀N₃O₂S [M+H⁺] 330.1276.

### Example 94

### 1-(4-(4-(Benzyloxy)Phenyl)Thiazol-2-yl)Piperidine-4-Carboxamide (XJB02-039)

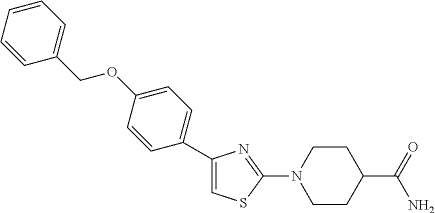 [SMILES: NC(=O)C1CCN(c2nc(-c3ccc(OCc4ccccc4)cc3)cs2)CC1]

The compound was prepared according to the general protocol A as a TFA salt: ¹H NMR (400 MHz, DMSO-d₆) δ ppm 7.74-7.81 (m, 2H), 7.27-7.51 (m, 6H), 7.07 (s, 1H), 6.97-7.05 (m, 2H), 6.82 (br. s., 1H), 5.13 (s, 2H), 3.95 (dt, J=13.0, 3.6 Hz, 2H), 3.05 (td, J=12.6, 2.9 Hz, 2H), 2.26-2.41 (m, 1H), 1.77-1.88 (m, 2H), 1.54-1.69 (m, 2H); ¹⁹F NMR (376 MHz, DMSO-d₆) δ ppm −74.49 (s); LCMS RT=5.085 min, m/z 394.1 [M+H⁺]; HRMS (ESI) m/z calcd for C₂₂H₂₄N₃O₂S [M+H⁺] 394.1589.

### Example 95

### 1-(4-(4-(Trifluoromethoxy)Phenyl)Thiazol-2-yl)Piperidine-4-Carboxamide (XJB02-040)

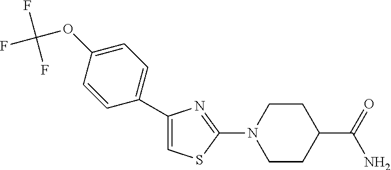 [SMILES: NC(=O)C1CCN(c2nc(-c3ccc(OC(F)(F)F)cc3)cs2)CC1]

The compound was prepared according to the general protocol A: ¹H NMR (400 MHz, DMSO-d₆) δ ppm 7.89-8.04 (m, 2H), 7.37 (dd, J=9.0, 1.0 Hz, 2H), 7.33 (s, 1H), 7.32 (br. s., 1H), 6.82 (br. s., 1H), 3.96 (dt, J=13.1, 3.5 Hz, 2H), 3.07 (td, J=12.5, 2.7 Hz, 2H), 2.26-2.43 (m, 1H), 1.82 (dd, J=12.8, 2.6 Hz, 2H), 1.62 (qd, J=12.3, 3.9 Hz, 2H); ¹⁹F NMR (376 MHz, DMSO-d₆) δ ppm −56.77 (s); LCMS RT=5.491 min, m/z 372.1 [M+H⁺]; HRMS (ESI) m/z calcd for C₁₆H₁₇F₃N₃O₂S [M+H⁺] 372.0994.

### Example 96

### 1-(4-(4-Ethoxyphenyl)Thiazol-2-yl)Piperidine-4-Carboxamide (XJB02-041)

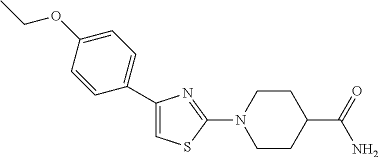 [SMILES: CCOc1ccc(-c2csc(N3CCC(C(N)=O)CC3)n2)cc1]

The compound was prepared according to the general protocol A: ¹H NMR (400 MHz, DMSO-d₆) δ ppm 7.70-7.83 (m, 2H), 7.32 (br. s., 1H), 7.06 (s, 1H), 6.86-6.96 (m, 2H), 6.82 (br. s., 1H), 4.04 (q, J=6.8 Hz, 2H), 3.95 (ddd, J=12.6, 3.6, 3.2 Hz, 2H), 3.04 (td, J=12.3, 2.9 Hz, 2H), 2.28-2.42 (m, 1H), 1.74-1.90 (m, 2H), 1.52-1.69 (m, 2H), 1.33 (t, J=6.9 Hz, 3H); LCMS RT=4.360 min, m/z 332.1 [M+H⁺]; HRMS (ESI) m/z calcd for C₁₇H₂₂N₃O₂S [M+H⁺] 332.1433.

### Example 97

### 1-(4-(4-(4-Methylpiperazin-1-yl)Phenyl)Thiazol-2-yl)Piperidine-4-Carboxamide (XJB02-042)

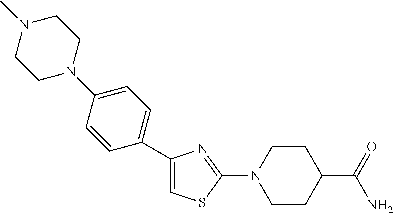 [SMILES: CN1CCN(c2ccc(-c3csc(N4CCC(C(N)=O)CC4)n3)cc2)CC1]

The compound was prepared according to the general protocol A as a TFA salt: ¹H NMR (400 MHz, DMSO-d₆) δ ppm 9.67 (br. s., 1H), 7.74 (d, J=7.0 Hz, 2H), 7.32 (br. s., 1H), 7.06 (d, J=1.6 Hz, 1H), 7.01 (d, J=7.4 Hz, 2H), 6.82 (br. s., 1H), 3.83-4.02 (m, 4H), 3.53 (d, J=12.5 Hz, 2H), 3.10-3.23 (m, 2H), 2.91-3.10 (m, 4H), 2.87 (br. s., 3H), 2.30-2.42 (m, 1H), 1.81 (d, J=12.9 Hz, 2H), 1.53-1.71 (m, 2H); ¹⁹F NMR (376 MHz, DMSO-d₆) δ ppm −74.49 (s); LCMS RT=3.032 min, m/z 386.1 [M+H⁺]; HRMS (ESI) m/z calcd for C₂₀H₂₈N₅OS [M+H⁺] 386.2015.

### Example 98

### 1-(4-(4-Tert-Butylphenyl)Thiazol-2-yl)Piperidine-4-Carboxamide (XJB02-043)

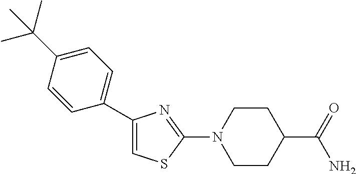 [SMILES: CC(C)(C)c1ccc(-c2csc(N3CCC(C(N)=O)CC3)n2)cc1]

The compound was prepared according to the general protocol A: ¹H NMR (400 MHz, DMSO-d₆) δ ppm 7.65-7.85 (m, 2H), 7.35-7.45 (m, 2H), 7.32 (br. s., 1H), 7.15 (s, 1H), 6.82 (br. s., 1H), 3.96 (dt, J=12.8, 3.5 Hz, 2H), 3.05 (td, J=12.6, 3.0 Hz, 2H), 2.20-2.31 (tt, J=11.8, 3.6 Hz, 1H), 1.82 (dd, J=13.2, 3.0 Hz, 2H), 1.52-1.74 (m, 2H), 1.29 (s, 9H); LCMS RT=5.300 min, m/z 344.1 [M+H⁺]; HRMS (ESI) m/z calcd for C₁₉H₂₆N₃OS [M+H⁺] 344.1797.

### Example 99

### 1-(4-(3,4-Dihydro-2H-Benzo[B][1,4]Dioxepin-7-yl)Thiazol-2-yl)Piperidine-4-Carboxamide (XJB02-044)

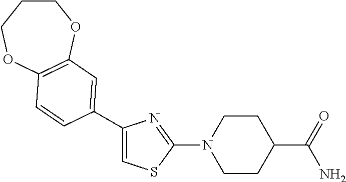 [SMILES: NC(=O)C1CCN(c2nc(-c3ccc4c(c3)OCCCO4)cs2)CC1]

The compound was prepared according to the general protocol A as a TFA salt: ¹H NMR (400 MHz, DMSO-d₆) δ ppm 7.45 (d, J=2.0 Hz, 1H), 7.40-7.44 (m, 1H), 7.32 (br. s., 1H), 7.14 (s, 1H), 6.96 (d, J=8.2 Hz, 1H), 6.82 (br. s., 1H), 4.13 (td, J=5.4, 3.3 Hz, 4H), 3.94 (dt, J=12.9, 3.3 Hz, 2H), 3.05 (td, J=12.5, 3.0 Hz, 2H), 2.34 (tt, J=11.5, 4.0 Hz, 1H), 2.11 (quin, J=5.4 Hz, 2H), 1.81 (dd, J=13.3, 2.9 Hz, 2H), 1.62 (qd, J=12.4, 4.5 Hz, 2H); ¹⁹F NMR (376 MHz, DMSO-d₆) δ ppm −74.87 (s); LCMS RT=4.211 min, m/z 360.1 [M+H⁺]; HRMS (ESI) m/z calcd for C₁₈H₂₂N₃O₃S [M+H⁺] 360.1382.

### Example 100

### 1-(4-(4-(Dimethylamino)Phenyl)Thiazol-2-yl)Piperidine-4-Carboxamide (XJB02-046)

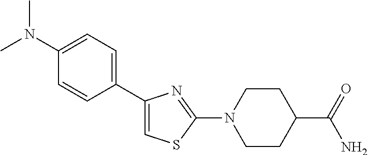 [SMILES: CN(C)c1ccc(-c2csc(N3CCC(C(N)=O)CC3)n2)cc1]

The compound was prepared according to the general protocol A as a TFA salt: ¹H NMR (400 MHz, DMSO-d₆) δ ppm 7.70 (d, J=8.8 Hz, 2H), 7.32 (br. s., 1H), 6.97 (s, 1H), 6.82 (br. s., 3H), 3.95 (dt, J=12.8, 3.4 Hz, 2H), 3.06 (td, J=12.5, 2.9 Hz, 2H), 2.96 (s, 6H), 2.35 (tt, J=11.3, 3.6 Hz, 1H), 1.82 (dd, J=13.3, 2.9 Hz, 2H), 1.62 (qd, J=12.4, 4.2 Hz, 2H); ¹⁹F NMR (376 MHz, DMSO-d₆) δ ppm −74.84 (s); LCMS RT=3.358 min, m/z 331.1 [M+H⁺]; HRMS (ESI) m/z calcd for C₁₇H₂₃N₄OS [M+H⁺] 331.1593.

### Example 101

### 4-(4-Bromophenyl)-2-(Methylsulfonyl)Thiazole (XJB02-049)

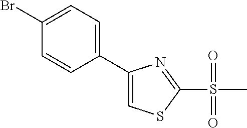 [SMILES: CS(=O)(=O)c1nc(-c2ccc(Br)cc2)cs1]

A mixture of 4-bromo-2-(methylthio)thiazole (1.00 g, 4.76 mmol), 4-bromophenylboronic acid (1.15 g, 5.71 mmol) and tetrakis(triphenylphosphine)palladium (275 mg, 0.238 mmol) in CH₃CN (6.00 mL) and 2.0 M Na₂CO₃ aqueous solution (2.00 mL) was heated in μW at 100° C. for 1 h. The reaction mixture was diluted with CH₂Cl₂, washed with H₂O. The organic layer was separated and dried with Na₂SO₄, concentrated in vacum to give yellow foam. The crude product was purified with Biotage with 0-50% CH₂Cl₂ in hexanes to give 804 mg (59%) 4-(4-bromophenyl)-2-(methylthio)thiazole which was used directly in the next reaction without further purification.

A solution of 4-(4-bromophenyl)-2-(methylthio)thiazole (700 mg, 2.446 mmol) in CH₂Cl₂ (20.0 mL) was treated with MCPBA (2.11 g, 12.2 mmol). The mixture was stirred at room temperature for 4 h. The reaction mixture was diluted with CH₂Cl₂, washed the saturated Na₂CO₃ solution. The organic layer was separated, dried Na₂SO₄, and concentrated as white solid. The crude product was purified with Biotage using 0-80% EtOAc in hexanes to give light yellow foam 826 mg, while the LCMS data showed two peaks. The sample was further purified by HPLC under acidic condition to give 195 mg (25%) product as a light yellow solid: ¹H NMR (400 MHz, CHLOROFORM-d) δ ppm 7.83 (s, 1H), 7.79-7.83 (m, 2H), 7.58-7.63 (m, 2H), 3.39 (s, 3H); LCMS RT=5.864 min, m/z 317.9 [M+H⁺]; HRMS (ESI) m/z calcd for C₁₀H₉⁷⁹BrNO₂S₂ [M+H⁺] 317.9258.

### Example 102

### 1-(4-(4-Bromophenyl)Thiazol-2-yl)Piperidine-4-Carbonitrile (XJB02-052)

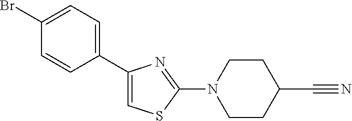 [SMILES: N#CC1CCN(c2nc(-c3ccc(Br)cc3)cs2)CC1]

The compound was prepared according to the general protocol A: ¹H NMR (400 MHz, CHLOROFORM-d) δ ppm 7.66-7.76 (m, 2H), 7.46-7.54 (m, 2H), 6.80 (s, 1H), 3.80 (ddd, J=13.4, 7.3, 3.9 Hz, 2H), 3.54 (ddd, J=13.4, 7.5, 3.8 Hz, 2H), 2.83-3.01 (m, 1H), 1.89-2.16 (m, 4H); LCMS RT=6.628 min, m/z 348.0 [M+H⁺]; HRMS (ESI) m/z calcd for C₁₅H₁₅⁷⁹BrN₃S [M+H⁺] 348.0170.

### Example 103

### Tert-Butyl 1-(4-(4-Bromophenyl)Thiazol-2-yl)Piperidin-4-Ylcarbamate (XJB02-053)

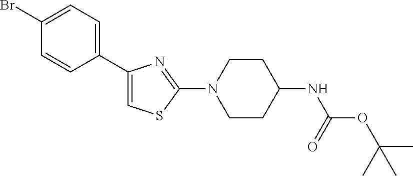 [SMILES: CC(C)(C)OC(=O)NC1CCN(c2nc(-c3ccc(Br)cc3)cs2)CC1]

The compound was prepared according to the general protocol A as a TFA salt: ¹H NMR (400 MHz, DMSO-d₆) δ ppm 7.74-7.85 (m, 2H), 7.50-7.63 (m, 2H), 7.31 (s, 1H), 6.90 (d, J=7.4 Hz, 1H), 3.89 (dt, J=13.2, 3.7 Hz, 2H), 3.53 (br. s., 1H), 3.05-3.19 (m, 2H), 1.82 (dd, J=13.5, 3.5 Hz, 2H), 1.40-1.55 (m, 2H), 1.39 (s, 9H); ¹⁹F NMR (376 MHz, DMSO-d₆) δ ppm −74.56 (s); LCMS RT=6.994 min, m/z 438.0 [M+H⁺]; HRMS (ESI) m/z calcd for C₁₉H₂₅⁷⁹BrN₃O₂S [M+H⁺] 438.0851.

### Example 104

### 1-(2-(3-(Dimethylamino)Phenyl)Pyrimidin-4-yl)Piperidine-4-Carboxamide (XJB02-055)

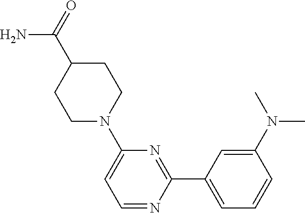 [SMILES: CN(C)c1cccc(-c2nccc(N3CCC(C(N)=O)CC3)n2)c1]

The compound was prepared according to the general protocol A: ¹H NMR (400 MHz, DMSO-d₆) δ ppm 8.27 (d, J=6.3 Hz, 1H), 7.73 (dd, J=2.6, 1.5 Hz, 1H), 7.59-7.67 (m, 1H), 7.30 (br. s., 1H), 7.26 (t, J=8.0 Hz, 1H), 6.84 (ddd, J=8.3, 2.7, 0.9 Hz, 1H), 6.80 (br. s., 1H), 6.76 (d, J=6.3 Hz, 1H), 4.50 (d, J=9.6 Hz, 2H), 2.97-3.06 (m, 2H), 2.95 (s, 6H), 2.43 (tt, J=11.3, 3.8 Hz, 1H), 1.81 (dd, J=13.4, 3.2 Hz, 2H), 1.43-1.61 (m, 2H); LCMS RT=3.225 min, m/z 326.1 [M+H⁺]; HRMS (ESI) m/z calcd for C₁₈H₂₄N₅O [M+H⁺] 326.1981.

### Example 105

### 1-(4-(4-Morpholinophenyl)Thiazol-2-yl)Piperidine-4-Carboxamide (XJB02-057)

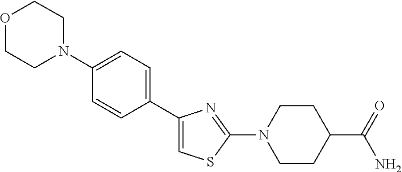 [SMILES: NC(=O)C1CCN(c2nc(-c3ccc(N4CCOCC4)cc3)cs2)CC1]

The compound was prepared according to the general protocol A as a TFA salt: ¹H NMR (400 MHz, DMSO-d₆) δ ppm 7.55-7.85 (m, 2H), 7.33 (br. s., 1H), 7.02 (s, 1H), 6.92-7.00 (m, 2H), 6.83 (br. s., 1H), 3.90-4.05 (m, 2H), 3.68-3.80 (m, 4H), 3.12-3.20 (m, 4H), 3.07 (td, J=12.5, 2.8 Hz, 2H), 2.36 (tt, J=11.5, 3.7 Hz, 1H), 1.82 (dd, J=12.9, 2.7 Hz, 2H), 1.56-1.70 (m, 2H); ¹⁹F NMR (376 MHz, DMSO-d₆) δ ppm −75.02 (s); LCMS RT=3.749 min, m/z 373.1 [M+H⁺]; HRMS (ESI) m/z calcd for C₁₉H₂₅N₄O₂S [M+H⁺] 373.1698.

### Example 106

### 1-(4-(4-Isobutoxyphenyl)Thiazol-2-yl)Piperidine-4-Carboxamide (XJB02-060)

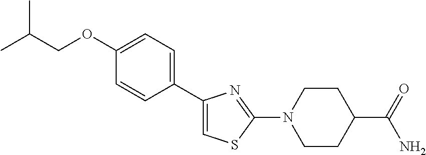 [SMILES: CC(C)COc1ccc(-c2csc(N3CCC(C(N)=O)CC3)n2)cc1]

The compound was prepared according to the general protocol A as a TFA salt: ¹H NMR (400 MHz, DMSO-d₆) δ ppm 7.60-7.88 (m, 2H), 7.32 (br. s., 1H), 7.06 (s, 1H), 6.89-6.97 (m, 2H), 6.82 (br. s., 1H), 3.95 (dt, J=12.8, 3.1 Hz, 2H), 3.76 (d, J=6.5 Hz, 2H), 3.05 (td, J=12.5, 2.8 Hz, 2H), 2.35 (tt, J=11.4, 3.6 Hz, 1H), 1.95-2.09 (m, 1H), 1.82 (dd, J=13.3, 2.9 Hz, 2H), 1.54-1.69 (m, 2H), 0.99 (d, J=6.8 Hz, 6H); ¹⁹F NMR (376 MHz, DMSO-d₆) δ ppm −74.92 (s); LCMS RT=5.191 min, m/z 360.2 [M+H⁺]; HRMS (ESI) m/z calcd for C₁₉H₂₆N₃O₂S [M+H⁺] 360.1746.

### Example 107

### 1-(4(3-Isopropoxyphenyl)Thiazol-2-yl)Piperidine-4-Carboxamide (XJB02-061)

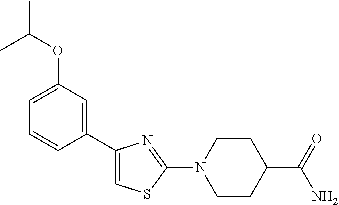 [SMILES: CC(C)Oc1cccc(-c2csc(N3CCC(C(N)=O)CC3)n2)c1]

The compound was prepared according to the general protocol A: ¹H NMR (400 MHz, DMSO-d₆) δ ppm 7.35-7.42 (m, 2H), 7.32 (br. s., 1H), 7.23-7.29 (m, 2H), 6.79-6.88 (m, 2H), 4.65 (spt, J=6.0 Hz, 1H), 3.96 (dt, J=12.7, 3.0 Hz, 2H), 3.05 (td, J=12.5, 2.9 Hz, 2H), 2.35 (tt, J=11.5, 3.5 Hz, 1H), 1.82 (dd, J=13.1, 2.7 Hz, 2H), 1.63 (qd, J=12.4, 4.3 Hz, 2H), 1.27 (d, J=6.1 Hz, 6H); LCMS RT=4.914 min, m/z 346.1 [M+H⁺]; HRMS (ESI) m/z calcd for C₁₈H₂₄N₃O₂S [M+H⁺] 346.1589.

### Example 108

### 4-(4-Bromophenyl)-2-(Piperazin-1-yl)Thiazole (XJB02-062)

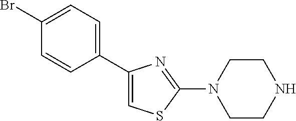 [SMILES: Brc1ccc(-c2csc(N3CCNCC3)n2)cc1]

The compound was prepared according to the general protocol A: ¹H NMR (400 MHz, DMSO-d₆) δ ppm 7.73-7.89 (m, 2H), 7.51-7.63 (m, 2H), 7.32 (s, 1H), 3.34-3.45 (m, 4H), 2.73-2.87 (m, 4H); LCMS RT=4.411 min, m/z 324.0 [M+H⁺]; HRMS (ESI) m/z calcd for C₁₃H₁₅⁷⁹BrN₃S [M+H⁺] 324.0170.

### Example 109

### 1-(4-(4-Carbamoylphenyl)Thiazol-2-yl)Piperidine-4-Carboxamide (XJB02-066)

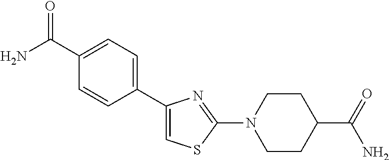 [SMILES: NC(=O)c1ccc(-c2csc(N3CCC(C(N)=O)CC3)n2)cc1]

The compound was prepared according to the general protocol A: ¹H NMR (400 MHz, DMSO-d₆) δ ppm 7.79-8.02 (m, 5H), 7.40 (s, 1H), 7.33 (br. s., 2H), 6.83 (br. s., 1H), 3.92-4.11 (m, 2H), 3.07 (td, J=12.5, 2.8 Hz, 2H), 2.36 (tt, J=11.5, 3.5 Hz, 1H), 1.83 (dd, J=13.0, 2.8 Hz, 2H), 1.63 (qd, J=12.4, 4.3 Hz, 2H); LCMS RT=3.352 min, m/z 331.1 [M+H⁺]; HRMS (ESI) m/z calcd for C₁₆H₁₉N₄O₂S [M+H⁺] 331.1229.

### Example 110

### 1-(4-(4-(4-Bromophenyl)Thiazol-2-yl)Piperazin-1-yl)Ethanone (XJB02-072)

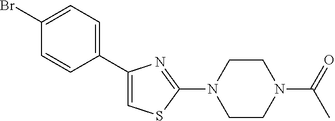 [SMILES: CC(=O)N1CCN(c2nc(-c3ccc(Br)cc3)cs2)CC1]

The compound was prepared according to the general protocol C as a TFA salt: ¹H NMR (400 MHz, CHLOROFORM-d) δ ppm 7.64-7.74 (m, 2H), 7.47-7.55 (m, 2H), 6.82 (s, 1H), 3.77-3.92 (m, 2H), 3.66 (br. s., 4H), 3.48-3.58 (m, 2H), 2.20 (s, 3H); ¹⁹F NMR (376 MHz, DMSO-d₆) δ ppm −76.04 (s); LCMS RT=5.892 min, m/z 366.0 [M+H⁺]; HRMS (ESI) m/z calcd for C₁₅H₁₇⁷⁹BrN₃OS [M+H⁺] 366.0276.

### Example 111

### 1-(4-(4-Bromophenyl)Thiazol-2-yl)Piperidin-4-Amine (XJB02-073)

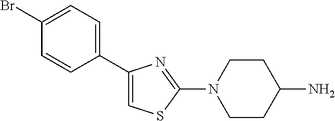 [SMILES: NC1CCN(c2nc(-c3ccc(Br)cc3)cs2)CC1]

A solution of tert-butyl 1-(4-(4-bromophenyl)thiazol-2-yl)piperidin-4-ylcarbamate (78.7 mg, 0.180 mmol) in CH₂Cl₂ (2.00 mL) was treated at 0° C. with TFA (1.00 mL). The mixture was stirred at 0° C. for 30 min and room temperature for 10 min. The solvents were removed under vacuo. The crude product was filtered through a short cartridge column to remove TFA salt to give 43.4 mg (72%) product as a white solid. Small portion of sample was re-purified by HPLC under acidic condition to give the product as a TFA salt: ¹H NMR (400 MHz, DMSO-d₆) δ ppm 7.93 (br. s., 2H), 7.72-7.85 (m, 2H), 7.50-7.65 (m, 2H), 7.37 (s, 1H), 4.00 (d, 2H), 3.25-3.35 (m, 1H), 3.14 (td, J=12.8, 2.6 Hz, 2H), 1.99 (dd, J=12.6, 3.0 Hz, 2H), 1.57 (qd, J=12.4, 4.4 Hz, 2H); ¹⁹F NMR (376 MHz, DMSO-d₆) δ ppm −73.54 (s); LCMS RT=4.657 min, m/z 338.0 [M+H⁺]; HRMS (ESI) m/z calcd for C₁₄H₁₇⁷⁹BrN₃S [M+H⁺] 338.0327.

### Example 112

### 2-(4-(1H-Tetrazol-5-yl)Piperidin-1-yl)-4-(4-Bromophenyl)Thiazole (XJB02-077)

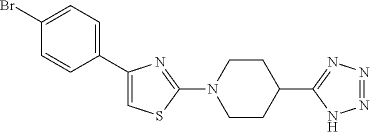 [SMILES: Brc1ccc(-c2csc(N3CCC(c4nnn[nH]4)CC3)n2)cc1]

A mixture of 1-(4-(4-bromophenyl)thiazol-2-yl)piperidine-4-carbonitrile (59.5 mg, 0.171 mmol), sodium azide (33.3 mg, 0.513 mmol) and zinc bromide (57.7 mg, 0.256 mmol) in water (1.00 mL) and 1,4-dioxane (0.58 mL). The pH of the solution was adjusted to about 7 with 1 N NaOH (˜6 drops). The mixture was heated in oil bath at 120° C. for 60 hours. Another aqoite of reagents was added and the mixture was re-heated at 120° C. for 60 hours. The mixture was cooled to room temperature, diluted with EtOAc and washed with H₂O. The organic layer was separated, dried and concentrated in vacuo. The crude product was purified by preparative HPLC under basic condition to give 15.0 mg (22%) product: ¹H NMR (400 MHz, DMSO-d₆) δ ppm 7.76-7.87 (m, 2H), 7.53-7.62 (m, 2H), 7.33 (s, 1H), 3.97 (dt, J=13.2, 3.7 Hz, 2H), 3.20-3.30 (m, 2H), 3.14 (tt, J=11.1, 3.6 Hz, 1H), 2.04 (dd, J=13.3, 3.1 Hz, 2H), 1.71-1.87 (m, 2H); LCMS RT=5.616 min, m/z 391.0 [M+H⁺]; HRMS (ESI) m/z calcd for C₁₅H₁₆⁷⁹BrN₆S [M+H⁺] 391.0341.

### Example 113

### N-(1-(4-(4-Bromophenyl)Thiazol-2-yl)Piperidin-4-yl)Acetamide (XJB02-080)

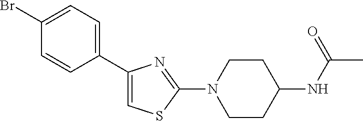 [SMILES: CC(=O)NC1CCN(c2nc(-c3ccc(Br)cc3)cs2)CC1]

The compound was prepared according to the general protocol C: ¹H NMR (400 MHz, CHLOROFORM-d) δ ppm 7.59-7.83 (m, 2H), 7.45-7.60 (m, 2H), 6.77 (s, 1H), 5.36 (d, J=7.4 Hz, 1H), 3.89-4.19 (m, 3H), 3.18 (ddd, J=13.4, 12.0, 2.8 Hz, 2H), 2.03-2.13 (m, 2H), 2.00 (s, 3H), 1.46-1.57 (m, 2H); LCMS RT=5.476 min, m/z 380.0 [M+H⁺]; HRMS (ESI) m/z calcd for C₁₆H₁₉⁷⁹BrN₃OS [M+H⁺] 380.0432.

### Example 114

### (4-(4-(4-Bromophenyl)Thiazol-2-yl)Piperazin-1-yl)(Furan-2-yl)Methanone (XJB02-081)

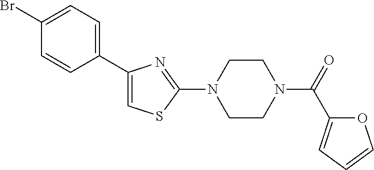 [SMILES: O=C(c1ccco1)N1CCN(c2nc(-c3ccc(Br)cc3)cs2)CC1]

The compound was prepared according to the general protocol C: ¹H NMR (400 MHz, CHLOROFORM-d) δ ppm 7.66-7.77 (m, 2H), 7.45-7.55 (m, 3H), 7.09 (dd, J=3.5, 1.0 Hz, 1H), 6.83 (s, 1H), 6.53 (dd, J=3.5, 1.8 Hz, 1H), 3.99 (br. s., 4H), 3.66 (dd, J=6.2, 4.4 Hz, 4H); LCMS RT=6.560 min, m/z 418.0 [M+H⁺]; HRMS (ESI) m/z calcd for C₁₈H₁₇⁷⁹BrN₃O₂8 [M+H⁺] 418.0225.

### Example 115

### (4-(4-(4-Bromophenyl)Thiazol-2-yl)Piperazin-1-yl)(Phenyl)Methanone (XJB02-082)

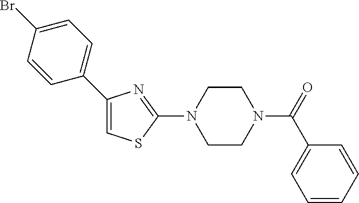 [SMILES: O=C(c1ccccc1)N1CCN(c2nc(-c3ccc(Br)cc3)cs2)CC1]

The compound was prepared according to the general protocol C: ¹H NMR (400 MHz, CHLOROFORM-d) δ ppm 7.60-7.77 (m, 2H), 7.38-7.56 (m, 7H), 6.83 (s, 1H), 3.77-4.12 (m, 2H), 3.61 (br. s., 6H); LCMS RT=6.836 min, m/z 428.0 [M+H⁺]; HRMS (ESI) m/z calcd for C₂₀H₁₉⁷⁹BrN₃OS [M+H⁺] 428.0432.

### Example 116

### 4-(4-Bromophenyl)-2-(4-(Phenylsulfonyl)Piperazin-1-yl)Thiazole (XJB02-083)

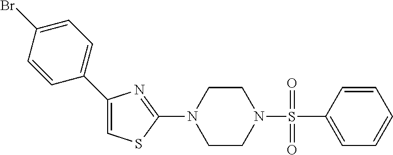 [SMILES: O=S(=O)(c1ccccc1)N1CCN(c2nc(-c3ccc(Br)cc3)cs2)CC1]

The compound was prepared according to the general protocol C: ¹H NMR (400 MHz, CHLOROFORM-d) δ ppm 7.73-7.88 (m, 2H), 7.59-7.71 (m, 3H), 7.53-7.60 (m, 2H), 7.43-7.52 (m, 2H), 6.77 (s, 1H), 3.55-3.81 (m, 4H), 3.09-3.28 (m, 4H); LCMS RT=7.226 min, m/z 464.0 [M+H⁺]; HRMS (ESI) m/z calcd for C₁₉H₁₉⁷⁹BrN₃O₂S₂ [M+H⁺] 464.0102.

### Example 117

### 4-(4-Bromophenyl)-2-(4-(Methylsulfonyl)Piperazin-1-yl)Thiazole (XJB02-084)

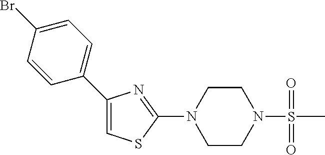 [SMILES: CS(=O)(=O)N1CCN(c2nc(-c3ccc(Br)cc3)cs2)CC1]

The compound was prepared according to the general protocol C: ¹H NMR (400 MHz, CHLOROFORM-d) δ ppm 7.60-7.96 (m, 2H), 7.44-7.60 (m, 2H), 6.84 (s, 1H), 3.56-3.88 (m, 4H), 3.29-3.50 (m, 4H), 2.84 (s, 3H); LCMS RT=6.368 min, m/z 402.0 [M+H⁺]; HRMS (ESI) m/z calcd for C₁₄H₁₇⁷⁹BrN₃O₂S₂ [M+H⁺] 401.9946.

### Example 118

### 4-(4-(4-Bromophenyl)Thiazol-2-yl)Piperazine-1-Carboxamide (XJB02-085)

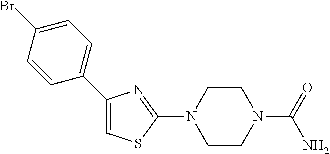 [SMILES: NC(=O)N1CCN(c2nc(-c3ccc(Br)cc3)cs2)CC1]

A solution of 4-(4-bromophenyl)-2-(piperazin-1-yl)thiazole (40.0 mg, 0.123 mmol) in H₂O (2.00 mL) was treated at room temperature with KOCN (20.0 mg, 0.247 mmol). The reaction mixture was stirred at room temperature for overnight. The mixture was extracted with EtOAc. The organic layer was separated, dried, and concentrated in vacuo. The crude mixture was purified by preparative HPLC under basic condition to give 14.2 mg product (31%) product: ¹H NMR (400 MHz, DMSO-d₆) δ ppm 7.82 (d, J=8.4 Hz, 2H), 7.57 (d, J=8.4 Hz, 2H), 7.37 (s, 1H), 6.11 (br. s., 2H), 3.37-3.57 (m, 8H); LCMS RT=5.329 min, m/z 367.0 [M+H⁺]; HRMS (ESI) m/z calcd for C₁₄H₁₆⁷⁹BrN₄OS [M+H⁺] 367.0228.

### Example 119

### 1-(5-P-Tolylthiazol-2-yl)Piperidine-4-Carboxamide (XJB03-049)

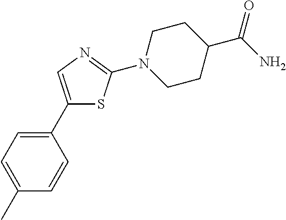 [SMILES: Cc1ccc(-c2cnc(N3CCC(C(N)=O)CC3)s2)cc1]

The compound was prepared according to the general protocol A as a TFA salt: ¹H NMR (400 MHz, DMSO-d₆) δ ppm 7.58 (s, 1H), 7.34-7.45 (m, 2H), 7.32 (br. s., 1H), 7.18 (d, J=8.0 Hz, 2H), 6.84 (br. s., 1H), 3.91 (dt, J=12.9, 3.2 Hz, 2H), 3.12 (td, J=12.6, 2.8 Hz, 2H), 2.38 (tt, J=11.4, 3.8 Hz, 1H), 2.29 (s, 3H), 1.82 (dd, J=13.2, 2.8 Hz, 2H), 1.48-1.72 (m, 3H); ¹⁹F NMR (376 MHz, DMSO-d₆) δ ppm −74.86 (s); LCMS RT=3.912 min, m/z 302.1 [M+H⁺]; HRMS (ESI) m/z calcd for C₁₆H₂₀NOS [M+H⁺] 302.1327.

### Example 120

### 1-(5-(2,3-Dihydrobenzo[B][1,4]Dioxin-6-yl)Thiazol-2-yl)Piperidine-4-Carboxamide (XJB03-050)

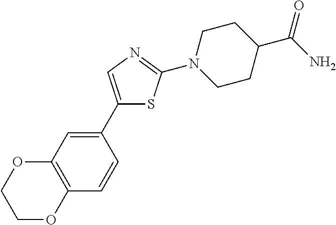 [SMILES: NC(=O)C1CCN(c2ncc(-c3ccc4c(c3)OCCO4)s2)CC1]

The compound was prepared according to the general protocol A: ¹H NMR (400 MHz, DMSO-d₆) δ ppm 7.39-7.48 (m, 1H), 7.31 (br. s., 1H), 6.95 (d, J=2.0 Hz, 1H), 6.87-6.93 (m, 1H), 6.74-6.86 (m, 2H), 4.24 (s, 4H), 3.78-3.95 (m, 2H), 3.03 (td, J=12.5, 2.6 Hz, 2H), 2.35 (tt, J=11.5, 4.1 Hz, 1H), 1.79 (dd, J=13.5, 2.3 Hz, 2H), 1.47-1.69 (m, 2H); LCMS RT=3.589 min, m/z 346.1 [M+H⁺]; HRMS (ESI) m/z calcd for C₁₇H₂₀N₃O₃S [M+H⁺] 346.1225.

### Example 121

### 1-(5-(3-Fluorophenyl)Thiazol-2-yl)Piperidine-4-Carboxamide (XJB03-051)

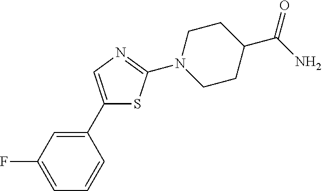 [SMILES: NC(=O)C1CCN(c2ncc(-c3cccc(F)c3)s2)CC1]

The compound was prepared according to the general protocol A: ¹H NMR (400 MHz, DMSO-d₆) δ ppm 7.69 (s, 1H), 7.33-7.42 (m, 2H), 7.30-7.33 (m, 1H), 7.25 (ddd, J=7.7, 1.7, 1.0 Hz, 1H), 6.97-7.06 (m, 1H), 6.83 (br. s., 1H), 3.93 (dt, J=12.8, 3.3 Hz, 2H), 3.09 (td, J=12.6, 2.9 Hz, 2H), 2.37 (tt, J=11.4, 3.7 Hz, 1H), 1.81 (dd, J=13.4, 3.0 Hz, 2H), 1.50-1.67 (m, 2H); LCMS RT=3.952 min, m/z 306.1 [M+H⁺]; HRMS (ESI) m/z calcd for C₁₅H₁₇FN₃OS [M+H⁺] 306.1076.

### Example 122

### 1-(5-(4-Chlorophenyl)Thiazol-2-yl)Piperidine-4-Carboxamide (XJB03-053)

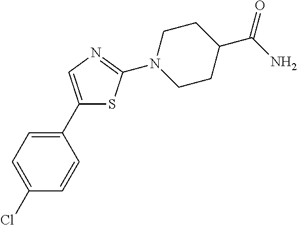 [SMILES: NC(=O)C1CCN(c2ncc(-c3ccc(Cl)cc3)s2)CC1]

The compound was prepared according to the general protocol A as a TFA salt: ¹H NMR (400 MHz, DMSO-d₆) δ ppm 7.66 (s, 1H), 7.46-7.53 (m, 2H), 7.37-7.44 (m, 2H), 7.32 (br. s., 1H), 6.83 (br. s., 1H), 3.92 (ddd, J=13.0, 3.6, 3.4 Hz, 2H), 3.11 (td, J=12.6, 3.0 Hz, 2H), 2.37 (tt, J=11.4, 3.7 Hz, 1H), 1.81 (dd, J=12.9, 2.9 Hz, 2H), 1.53-1.72 (m, 2H); ¹⁹F NMR (376 MHz, DMSO-d₆) δ ppm −74.87 (s); LCMS RT=4.329 min, m/z 322.0 [M+H⁺]; HRMS (ESI) m/z calcd for C₁₅H₁₇³⁵ClN₃OS [M+H⁺] 322.0781.

### Example 123

### 1-(5-(3-Isopropoxyphenyl)Thiazol-2-yl)Piperidine-4-Carboxamide (XJB03-054)

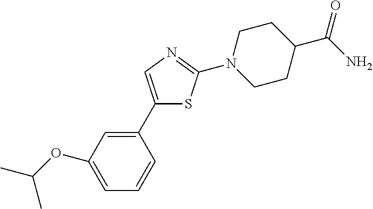 [SMILES: CC(C)Oc1cccc(-c2cnc(N3CCC(C(N)=O)CC3)s2)c1]

The compound was prepared according to the general protocol A as a TFA salt: ¹H NMR (400 MHz, DMSO-d₆) δ ppm 7.65 (s, 1H), 7.32 (br. s., 1H), 7.24 (t, J=8.0 Hz, 1H), 6.93-7.03 (m, 2H), 6.83 (br. s., 1H), 6.76-6.81 (m, 1H), 4.55-4.75 (m, 1H), 3.91 (dt, J=12.8, 3.4 Hz, 2H), 3.11 (td, J=12.5, 2.9 Hz, 2H), 2.37 (tt, J=11.4, 3.7 Hz, 1H), 1.81 (dd, J=13.3, 2.9 Hz, 2H), 1.52-1.68 (m, 2H), 1.26 (d, J=6.1 Hz, 6H); ¹⁹F NMR (376 MHz, DMSO-d₆) δ ppm −74.91 (s); LCMS RT=4.300 min, m/z 346.1 [M+H⁺]; HRMS (ESI) m/z calcd for C₁₈H₂₄N₃O₂S [M+H⁺] 346.1589.

### Example 124

### 1-(5-(3,4-Dihydro-2H-Benzo[B][1,4] Dioxepin-7-yl)Thiazol-2-yl)Piperidine-4-Carboxamide (XJB03-055)

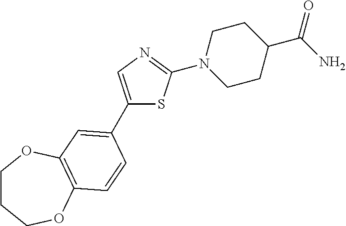 [SMILES: NC(=O)C1CCN(c2ncc(-c3ccc4c(c3)OCCCO4)s2)CC1]

The compound was prepared according to the general protocol A: ¹H NMR (400 MHz, DMSO-d₆) δ ppm 7.47 (s, 1H), 7.31 (br. s., 1H), 7.07 (d, J=2.3 Hz, 1H), 6.97-7.04 (m, 1H), 6.89-6.96 (m, 1H), 6.82 (br. s., 1H), 4.12 (dt, J=9.3, 5.5 Hz, 4H), 3.89 (dt, J=12.8, 3.3 Hz, 2H), 3.04 (td, J=12.5, 2.9 Hz, 2H), 2.36 (tt, J=11.5, 3.7 Hz, 1H), 2.02-2.15 (m, 2H), 1.79 (dd, J=13.2, 2.8 Hz, 2H), 1.50-1.68 (m, 2H); LCMS RT=3.735 min, m/z 360.1 [M+H⁺]; HRMS (ESI) m/z calcd for C₁₈H₂₂N₃O₃S [M+H⁺] 360.1382.

### Example 125

### 1-(5-(Benzo[D][1,3]Dioxol-5-yl)Thiazol-2-yl)Piperidine-4-Carboxamide (XJB03-056)

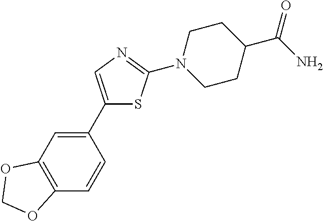 [SMILES: NC(=O)C1CCN(c2ncc(-c3ccc4c(c3)OCO4)s2)CC1]

The compound was prepared according to the general protocol A: ¹H NMR (400 MHz, DMSO-d₆) δ ppm 7.46 (s, 1H), 7.31 (br. s., 1H), 7.13 (d, J=1.8 Hz, 1H), 6.84-6.94 (m, 2H), 6.82 (br. s., 1H), 6.02 (s, 2H), 3.89 (dt, J=12.8, 3.3 Hz, 2H), 3.04 (td, J=12.6, 2.9 Hz, 2H), 2.35 (tt, J=11.4, 3.7 Hz, 1H), 1.79 (dd, J=13.1, 2.9 Hz, 2H), 1.50-1.69 (m, 2H); LCMS RT=3.579 min, m/z 332.1 [M+H⁺]; HRMS (ESI) m/z calcd for C₁₆H₁₈N₃O₃S [M+H⁺] 332.1069.

### Example 126

### 1-(4-P-Tolylpyrimidin-2-yl)Piperidine-4-Carboxamide (XJB03-059)

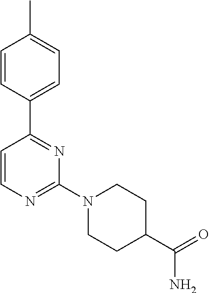 [SMILES: Cc1ccc(-c2ccnc(N3CCC(C(N)=O)CC3)n2)cc1]

The compound was prepared according to the general protocol A as a TFA salt: ¹H NMR (400 MHz, DMSO-d₆) δ ppm 8.39 (d, J=5.1 Hz, 1H), 7.97-8.10 (m, 2H), 7.32 (dd, J=8.5, 0.7 Hz, 2H), 7.29 (br. s., 1H), 7.16 (d, J=5.3 Hz, 1H), 6.78 (br. s., 1H), 4.70-4.82 (m, 2H), 2.88-3.07 (m, 2H), 2.39-2.46 (m, 1H), 2.37 (s, 3H), 1.80 (dd, J=13.4, 3.0 Hz, 2H), 1.50 (qd, J=12.4, 4.3 Hz, 2H); ¹⁹F NMR (376 MHz, DMSO-d₆) δ ppm −74.83 (s); LCMS RT=3.947 min, m/z 297.1 [M+H⁺]; HRMS (ESI) m/z calcd for C₁₇H₂₁N₄O [M+H⁺] 297.1715.

### Example 127

### 1-(4-(3-Isopropoxyphenyl)Pyrimidin-2-yl)Piperidine-4-Carboxamide (XJB03-060)

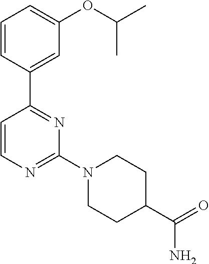 [SMILES: CC(C)Oc1cccc(-c2ccnc(N3CCC(C(N)=O)CC3)n2)c1]

The compound was prepared according to the general protocol A as a TFA salt: ¹H NMR (400 MHz, DMSO-d₆) δ ppm 8.41 (d, J=5.1 Hz, 1H), 7.66 (ddd, J=8.1, 1.3, 1.0 Hz, 1H), 7.57-7.63 (m, 1H), 7.41 (t, J=7.9 Hz, 1H), 7.29 (br. s., 1H), 7.19 (d, J=5.3 Hz, 1H), 7.03-7.13 (m, 1H), 6.78 (br. s., 1H), 4.55-4.84 (m, 3H), 2.88-3.10 (m, 2H), 2.41 (tt, J=11.5, 3.8 Hz, 1H), 1.80 (dd, J=12.7, 2.9 Hz, 2H), 1.50 (qd, J=12.4, 3.9 Hz, 2H), 1.30 (d, J=6.1 Hz, 6H); ¹⁹F NMR (376 MHz, DMSO-d₆) δ ppm −74.96 (s); LCMS RT=4.454 min, m/z 341.2 [M+H⁺]; HRMS (ESI) m/z calcd for C₁₉H₂₅N₄O₂ [M+H⁺] 341.1978.

### Example 128

### 1-(4-(3,4-Dihydro-2H-Benzo[B][1,4]Dioxepin-7-yl)Pyrimidin-2-yl)Piperidine-4-Carboxamide (XJB03-061)

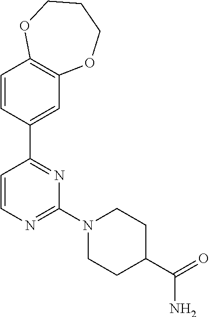 [SMILES: NC(=O)C1CCN(c2nccc(-c3ccc4c(c3)OCCCO4)n2)CC1]

The compound was prepared according to the general protocol A: ¹H NMR (400 MHz, DMSO-d₆) δ ppm 8.36 (d, J=5.1 Hz, 1H), 7.62-7.80 (m, 2H), 7.29 (br. s., 1H), 7.01-7.14 (m, 2H), 6.78 (br. s., 1H), 4.63-4.84 (m, 2H), 4.10-4.26 (m, 4H), 2.93 (td, J=12.7, 2.5 Hz, 2H), 2.40 (tt, J=11.5, 3.7 Hz, 1H), 2.14 (quin, J=5.6 Hz, 2H), 1.79 (dd, J=12.9, 2.7 Hz, 2H), 1.49 (qd, J=12.4, 4.3 Hz, 2H); LCMS RT=3.836 min, m/z 355.2 [M+H⁺]; HRMS (ESI) m/z calcd for C₁₉H₂₃N₄O₃ [M+H⁺] 355.1770.

### Example 129

### 1-(4-(2,3-Dihydrobenzo[B][1,4]Dioxin-6-yl)Pyrimidin-2-yl)Piperidine-4-Carboxamide (XJB03-062)

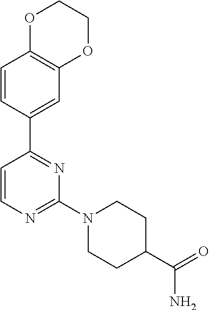 [SMILES: NC(=O)C1CCN(c2nccc(-c3ccc4c(c3)OCCO4)n2)CC1]

The compound was prepared according to the general protocol A: ¹H NMR (400 MHz, DMSO-d₆) δ ppm 8.34 (d, J=5.1 Hz, 1H), 7.47-7.77 (m, 2H), 7.29 (br. s., 1H), 7.09 (d, J=5.3 Hz, 1H), 6.90-7.05 (m, 1H), 6.77 (br. s., 1H), 4.60-4.95 (m, 2H), 4.09-4.42 (m, 4H), 2.93 (td, J=12.7, 2.5 Hz, 2H), 2.39 (tt, J=11.5, 3.7 Hz, 1H), 1.78 (dd, J=13.1, 2.7 Hz, 2H), 1.49 (qd, J=12.4, 4.2 Hz, 2H); LCMS RT=3.621 min, m/z 341.1 [M+H⁺]; HRMS (ESI) m/z calcd for C₁₈H₂₁N₄O₃ [M+H⁺] 341.1614.

### Example 130

### 1-(5-(3,4-Dihydro-2H-Benzo[B][1,4]Dioxepin-7-yl)-1,3,4-Thiadiazol-2-yl)Piperidine-4-Carboxamide (XJB03-063)

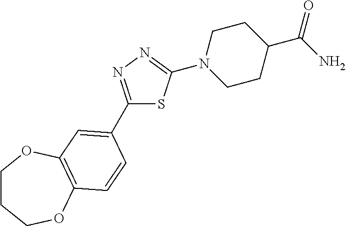 [SMILES: NC(=O)C1CCN(c2nnc(-c3ccc4c(c3)OCCCO4)s2)CC1]

The compound was prepared according to the general protocol A as a TFA salt: ¹H NMR (400 MHz, DMSO-d₆) δ ppm 7.23-7.52 (m, 3H), 6.95-7.11 (m, 1H), 6.84 (br. s., 1H), 4.19 (t, J=5.6 Hz, 4H), 3.88 (dt, J=12.8, 3.5 Hz, 2H), 3.19 (td, J=12.4, 2.9 Hz, 2H), 2.35 (tt, J=11.4, 4.0 Hz, 1H), 2.14 (dt, J=11.2, 5.6 Hz, 2H), 1.82 (dd, J=13.5, 2.9 Hz, 2H), 1.65 (qd, J=12.4, 3.9 Hz, 2H); ¹⁹F NMR (376 MHz, DMSO-d₆) δ ppm −74.79 (s); LCMS RT=4.210 min, m/z 361.1 [M+H⁺]; HRMS (ESI) m/z calcd for C₁₇H₂₁N₄O₃S [M+H⁺] 361.1334.

### Example 131

### 1-(5-(4-Bromophenyl)-1,3,4-Thiadiazol-2-yl)Piperidine-4-Carboxamide (XJB03-065-B)

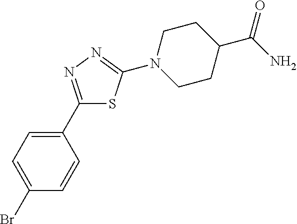 [SMILES: NC(=O)C1CCN(c2nnc(-c3ccc(Br)cc3)s2)CC1]

The compound was prepared according to the general protocol A: ¹H NMR (400 MHz, DMSO-d₆) δ ppm 7.43-7.95 (m, 4H), 7.33 (br. s., 1H), 6.84 (br. s., 1H), 3.91 (dt, J=12.9, 3.3 Hz, 2H), 3.22 (td, J=12.5, 2.9 Hz, 2H), 2.39 (tt, J=11.3, 3.6 Hz, 1H), 1.83 (dd, J=13.4, 3.0 Hz, 2H), 1.52-1.75 (m, 2H); LCMS RT=4.767 min, m/z 367.0 [M+H⁺]; HRMS (ESI) m/z calcd for C₁₄H₁₆⁷⁹BrN₄OS [M+H⁺] 367.0228.

### Example 132

### 1-(5-(3-(Dimethylamino)Phenyl)-1,3,4-Thiadiazol-2-yl)Piperidine-4-Carboxamide (XJB03-066-B)

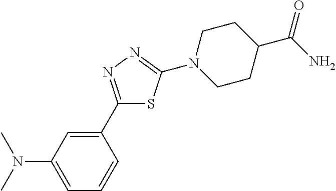 [SMILES: CN(C)c1cccc(-c2nnc(N3CCC(C(N)=O)CC3)s2)c1]

The compound was prepared according to the general protocol A: ¹H NMR (400 MHz, DMSO-d₆) δ ppm 7.32 (br. s., 1H), 7.26 (dd, J=8.2, 7.6 Hz, 1H), 7.05 (dd, J=2.4, 1.7 Hz, 1H), 7.00 (ddd, J=7.5, 1.6, 0.9 Hz, 1H), 6.83 (br. s., 1H), 6.79-6.83 (m, 1H), 3.90 (dt, J=12.9, 3.2 Hz, 2H), 3.19 (td, J=12.6, 2.9 Hz, 2H), 2.94 (s, 6H), 2.38 (tt, J=11.5, 3.7 Hz, 1H), 1.82 (dd, J=13.4, 3.2 Hz, 2H), 1.55-1.72 (m, 2H); LCMS RT=3.411 min, m/z 332.1 [M+H⁺]; HRMS (ESI) m/z calcd for C₁₆H₂₂N₅OS [M+H⁺] 332.1545.

### Example 133

### 1-(5-P-Tolyl-1,3,4-Thiadiazol-2-yl)Piperidine-4-Carboxamide (XJB03-067-B)

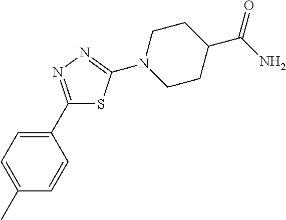 [SMILES: Cc1ccc(-c2nnc(N3CCC(C(N)=O)CC3)s2)cc1]

The compound was prepared according to the general protocol A: ¹H NMR (400 MHz, DMSO-d₆) δ ppm 7.58-7.72 (m, 2H), 7.32 (br. s., 1H), 7.30 (dd, J=8.5, 0.7 Hz, 2H), 6.83 (br. s., 1H), 3.90 (dt, J=13.0, 3.4 Hz, 2H), 3.20 (td, J=12.6, 3.0 Hz, 2H), 2.36-2.43 (m, 1H), 2.35 (s, 3H), 1.82 (dd, J=13.6, 3.4 Hz, 2H), 1.56-1.70 (m, 2H); LCMS RT=4.396 min, m/z 303.1 [M+H⁺]; HRMS (ESI) m/z calcd for C₁₅H₁₉N₄OS [M+H⁺] 303.1280.

### Example 134

### 1-(5-(4-(Methylthio)Phenyl)Thiazol-2-yl)Piperidine-4-Carboxamide (XJB03-068)

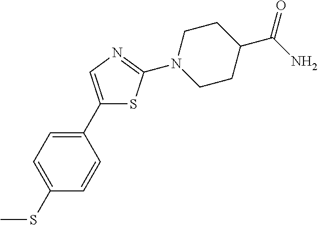 [SMILES: CSc1ccc(-c2cnc(N3CCC(C(N)=O)CC3)s2)cc1]

The compound was prepared according to the general protocol A as a TFA salt: ¹H NMR (400 MHz, DMSO-d₆) δ ppm 7.58 (s, 1H), 7.37-7.45 (m, 2H), 7.32 (br. s., 1H), 7.21-7.29 (m, 2H), 6.83 (br. s., 1H), 3.91 (dt, J=12.9, 3.0 Hz, 2H), 3.09 (td, J=12.5, 2.8 Hz, 2H), 2.48 (s, 3H), 2.37 (tt, J=11.6, 4.0 Hz, 1H), 1.81 (dd, J=13.5, 3.5 Hz, 2H), 1.54-1.67 (m, 2H); ¹⁹F NMR (376 MHz, DMSO-d₆) δ ppm −74.56 (s); LCMS RT=4.072 min, m/z 361.1 [M+H⁺]; HRMS (ESI) m/z calcd for C₁₆H₂₀N₃OS₂ [M+H⁺] 334.1048.

### Example 135

### 2-(4-(1H-Imidazol-2-yl)Piperidin-1-yl)-5-(3,4-Dihydro-2H-Benzo[B][1,4] Dioxepin-7-yl)Thiazole (XJB03-080)

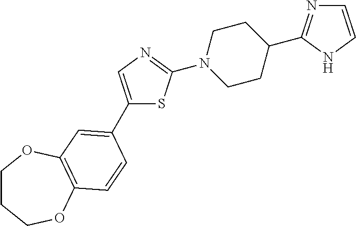 [SMILES: c1c[nH]c(C2CCN(c3ncc(-c4ccc5c(c4)OCCCO5)s3)CC2)n1]

The compound was prepared according to the general protocol A: ¹H NMR (400 MHz, DMSO-d₆) δ ppm 11.74 (br. s., 1H), 7.49 (s, 1H), 7.08 (d, J=2.2 Hz, 1H), 6.99-7.04 (m, 1H), 6.92-6.96 (m, 1H), 6.88 (br. s., 2H), 4.13 (dt, J=9.4, 5.5 Hz, 4H), 3.93 (ddd, J=12.9, 3.3, 3.0 Hz, 2H), 3.11-3.24 (m, 2H), 2.95 (tt, J=11.4, 3.6 Hz, 1H), 2.05-2.16 (m, 2H), 1.98 (dd, J=13.5, 3.1 Hz, 2H), 1.64-1.84 (m, 2H); LCMS RT=3.562 min, m/z 383.1 [M+H⁺]; HRMS (ESI) m/z calcd for C₂₀H₂₃N₄O₂S [M+H⁺] 383.1542.

### Example 136

### 3-(2-(4-(1H-Imidazol-2-yl)Piperidin-1-yl)Thiazol-5-yl)-N,N-Dimethylaniline (XJB03-081)

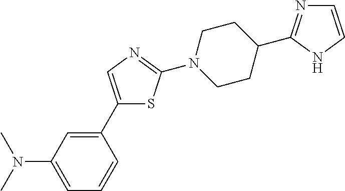 [SMILES: CN(C)c1cccc(-c2cnc(N3CCC(c4ncc[nH]4)CC3)s2)c1]

The compound was prepared according to the general protocol A as a TFA salt: ¹H NMR (400 MHz, DMSO-d₆) δ ppm 7.47-7.70 (m, 3H), 7.15-7.29 (m, 1H), 6.79-6.90 (m, 2H), 6.70 (dd, J=8.2, 1.6 Hz, 1H), 3.94-4.17 (m, 2H), 3.36 (tt, J=12.0, 3.6 Hz, 1H), 3.24 (td, J=12.8, 2.6 Hz, 2H), 2.95 (s, 6H), 2.09 (dd, J=13.0, 2.4 Hz, 2H), 1.88 (qd, J=12.5, 4.2 Hz, 2H); ¹⁹F NMR (376 MHz, DMSO-d₆) δ ppm −74.72 (s); LCMS RT=3.050 min, m/z 354.2 [M+H⁺]; HRMS (ESI) m/z calcd for C₁₉H₂₄N₅S [M+H⁺] 354.1752.

### Example 137

### 1-(5-(3-(Pyrrolidin-1-yl)Phenyl)Thiazol-2-yl)Piperidine-4-Carboxamide (XJB03-082)

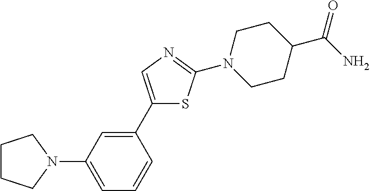 [SMILES: NC(=O)C1CCN(c2ncc(-c3cccc(N4CCCC4)c3)s2)CC1]

The compound was prepared according to the general protocol A: ¹H NMR (400 MHz, DMSO-d₆) δ ppm 7.51 (s, 1H), 7.31 (br. s., 1H), 7.00-7.17 (m, 1H), 6.82 (br. s., 1H), 6.69 (ddd, J=7.6, 1.6, 0.8 Hz, 1H), 6.54 (t, J=2.0 Hz, 1H), 6.34-6.45 (m, 1H), 3.92 (dt, J=12.7, 3.1 Hz, 2H), 3.18-3.28 (m, 4H), 3.05 (td, J=12.5, 2.9 Hz, 2H), 2.36 (tt, J=11.5, 3.8 Hz, 1H), 1.95 (dt, J=6.5, 3.3 Hz, 4H), 1.79 (dd, J=13.6, 3.2 Hz, 2H), 1.50-1.68 (m, 2H); LCMS RT=4.159 min, m/z 357.1 [M+H⁺]; HRMS (ESI) m/z calcd for C₁₉H₂₅N₄OS [M+H⁺] 357.1749.

### Example 138

### 1-(5-(3-(Piperidin-1-yl)Phenyl)Thiazol-2-yl)Piperidine-4-Carboxamide (XJB03-083)

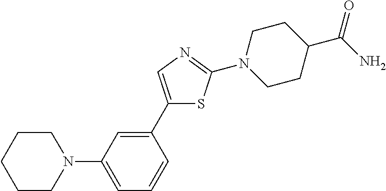 [SMILES: NC(=O)C1CCN(c2ncc(-c3cccc(N4CCCCC4)c3)s2)CC1]

The compound was prepared according to the general protocol A: ¹H NMR (400 MHz, DMSO-d₆) δ ppm 7.55 (s, 1H), 7.31 (br. s., 1H), 7.16 (t, J=7.9 Hz, 1H), 6.95 (s, 1H), 6.72-6.88 (m, 3H), 3.92 (dt, J=12.9, 3.2 Hz, 2H), 3.11-3.21 (m, 4H), 3.05 (td, J=12.6, 2.8 Hz, 2H), 2.36 (tt, J=11.5, 3.8 Hz, 1H), 1.80 (dd, J=13.6, 3.2 Hz, 2H), 1.57-1.68 (m, 6H), 1.49-1.57 (m, 2H); LCMS RT=3.104 min, m/z 371.2 [M+H⁺]; HRMS (ESI) m/z calcd for C₂₀H₂₇N₄OS [M+H⁺] 371.1906.

### Example 139

### 1-(5-(3-Morpholinophenyl)Thiazol-2-yl)Piperidine-4-Carboxamide (XJB03-084)

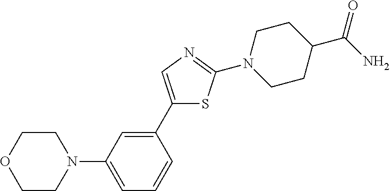 [SMILES: NC(=O)C1CCN(c2ncc(-c3cccc(N4CCOCC4)c3)s2)CC1]

The compound was prepared according to the general protocol A: ¹H NMR (400 MHz, DMSO-d₆) δ ppm 7.57 (s, 1H), 7.31 (br. s., 1H), 7.14-7.24 (m, 1H), 6.96-7.01 (m, 1H), 6.89 (ddd, J=7.6, 1.5, 0.7 Hz, 1H), 6.78-6.86 (m, 2H), 3.87-3.98 (m, 2H), 3.68-3.83 (m, 4H), 3.11-3.19 (m, 4H), 3.06 (td, J=12.5, 2.9 Hz, 2H), 2.36 (tt, J=11.4, 3.6 Hz, 1H), 1.80 (dd, J=13.4, 3.0 Hz, 2H), 1.62 (qd, J=12.4, 4.3 Hz, 2H); LCMS RT=3.580 min, m/z 373.1 [M+H⁺]; HRMS (ESI) m/z calcd for C₁₉H₂₅N₄O₂S [M+H⁺] 373.1698.

### Example 140

### 1-(1-(5-(3,4-Dihydro-2H-Benzo[B][1,4]Dioxepin-7-yl)Thiazol-2-yl)Piperidin-4-yl)Ethanone (XJB03-093-B)

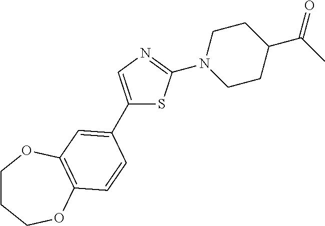 [SMILES: CC(=O)C1CCN(c2ncc(-c3ccc4c(c3)OCCCO4)s2)CC1]

The compound was prepared according to the general protocol A: ¹H NMR (400 MHz, DMSO-d₆) δ ppm 7.47 (s, 1H), 7.07 (d, J=2.0 Hz, 1H), 6.98-7.03 (m, 1H), 6.91-6.97 (m, 1H), 4.12 (dt, J=9.0, 5.5 Hz, 4H), 3.89 (ddd, J=12.9, 3.5, 3.4 Hz, 2H), 3.04-3.13 (m, 2H), 2.64 (tt, J=11.2, 3.5 Hz, 1H), 2.15 (s, 3H), 2.06-2.13 (m, 2H), 1.92 (dd, J=13.6, 3.0 Hz, 2H), 1.43-1.56 (m, 2H); LCMS RT=4.410 min, m/z 359.1 [M+H⁺]; HRMS (ESI) m/z calcd for C₁₉H₂₃N₂O₃S [M+H⁺] 359.1429.

### Example 141

### 1-(1-(5-(3-(Dimethylamino)Phenyl)Thiazol-2-yl)Piperidin-4-yl)Ethanone (XJB03-094-B)

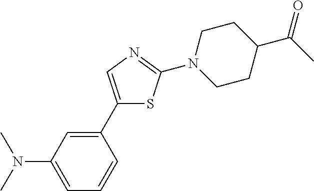 [SMILES: CC(=O)C1CCN(c2ncc(-c3cccc(N(C)C)c3)s2)CC1]

The compound was prepared according to the general protocol A: ¹H NMR (400 MHz, DMSO-d₆) δ ppm 7.53 (s, 1H), 7.06-7.22 (m, 1H), 6.70-6.79 (m, 2H), 6.56-6.63 (m, 1H), 3.91 (dt, J=12.9, 3.3 Hz, 2H), 3.09 (td, J=12.5, 2.9 Hz, 2H), 2.91 (s, 6H), 2.65 (tt, J=11.3, 3.6 Hz, 1H), 2.15 (s, 3H), 1.86-1.99 (m, 2H), 1.43-1.59 (m, 2H); LCMS RT=3.691 min, m/z 330.1 [M+H⁺]; HRMS (ESI) m/z calcd for C₁₈H₂₄N₃OS [M+H⁺] 330.1640.

### Example 142

### 1-(6-(3,4-Dihydro-2H-Benzo[B][1,4]Dioxepin-7-yl)Pyrimidin-4-yl)Piperidine-4-Carboxamide (XJB03-095)

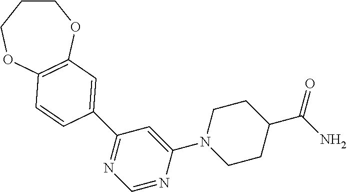 [SMILES: NC(=O)C1CCN(c2cc(-c3ccc4c(c3)OCCCO4)ncn2)CC1]

The compound was prepared according to the general protocol A: ¹H NMR (400 MHz, DMSO-d₆) δ ppm 8.50 (d, J=1.0 Hz, 1H), 7.72-7.85 (m, 2H), 7.29 (br. s., 1H), 7.25 (d, J=1.2 Hz, 1H), 7.03 (d, J=8.2 Hz, 1H), 6.79 (br. s., 1H), 4.40-4.73 (m, 2H), 4.06-4.28 (m, 4H), 2.83-3.05 (m, 2H), 2.42 (tt, J=11.4, 4.0 Hz, 1H), 2.14 (quin, J=5.5 Hz, 2H), 1.78 (dd, J=13.4, 2.8 Hz, 2H), 1.38-1.58 (m, 2H); LCMS RT=3.241 min, m/z 355.2 [M+H⁺]; HRMS (ESI) m/z calcd for C₁₉H₂₃N₄O₃ [M+H⁺] 355.1770.

### Example 143

### 1-(6-(3-(Dimethylamino)Phenyl)Pyrimidin-4-yl)Piperidine-4-Carboxamide (XJB03-096)

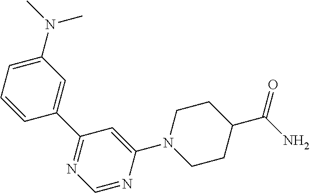 [SMILES: CN(C)c1cccc(-c2cc(N3CCC(C(N)=O)CC3)ncn2)c1]

The compound was prepared according to the general protocol A: ¹H NMR (400 MHz, DMSO-d₆) δ ppm 8.54 (d, J=1.0 Hz, 1H), 7.46 (dd, J=2.3, 1.8 Hz, 1H), 7.41 (ddd, J=7.8, 1.2, 1.0 Hz, 1H), 7.19-7.34 (m, 3H), 6.82-6.90 (m, 1H), 6.79 (br. s., 1H), 4.53 (d, J=14.3 Hz, 2H), 2.88-3.04 (m, 8H), 2.43 (tt, J=11.5, 4.0 Hz, 1H), 1.79 (dd, J=13.5, 2.9 Hz, 2H), 1.51 (qd, J=12.3, 4.0 Hz, 2H); LCMS RT=3.241 min, m/z 355.2 [M+H⁺]; HRMS (ESI) m/z calcd for C₁₈H₂₄N₅O [M+H⁺] 326.1981.

### Example 144

### 1-(4-(3,4-Dihydro-2H-Benzo[B][1,4]Dioxepin-7-yl)-1,3,5-Triazin-2-yl)Piperidine-4-Carboxamide (XJB04-002-B)

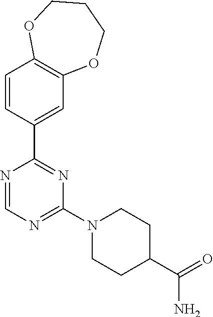 [SMILES: NC(=O)C1CCN(c2ncnc(-c3ccc4c(c3)OCCCO4)n2)CC1]

THE COMPOUND WAS PREPARED ACCORDING TO THE GENERAL PROTOCOL A: 1H NMR (400 MHz, DMSO-D6) □ PPM 8.62 (s, 1H), 7.96 (DD, J=8.4, 2.2 Hz, 1H), 7.92 (D, J=1.8 Hz, 1H), 7.31 (BR. s., 1H), 7.07 (D, J=8.4 Hz, 1H), 6.81 (BR. s., 1H), 4.82 (D, J=13.3 Hz, 1H), 4.67 (D, J=13.3 Hz, 1H), 4.21 (DT, J=13.4, 5.6 Hz, 4H), 2.97-3.14 (M, 2H), 2.43 (TT, J=11.4, 4.1 Hz, 1H), 2.15 (QUIN, J=5.6 Hz, 2H), 1.84 (T, J=14.0 Hz, 2H), 1.41-1.60 (M, 2H); LCMS RT=3.988 MIN, M/Z 356.2 [M+H+]; HRMS (ESI) M/Z CALCD FOR C18H22N5O3 [M+H+] 356.1723.

### Example 145

### 1-(4-(3-(Dimethylamino)Phenyl)-1,3,5-Triazin-2-yl)Piperidine-4-Carboxamide (XJB04-003-B)

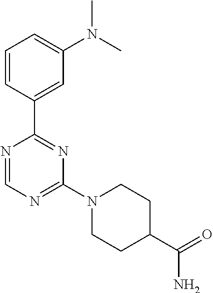 [SMILES: CN(C)c1cccc(-c2ncnc(N3CCC(C(N)=O)CC3)n2)c1]

The compound was prepared according to the general protocol A: ¹H NMR (400 MHz, DMSO-d₆) δ ppm 8.65 (s, 1H), 7.73 (dd, J=2.6, 1.5 Hz, 1H), 7.68 (dt, J=7.6, 1.2 Hz, 1H), 7.19-7.39 (m, 2H), 6.87-7.04 (m, 1H), 6.81 (br. s., 1H), 4.83 (d, J=13.9 Hz, 1H), 4.68 (d, J=12.9 Hz, 1H), 3.00-3.17 (m, 2H), 2.96 (s, 6H), 2.38-2.48 (m, 1H), 1.84 (t, J=11.9 Hz, 2H), 1.42-1.60 (m, 2H); LCMS RT=3.281 min, m/z 327.2 [M+H⁺]; HRMS (ESI) m/z calcd for C₁₇H₂₃N₆O [M+H⁺] 327.1933.

### Example 146

### Ethyl 1-(5-(3,4-Dihydro-2H-Benzo[B][1,4]Dioxepin-7-yl)Thiazol-2-yl)Piperidine-4-Carboxylate (XJB04-004)

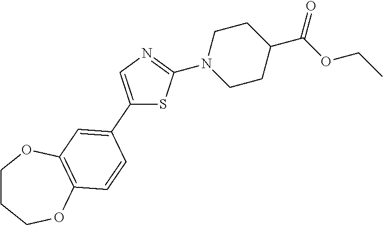 [SMILES: CCOC(=O)C1CCN(c2ncc(-c3ccc4c(c3)OCCCO4)s2)CC1]

The compound was prepared according to the general protocol A: ¹H NMR (400 MHz, DMSO-d₆) δ ppm 7.48 (s, 1H), 7.07 (d, J=2.3 Hz, 1H), 6.99-7.04 (m, 1H), 6.88-6.95 (m, 1H), 4.00-4.18 (m, 6H), 3.84 (dt, J=13.1, 3.5 Hz, 2H), 3.32 (br. s., 2H), 3.13 (ddd, J=12.9, 11.5, 2.9 Hz, 3H), 2.63 (tt, J=11.1, 3.8 Hz, 1H), 2.10 (qd, J=5.5, 5.3 Hz, 2H), 1.85-1.98 (m, 2H), 1.54-1.69 (m, 2H); LCMS RT=5.091 min, m/z 389.1 [M+H⁺]; HRMS (ESI) m/z calcd for C₂₀H₂₅N₂O₄S [M+H⁺] 389.1535.

### Example 147

### 1-(4-(3-(Dimethylamino)Phenyl)-5-Methylpyrimidin-2-yl)Piperidine-4-Carboxamide (XJB04-005)

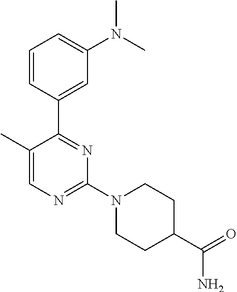 [SMILES: Cc1cnc(N2CCC(C(N)=O)CC2)nc1-c1cccc(N(C)C)c1]

The compound was prepared according to the general protocol A: ¹H NMR (400 MHz, DMSO-d₆) δ ppm 8.25 (d, J=0.8 Hz, 1H), 7.13-7.35 (m, 2H), 6.79-6.90 (m, 3H), 6.75 (br. s., 1H), 4.56-4.73 (m, 2H), 2.92 (s, 6H), 2.87 (td, J=12.6, 2.5 Hz, 2H), 2.30-2.42 (m, 1H), 2.13 (s, 3H), 1.73 (dd, J=13.3, 2.9 Hz, 2H), 1.39-1.52 (m, 2H); LCMS RT=3.330 min, m/z 340.2 [M+H⁺]; HRMS (ESI) m/z calcd for C₁₉H₂₆N₅O [M+H⁺] 340.2137.

### Example 148

### 1-(5-(3,4-Dihydro-2H-Benzo[B][1,4]Dioxepin-7-yl)Thiazol-2-yl)Piperidine-4-Carboxylic Acid (XJB04-008)

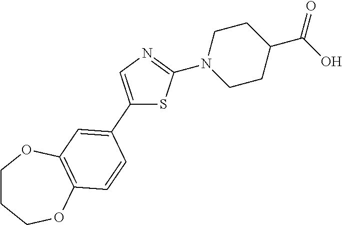 [SMILES: O=C(O)C1CCN(c2ncc(-c3ccc4c(c3)OCCCO4)s2)CC1]

LiOH (86.0 mg, 3.60 mmol) was added to a solution of ethyl 1-(5-(3,4-dihydro-2H-benzo[b][1,4]dioxepin-7-yl)thiazol-2-yl)piperidine-4-carboxylate (284 mg, 0.72 mmol) in THF (6.00 mL) and H₂O (2.00 mL) was added at room temperature. The reaction mixture was stirred at room temperature for 24 hours, diluted with 100 mL CH₂Cl₂ and washed with 2 N HCl (25.0 mL). The organic layer was separated, dried and concentrated as a yellow oil. The crude was purified by Biotage with 0-15% methanol in CH₂Cl₂ to give 233 mg (90%) product as a white solid: ¹H NMR (400 MHz, DMSO-d₆) δ ppm 12.32 (br. s., 1H), 7.48 (s, 1H), 7.07 (d, J=2.2 Hz, 1H), 6.97-7.03 (m, 1H), 6.89-6.96 (m, 1H), 4.12 (dt, J=9.2, 5.5 Hz, 4H), 3.83 (ddd, J=13.2, 3.7, 3.5 Hz, 2H), 2.98-3.20 (m, 2H), 2.51-2.57 (m, 1H), 2.04-2.17 (m, 2H), 1.87-1.98 (m, 2H), 1.49-1.69 (m, 2H); LCMS RT=4.177 min, m/z 361.1 [M+H⁺]; HRMS (ESI) m/z calcd for C₁₈H₂₁N₂O₄S [M+H⁺] 361.1222.

### Example 149

### 1-(4-(3-(Dimethylamino)Phenyl)-5-Fluoropyrimidin-2-yl)Piperidine-4-Carboxamide (XJB04-009-C)

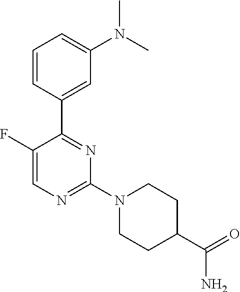 [SMILES: CN(C)c1cccc(-c2nc(N3CCC(C(N)=O)CC3)ncc2F)c1]

The compound was prepared according to the general protocol A: ¹H NMR (400 MHz, DMSO-d₆) δ ppm 8.46 (d, J=3.5 Hz, 1H), 7.17-7.45 (m, 4H), 6.91 (dd, J=8.3, 2.1 Hz, 1H), 6.77 (br. s., 1H), 4.50-4.77 (m, 2H), 2.87-3.03 (m, 8H), 2.38 (tt, J=11.5, 3.9 Hz, 1H), 1.77 (dd, J=13.5, 3.1 Hz, 2H), 1.51 (qd, J=12.4, 4.2 Hz, 2H); LCMS RT=3.990 min, m/z 344.2 [M+H⁺]; HRMS (ESI) m/z calcd for C₁₈H₂₃FN₅O [M+H⁺] 344.1887.

### Example 150

### 1-(5-(3,4-Dimethoxyphenyl)Thiazol-2-yl)Piperidine-4-Carboxamide (XJB04-010-C)

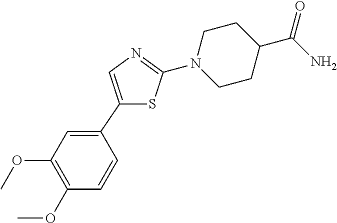 [SMILES: COc1ccc(-c2cnc(N3CCC(C(N)=O)CC3)s2)cc1OC]

The compound was prepared according to the general protocol A: ¹H NMR (400 MHz, DMSO-d₆) δ ppm 7.47 (s, 1H), 7.31 (br. s., 1H), 7.05 (s, 1H), 6.93 (d, J=1.2 Hz, 2H), 6.82 (br. s., 1H), 3.90 (ddd, J=12.7, 3.1, 2.9 Hz, 2H), 3.79 (s, 3H), 3.75 (s, 3H), 3.04 (td, J=12.5, 2.9 Hz, 2H), 2.35 (tt, J=11.4, 3.7 Hz, 1H), 1.79 (dd, J=13.2, 2.8 Hz, 2H), 1.52-1.68 (m, 2H); LCMS RT=3.552 min, m/z 348.1 [M+H⁺]; HRMS (ESI) m/z calcd for C₁₇H₂₂N₃O₃S [M+H⁺] 348.1382.

### Example 151

### 1-(5-(3,4-Dihydro-2H-Benzo[B][1,4]Dioxepin-7-yl)Thiazol-2-yl)-N-Phenylpiperidine-4-Carboxamide (XJB04-011-C)

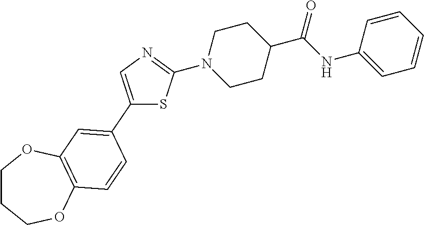 [SMILES: O=C(Nc1ccccc1)C1CCN(c2ncc(-c3ccc4c(c3)OCCCO4)s2)CC1]

The compound was prepared according to the general protocol A: ¹H NMR (400 MHz, DMSO-d₆) δ ppm 9.95 (s, 1H), 7.60 (dd, J=8.7, 1.1 Hz, 2H), 7.49 (s, 1H), 7.24-7.33 (m, 2H), 6.97-7.11 (m, 3H), 6.92-6.97 (m, 1H), 4.13 (dt, J=9.4, 5.5 Hz, 4H), 3.90-4.03 (m, 2H), 3.10 (td, J=12.6, 2.7 Hz, 2H), 2.62 (tt, J=11.5, 3.7 Hz, 1H), 2.10 (quin, J=5.4 Hz, 2H), 1.90 (dd, J=13.0, 2.8 Hz, 2H), 1.71 (qd, J=12.4, 4.3 Hz, 2H); LCMS RT=4.936 min, m/z 436.2 [M+H⁺]; HRMS (ESI) m/z calcd for C₂₄H₂₆N₃O₃S [M+H⁺] 436.1695.

### Example 152

### 1-(2-(3-(Dimethylamino)Phenyl)-5-Fluoropyrimidin-4-yl)Piperidine-4-Carboxamide (XJB04-013)

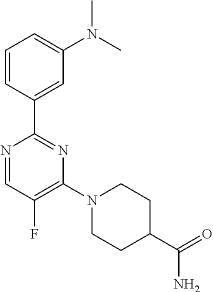 [SMILES: CN(C)c1cccc(-c2ncc(F)c(N3CCC(C(N)=O)CC3)n2)c1]

The compound was prepared according to the general protocol A: ¹H NMR (400 MHz, DMSO-d₆) δ ppm 8.32 (d, J=6.8 Hz, 1H), 7.66 (dd, J=2.5, 1.4 Hz, 1H), 7.51-7.60 (m, 1H), 7.30 (br. s., 1H), 7.27 (t, J=8.0 Hz, 1H), 6.84 (ddd, J=8.3, 2.7, 0.9 Hz, 1H), 6.80 (br. s., 1H), 4.39-4.55 (m, 2H), 3.07-3.20 (m, 2H), 2.95 (s, 6H), 2.41-2.48 (m, 1H), 1.84 (dd, J=13.2, 3.0 Hz, 2H), 1.53-1.71 (m, 2H); LCMS RT=3.373 min, m/z 344.2 [M+H⁺]; HRMS (ESI) m/z calcd for C₁₈H₂₃FN₅O [M+H⁺] 344.1887.

### Example 153

### 1-(5-(2,2-Difluorobenzo[D][1,3]Dioxol-5-yl)Thiazol-2-yl)Piperidine-4-Carboxamide (XJB04-025)

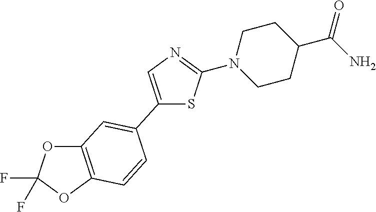 [SMILES: NC(=O)C1CCN(c2ncc(-c3ccc4c(c3)OC(F)(F)O4)s2)CC1]

The compound was prepared according to the general protocol A as a TFA salt: ¹H NMR (400 MHz, DMSO-d₆) δ ppm 7.63 (dd, J=1.8, 0.4 Hz, 1H), 7.61 (s, 1H), 7.38 (dd, J=8.4, 0.4 Hz, 1H), 7.32 (br. s., 1H), 7.22 (dd, J=8.3, 1.9 Hz, 1H), 6.82 (br. s., 1H), 3.91 (dt, J=12.9, 3.2 Hz, 2H), 3.08 (td, J=12.5, 2.9 Hz, 2H), 2.37 (tt, J=11.5, 3.7 Hz, 1H), 1.80 (dd, J=13.5, 3.3 Hz, 2H), 1.60 (qd, J=12.4, 4.4 Hz, 2H); ¹⁹F NMR (376 MHz, DMSO-d₆) δ ppm −49.34 (s), −73.52 (s); LCMS RT=4.509 min, m/z 368.1 [M+H⁺]; HRMS (ESI) m/z calcd for C₁₆H₁₆F₂N₃O₃S [M+H⁺] 368.0880.

### Example 154

### 5-(2-Fluorobenzyloxy)Quinazoline-2,4-Diamine (XJB04-062)

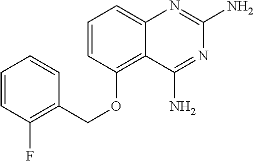 [SMILES: Nc1nc(N)c2c(OCc3ccccc3F)cccc2n1]

The compound was prepared according to the literature: ¹H NMR (400 MHz, DMSO-d₆) δ ppm 7.61 (td, J=7.4, 1.8 Hz, 1H), 7.42-7.52 (m, 1H), 7.20-7.41 (m, 3H), 7.12 (br. s., 2H), 6.79 (dd, J=8.4, 1.0 Hz, 1H), 6.67 (dd, J=8.0, 1.0 Hz, 1H), 5.94 (br. s., 2H), 5.32 (s, 2H); ¹⁹F NMR (376 MHz, DMSO-d₆) δ ppm −118.14-−118.22 (m); LCMS RT=3.896 min, m/z 285.1 [M+H⁺]; HRMS (ESI) m/z calcd for C₁₅H₁₄FN₄O [M+H⁺] 285.1152.

### Example 155

### 5-((1-(2-Fluorobenzyl)Piperidin-4-yl)Methoxy)Quinazoline-2,4-Diamine (XJB04-067)

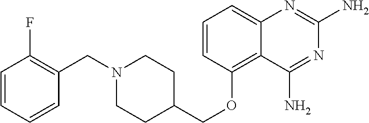 [SMILES: Nc1nc(N)c2c(OCC3CCN(Cc4ccccc4F)CC3)cccc2n1]

The compound was prepared according to the literature: ¹H NMR (400 MHz, DMSO-d₆) δ ppm 7.37-7.44 (m, 1H), 7.27-7.37 (m, 2H), 7.10-7.22 (m, 4H), 6.76 (dd, J=8.4, 0.8 Hz, 1H), 6.53 (dd, J=8.0, 0.8 Hz, 1H), 5.93 (br. s., 2H), 3.98 (d, J=6.3 Hz, 2H), 3.53 (br. s., 2H), 2.78-2.94 (m, 2H), 1.95-2.07 (m, 2H), 1.85 (br. s., 1H), 1.67-1.78 (m, 2H), 1.25-1.44 (m, 2H); ¹⁹F NMR (376 MHz, DMSO-d₆) δ ppm −118.13-−118.19 (m); LCMS RT=3.128 min, m/z 382.2 [M+H⁺]; HRMS (ESI) m/z calcd for C₂₁H₂₅FN₅O [M+H⁺] 382.2043.

### Example 156

### 1-(5-(3,4-Dihydro-2h-benzo[b][1,4]dioxepin-7-yl)-4-oxo-4,5-Dihydrothiazol-2-yl)Piperidine-4-Carboxamide

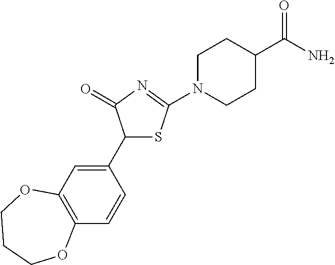 [SMILES: NC(=O)C1CCN(C2=NC(=O)C(c3ccc4c(c3)OCCCO4)S2)CC1]

A solution of 1-(5-(3,4-dihydro-2H-benzo[b][1,4]dioxepin-7-yl)thiazol-2-yl)piperidine-4-carboxamide (10.0 mg, 0.028 mmol) in acetonitrile (1.00 mL) was treated at room temperature with AccuFluor (10.74 mg, 0.033 mmol). The reaction mixture was stirred at 60° C. for 2 h, quenched with water and extracted with CH₂Cl₂. The organic layer was separated, dried Na₂SO₄ and concentrated as yellow crude foam. The crude mixture was purified by Biotage with 0-20% MeOH in CH₂Cl₂ to give 4.3 mg (41%) product as a colorelss form: ¹H NMR (400 MHz, DMSO-d₆) δ ppm 7.31 (br. s., 1H), 6.87-6.96 (m, 1H), 6.70-6.88 (m, 3H), 5.41 (s, 1H), 4.49 (t, J=13.2 Hz, 1H), 4.08 (t, J=5.0 Hz, 4H), 3.60-3.76 (m, 1H), 3.29-3.44 (m, 1H), 3.16-3.26 (m, 1H), 2.38-2.47 (m, 1H), 2.01-2.13 (m, 2H), 1.72-1.93 (m, 2H), 1.44-1.72 (m, 2H); LCMS RT=3.768 min, m/z 376.1 [M+H⁺].

### Example 157

### 1-(5-(3-(Trifluoromethoxy)Phenyl)Thiazol-2-yl)Piperidine-4-Carboxamide (XJB07-003)

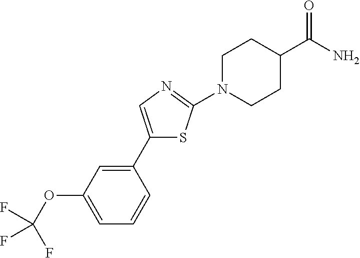 [SMILES: NC(=O)C1CCN(c2ncc(-c3cccc(OC(F)(F)F)c3)s2)CC1]

The compound was prepared according to the general protocol A: ¹H NMR (400 MHz, DMSO-d₆) δ ppm 7.72 (s, 1H), 7.35-7.54 (m, 3H), 7.29 (s, 1H), 7.10-7.22 (m, 1H), 6.80 (br. s., 1H), 3.75-4.01 (m, 2H), 3.07 (td, J=12.7, 3.5 Hz, 2H), 2.25-2.40 (m, 1 H), 1.78 (dd, J=13.5, 3.9 Hz, 2H), 1.48-1.67 (m, 2H); ¹⁹F NMR (376 MHz, DMSO-d₆) δ ppm −56.66 (s); LCMS RT=4.645 min, m/z 372.1 [M+H⁺].

### Example 158

### 1-(5-(4-(Trifluoromethoxy)Phenyl)Thiazol-2-yl)Piperidine-4-Carboxamide (XJB07-004)

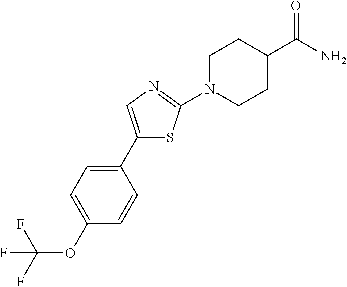 [SMILES: NC(=O)C1CCN(c2ncc(-c3ccc(OC(F)(F)F)cc3)s2)CC1]

The compound was prepared according to the general protocol A: ¹H NMR (400 MHz, DMSO-d₆) δ ppm 7.62 (s, 1H), 7.52-7.58 (m, 2H), 7.32 (d, J=8.6 Hz, 2H), 7.29 (br. s., 1H), 6.80 (br. s., 1H), 3.89 (dt, J=13.4, 3.1 Hz, 2H), 3.07 (td, J=12.8, 3.2 Hz, 2H), 2.24-2.40 (m, 1H), 1.78 (dd, J=13.4, 2.6 Hz, 2H), 1.46-1.68 (m, 2H); ¹⁹F NMR (376 MHz, DMSO-d₆) δ ppm −56.90 (s); LCMS RT=4.565 min, m/z 372.1 [M+H⁺].

### Example 159

### 1-(5-(3-(Trifluoromethylthio)Phenyl)Thiazol-2-yl)Piperidine-4-Carboxamide (XJB07-0151

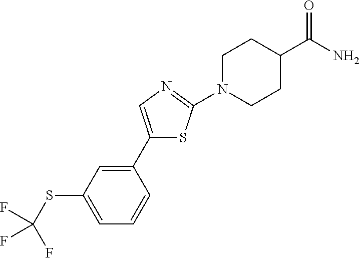 [SMILES: NC(=O)C1CCN(c2ncc(-c3cccc(SC(F)(F)F)c3)s2)CC1]

The compound was prepared according to the general protocol A: ¹H NMR (400 MHz, DMSO-d₆) δ ppm 7.70-7.86 (m, 2H), 7.64 (dq, J=6.5, 2.0 Hz, 1H), 7.36-7.56 (m, 2H), 7.29 (br. s., 1H), 6.79 (br. s., 1H), 3.81-3.97 (m, 2H), 2.97-3.16 (m, 2H), 2.24-2.39 (m, 1H), 1.77 (dd, J=13.5, 2.7 Hz, 2H), 1.45-1.67 (m, J=12.3, 12.3, 12.1, 4.5 Hz, 2 H); ¹⁹F NMR (376 MHz, DMSO-d₆) δ ppm −41.88 (s); LCMS RT=4.862 min, m/z 388.0 [M+H⁺].

### Example 160

### 1-(5-(3-(Methylthio)Phenyl)Thiazol-2-yl)Piperidine-4-Carboxamide (XJB07-016)

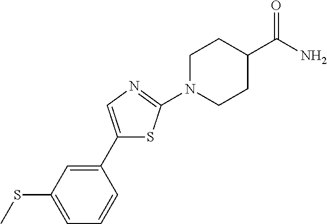 [SMILES: CSc1cccc(-c2cnc(N3CCC(C(N)=O)CC3)s2)c1]

The compound was prepared according to the general protocol A: ¹H NMR (400 MHz, DMSO-d₆) δ ppm 7.64 (s, 1H), 7.23-7.33 (m, 3H), 7.16-7.21 (m, 1H), 7.08 (d, J=7.8 Hz, 1H), 6.80 (br. s., 1H), 3.81-4.02 (m, 2H), 3.07 (td, J=12.5, 2.8 Hz, 2H), 2.47 (s, 3H), 2.24-2.39 (m, J=11.4, 11.4, 4.0, 3.8 Hz, 1H), 1.78 (dd, J=13.2, 2.8 Hz, 2H), 1.46-1.67 (m, 2H); LCMS RT=4.129 min, m/z 334.1 [M+H⁺].

### Example 161

### 1-(5-(4-(Trifluoromethylthio)Phenyl)Thiazol-2-yl)Piperidine-4-Carboxamide (XJB07-017)

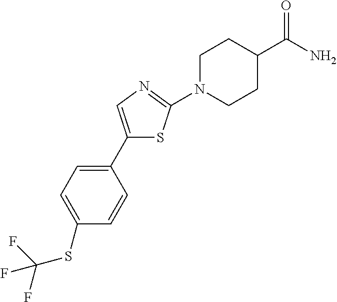 [SMILES: NC(=O)C1CCN(c2ncc(-c3ccc(SC(F)(F)F)cc3)s2)CC1]

The compound was prepared according to the general protocol A: LCMS RT=4.997 min, m/z 388.0 [M+H⁺].

### Example 162

### 1-(5-(3-Methoxyphenyl)Thiazol-2-yl)Piperidine-4-Carboxamide (XJB07-021)

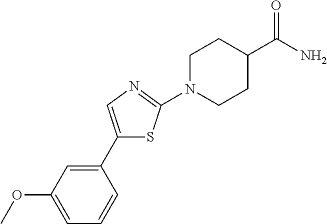 [SMILES: COc1cccc(-c2cnc(N3CCC(C(N)=O)CC3)s2)c1]

The compound was prepared according to the general protocol A: ¹H NMR (400 MHz, DMSO-d₆) δ ppm 7.59 (s, 1H), 7.30 (br. s., 1H), 7.24 (t, J=7.9 Hz, 1H), 6.95-7.03 (m, 2H), 6.80 (br. s., 1H), 6.77 (ddd, J=8.2, 2.3, 1.0 Hz, 1H), 3.90 (ddd, J=12.9, 3.4, 3.2 Hz, 2H), 3.76 (s, 3H), 3.05 (td, J=12.5, 2.8 Hz, 2H), 2.34 (tt, J=11.5, 3.7 Hz, 1H), 1.78 (dd, J=13.5, 3.3 Hz, 2H), 1.46-1.69 (m, 2H); LCMS RT=3.786 min, m/z 318.1 [M+H⁺].

### Example 163

### 1-(5-(4-Methoxyphenyl)Thiazol-2-yl)Piperidine-4-Carboxamide (XJB07-022)

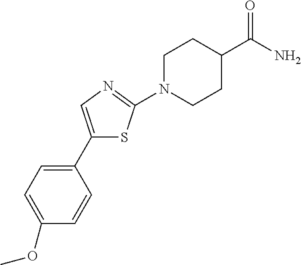 [SMILES: COc1ccc(-c2cnc(N3CCC(C(N)=O)CC3)s2)cc1]

The compound was prepared according to the general protocol A: LCMS RT=3.700 min, m/z 318.1 [M+H⁺].

### Example 164

### Quantitative High Throughput Screening (qHTS) of Compounds

A cell-based quantitative high throughput screening (qHTS) assay is used to identify compounds able to increase SMN production. The assay uses a SMN2-luciferase reporter to determine if exon 7 of the SMN gene is expressed, which correlates with increased full-length SMN production from the SMN2 gene. As controls, a homologue SMN1-luciferase reporter cell line looks for variations in expression of the SMN1 gene and a cell line with just the luciferase reporter looks at the capacity of the compounds to inhibit luciferase. In addition, increments in SMN production by lead compounds were confirmed by Western-Blot analysis using SMA patient fibroblasts.

For the qHTS assay, concentration-response profiles are generated at the primary-screen level for each library compound to minimize the number of false positives. A dilution series consisting of seven points separated by five-fold concentration differences is prepared for each compound, yielding a concentration series that spans a range of about four orders of magnitude, for example from a high concentration of 50 micromolar to a low concentration of 3.2 nanomolar and providing adequate data for fitting to a Hill equation. For the assay, the SMN2 reporter cell line was plated at a density of ˜2000 cells/well in complete media (phenol red free DMEM, 10% detacl calf serum, and 1× pen/strep), in 1536 well white solid bottom tissue culture treated plates (Greiner). Cells were allowed to adhere and recover at 37 C in 5% CO₂ for 10-12 hours. Compounds were added to the plates by a Pintool (Kalypsys) at a volume of 23 nl/well. Cells were incubated in the presence of compounds for 2 days at 37 C in 5% CO₂. In order to quantify luciferase reporter levels, 3 ul/well of OneGlo (Promega) was added to each well and incubated at room temperature for 10 minutes. Luminescence from the reporter in each well is measured using a Viewlux CCD based imager (Perkin Elmer) with an integration time of 30 seconds and a binning of 2× in luminescence mode. Sodium butyrate was used as the positive control compound for luciferase induction and a column of 32 wells of 9.2 mM compound on each plate was used to monitor assay performance.

### Reporter System

The SMN reporters used herein are modified from those previously described (M. L. Zhang, C. L. Lorson, E. J. Androphy and J. Zhou, An in vivo reporter system for measuring increased inclusion of exon 7 in SMN2 mRNA: potential therapy of SMA, Gene Ther 8 (2001), pp. 1532-1538.)

The sequence of the SMN1 reporter vector used herein is given in SEQ ID NO.:1, while that of the SMN2 reporter vector is given in SEQ ID NO.:2.

Each of the SMN reporters is constructed from three separate DNA fragments: (1) an SMN promotor sequence from the transcriptional reporter p 4.0T (SMN1) or p3.4C (SMN2) described in Monani, et al. Biochimica et Biophysica Acta 1445 (1999) 330-336; (2) a modification of the firefly luciferase splicing reporter for SMN1 or SMN2 described by Zhang et al; and (3) a 744 bp SMN cDNA fragment.

The 3.7 kB SMN1 promoter fragment is obtained by restriction of p4.0T with BglII and SacII. A 3.4 kB SMN2 promoter fragment is obtained by restriction of p3.4C with KpnI and SacII; a 300 bp BglII/KpnI fragment from p4.0T is then added to the 3.4 kB SMN2 promoter fragment to yield the final 3.7 kB SMN2 promoter fragment.

The modified SMN1 and SMN2 firefly luciferase splicing reporters are obtained by removing 4 kB of intron 6 in the original reporter system by restriction with SmaI/Swa followed by religation of the reporter. A 4 kB luciferase splicing reporter is then obtaind by digestion with XhoI and NotI.

Ths SMN cDNA fragment is obtained by generating the productof a polymerase chain reaction (PCR) on a cDNA preparation using CACCCGCGGGTTTGCTATGGCGATGAGCAGCGGC (SEQ ID NO:3) as the forward primer and Tatctcgagtggtccagaaggaaatggaggcagcc (SEQ ID NO:4) as the reverse primer. The PCR product is then digested with XhoI and SacII.

To obtain the SMN1 or the SMN2 reporter, the appropriate three fragments are ligated into pIRES (BD Clontech) vector at the BglII and NOTI sites, removing the CMV promoter present in pIRES. The entire reporter for SMN1 or SMN2 is then removed from the ORES vector by digestion with Acc651, yielding a 9 kB fragment including the promoter, cDNA fragment, and the modified luciferase reporter, as well as the 3′ UTR sequences of the pIRES expression unit.

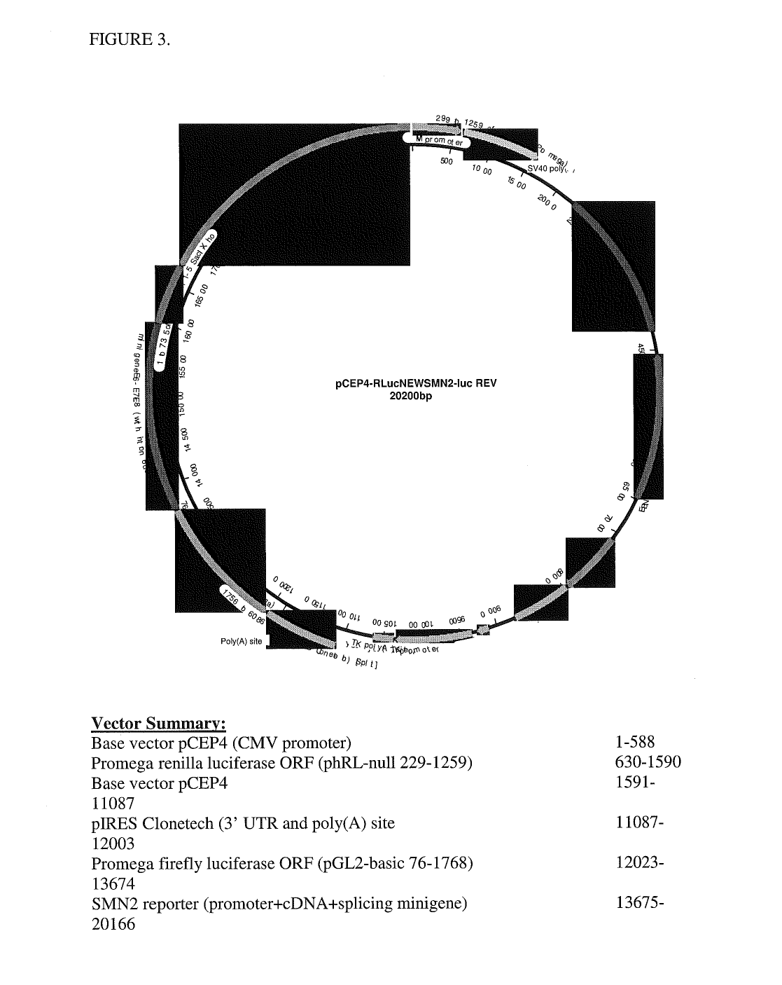

The 9 kB reporter fragments for SMN1 or SMN2 is cloned into the BsrGI site of pCEP-4 R-luc, which expresses renilla luciferase from its CMV promoter. The SMN-luciferase reporter and the renilla luciferase reporter have opposite orientation to each other in the final constructs. A map of the SMN2 reporter construct is shown in FIG. 3.

The SMN1 and SMN 2 reporter constructs were each transfected by lipofectamine 2000 into HEK 293 cells. Cells were selected for 2 weeks with 300 ug/mL Hygromycin B. The selected cells were diluted and allowed to grow into monoclonal populations.

These monoclonal populations were tested for firefly (SMN reporter) and renilla luciferase expression. One population of SMN1 (A3) expressing cells and three populations of SMN2 (B3, B4, and B5) expression cells were isolated.

### Cell Culture

Cells are incubated at 37° C. with 5% CO2.

HEK-293 cells are grown in Dulbecco's Modified Eagle Medium (D-MEM) (Gibco 11995) with 10% fetal bovine serum (FBS Atlas) and 1× Penicillin-Streptomycin (1× pen-strep) (Gibco 15140 as 100×). Reporter cell lines containing SMN1, SMN2, or control luciferase reporter are selected and maintained in D-MEM with 10% FBS and 1× pen-strep with 200 μg/mL Hygromycin B (Invitrogen 10687-010).

Type I spinal muscular atrophy (SMA)-affected human primary fibroblasts (#GM03813) and carrier parents (#GM03814, mother and #GM03815, father) are obtained live with low passage number from Coriell Cell Repositories. The primary human fibroblasts are grown in D-MEM with 10% FBS and 1× pen-strep.

### Western Blotting (Immunoblotting)

For detection of SMN protein in patient fibroblasts, 8,000 cells per cm² are plated 24 hours prior to drug addition. Fresh media and compound are added every 24 hours. After 72 hours, cells are harvested, washed with cold PBS, and lysed. Samples are subjected to polyacrylamide gel electrophoresis (PAGE). It is determined that 10 μg total protein per lane is within the linear range for immunoblot detection of SMN and alpha-tubulin.

Western blots of the gel are probed for SMN with the 4B7 mouse monoclonal antibody (from the lab of Christian Lorson). A goat anti-mouse antibody (Santa Cruz Biotechnology/#sc-2005) conjugated to horseradish peroxidase (HRP) is used as the secondary antibody. Quantification of protein is performed by detecting chemiluminescence with a Fujifilm LAS-4000 Multifunctional Imaging System.

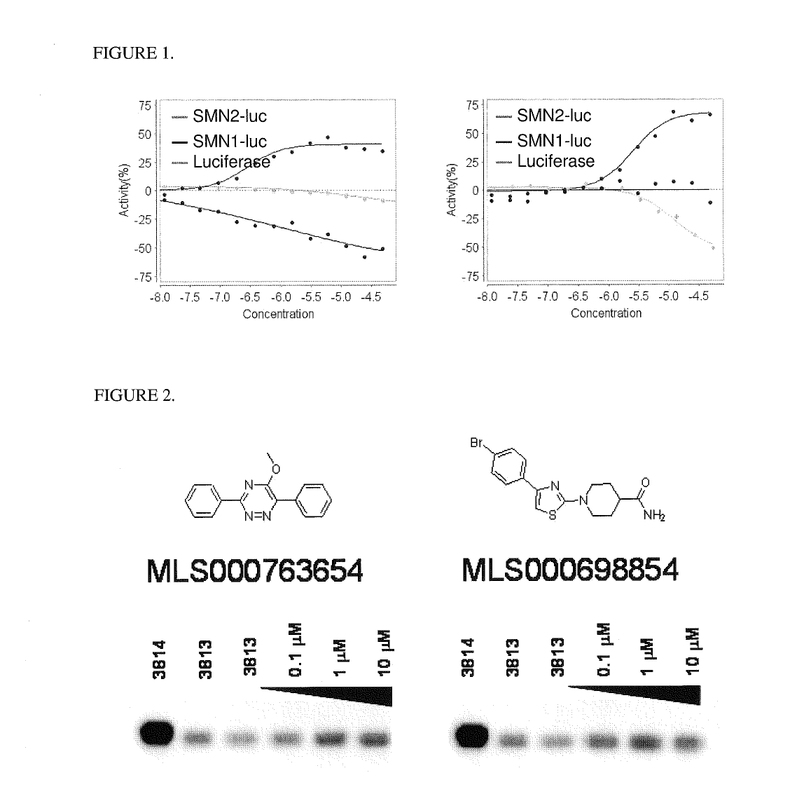

Several HTS hits are evaluated and confirmed, both in primary and orthogonal assays. FIGS. 1 and 2 show results for two compounds identified in the screen.

FIG. 1 presents the percent luciferase activity as a function of concentration of compounds MLS0006988454 (right panel) and MLS000763654 (left panel). MLS000698854 increases the expression of SMN2 and does not affect the expression of SMN1, while MLS000763654 increases the expression of SMN2 and reduces the expression of SMN1. FIG. 1 also shows the difference in luciferase inhibitory capacity of MLS000763654 and MLS000698854.

FIG. 2 shows an image of the Western blot analysis of SMN protein obtained from a sample of SMA patient cells (3813) cultured with either MLS00069884 or MLS000763654. Cells incubated with either compound at a concentration of 0.1-10 μM show a concentration-dependent increase in SMN protein from the level observed in the absence of the test compound. For comparison, an analogous cell sample from the SMA-carrier mother (3814) of SMA patient 3813 is analyzed for SMN protein content. The SMA patient shows a much smaller amount of SMN protein present in the cell sample.

### Example 165

### Biological Activity of Exemplified Compounds

The compounds listed in Table 1 were tested in the luciferase assay described in Example 164 and found to modulate levels of the full length SMN2 gene. The column labeled “increased SMN2 activity” reports the percent increase (or decrease) in SMN2 levels compared to control in which no test compound is added. Curve class indicates the agonist/antagonist character of the compound. A curve class of 1.1 is a full agonist/antagonist; a curve class of 4 indicates the compound is inactive.

Curve class indicates the agonist/antagonist character of the compound's concentration-response. A curve class of 1.1 is a full agonist/antagonist; a curve class of 4 indicates the compound is inactive. Lower values indicate a more significant concentration-response. Details on curve class methodology can be found in: Southall, N T, Jadhav A, Huang R, Nguyen T, Wang Y. Enabling the Large Scale Analysis of Quantitative High Throughput Screening Data. In Handbook of Drug Screening, Second Edition; Seethala R, Zhang L, Eds.; Taylor and Francis: New York, 2009, 442-463. ISBN: 1420061682.”

TABLE 1

| Example | Curve | Increased | |
|---|---|---|---|
| No. | Class | SMN2 activity | Sample ID |
| 18 | 2.2 | 48.75 | NCGC00183569- |
| | | | 01 |
| 19 | 1.1 | 287.7 | NCGC00183531- |
| | | | 01 |
| 20 | 1.1 | 380.5714286 | NCGC00183541- |
| | | | 01 |
| 21 | 1.1 | 439.1428571 | NCGC00183582- |
| | | | 01 |
| 22 | 1.1 | 386.1 | NCGC00183527- |
| | | | 01 |
| 23 | 1.1 | 267.7093911 | NCGC00183533- |
| | | | 01 |
| 25 | 2.1 | 209.1 | NCGC00183548- |
| | | | 01 |
| 26 | 2.2 | 57.54761905 | NCGC00183547- |
| | | | 01 |
| 28 | 2.2 | 58.07142857 | NCGC00183550- |
| | | | 01 |
| 29 | 2.1 | 254.2857143 | NCGC00183551- |
| | | | 01 |
| 30 | 1.1 | 166.6285714 | NCGC00183552- |
| | | | 01 |
| 33 | 2.2 | 64 | NCGC00183539- |
| | | | 01 |
| 34 | 2.2 | 53.22619048 | NCGC00183571- |
| | | | 01 |
| 35 | 1.1 | 357 | NCGC00183572- |
| | | | 01 |
| 37 | 2.4 | 20.28571429 | NCGC00183574- |
| | | | 01 |
| 38 | 1.1 | 274.5714286 | NCGC00183540- |
| | | | 01 |
| 39 | 2.2 | 57.60714286 | NCGC00183542- |
| | | | 01 |
| 32 | 2.1 | 272.5714286 | NCGC00183536- |
| | | | 01 |
| 44 | 1.1 | 550.2857143 | NCGC00183535- |
| | | | 01 |
| 45 | 2.1 | 130.3714286 | NCGC00183575- |
| | | | 01 |
| 46 | 2.1 | 187.4285714 | NCGC00183576- |
| | | | 01 |
| 47 | 1.1 | 359.16 | NCGC00183577- |
| | | | 01 |
| 48 | 1.1 | 459 | NCGC00183579- |
| | | | 01 |
| 50 | 2.1 | 589.68 | NCGC00183580- |
| | | | 01 |
| 51 | 2.2 | 50.92857143 | NCGC00183581- |
| | | | 01 |
| 53 | 3 | 79.57142858 | NCGC00183555- |
| | | | 01 |
| 55 | 1.1 | 356 | NCGC00183556- |
| | | | 01 |
| 56 | 1.1 | 162 | NCGC00183537- |
| | | | 01 |
| 60 | 2.1 | 100 | NCGC00183560- |
| | | | 01 |
| 62 | 2.2 | 51.17857143 | NCGC00183565- |
| | | | 01 |
| 63 | 1.1 | 504 | NCGC00183564- |
| | | | 01 |
| 64 | 2.1 | 369 | NCGC00183538- |
| | | | 01 |
| 65 | 1.1 | 534 | NCGC00183562- |
| | | | 01 |
| 68 | 2.1 | 100 | NCGC00183567- |
| | | | 01 |
| 69 | 1.1 | 554.58 | NCGC00183568- |
| | | | 01 |
| 69 | 1.1 | 554.58 | NCGC00183568- |
| | | | 01 |
| 70 | 2.1 | 101.8571429 | NCGC00184909- |
| | | | 01 |
| 71 | 2.4 | 42.71428572 | NCGC00184910- |
| | | | 01 |
| 72 | 1.1 | 262.4 | NCGC00184911- |
| | | | 01 |
| 74 | 2.2 | 77.47826087 | NCGC00184913- |
| | | | 01 |
| 77 | 2.4 | 31.28985507 | NCGC00184916- |
| | | | 01 |
| 80 | 1.1 | 387 | NCGC00184923- |
| | | | 01 |
| 81 | 2.2 | 100 | NCGC00184924- |
| | | | 01 |
| 83 | 2.4 | 58.51268116 | NCGC00184926- |
| | | | 01 |
| 84 | 2.2 | 73.33333333 | NCGC00184927- |
| | | | 01 |
| 85 | 1.4 | 44.76811594 | NCGC00184928- |
| | | | 01 |
| 87 | 1.1 | 598.6956522 | NCGC00184930- |
| | | | 01 |
| 88 | 1.1 | 550.44 | NCGC00184931- |
| | | | 01 |
| 89 | 1.1 | 236.25 | NCGC00184932- |
| | | | 01 |
| 90 | 1.1 | 446.3478261 | NCGC00184933- |
| | | | 01 |
| 91 | 1.1 | 178.2 | NCGC00184936- |
| | | | 01 |
| 92 | 1.1 | 240 | NCGC00184937- |
| | | | 01 |
| 93 | 2.1 | 401.7391304 | NCGC00184938- |
| | | | 01 |
| 95 | 1.1 | 296.7652174 | NCGC00184940- |
| | | | 01 |
| 96 | 1.1 | 211.6173913 | NCGC00184941- |
| | | | 01 |
| 98 | 1.1 | 243.3391304 | NCGC00184943- |
| | | | 01 |
| 99 | 1.1 | 443.1652174 | NCGC00184944- |
| | | | 01 |
| 100 | 2.2 | 64.26086957 | NCGC00184945- |
| | | | 01 |
| 102 | 2.2 | 77.86956522 | NCGC00184947- |
| | | | 01 |
| 104 | 2.1 | 306.2869565 | NCGC00184949- |
| | | | 01 |
| 106 | 1.2 | 54 | NCGC00184952- |
| | | | 01 |
| 107 | 1.1 | 605.5826087 | NCGC00184953- |
| | | | 01 |
| 108 | −2.1 | −97.2921457 | NCGC00184954- |
| | | | 01 |
| 110 | 1.1 | 234.5250202 | NCGC00184956- |
| | | | 01 |
| 111 | 1.1 | 182.5622988 | NCGC00184957- |
| | | | 01 |
| 112 | 2.1 | 66.76623905 | NCGC00184958- |
| | | | 01 |
| 113 | −1.4 | −56.61710305 | NCGC00184959- |
| | | | 01 |
| 118 | 2.1 | 88.78623188 | NCGC00184964- |
| | | | 01 |
| 119 | 1.1 | 609.3913044 | NCGC00187892- |
| | | | 01 |
| 120 | 1.1 | 424.5652174 | NCGC00187849- |
| | | | 01 |
| 121 | 1.1 | 601.5130435 | NCGC00187850- |
| | | | 01 |
| 122 | 1.1 | 498.2522435 | NCGC00187893- |
| | | | 01 |
| 123 | 1.1 | 709.5652174 | NCGC00187894- |
| | | | 01 |
| 124 | 1.1 | 370.5 | NCGC00187851- |
| | | | 01 |
| 125 | 1.1 | 589.875 | NCGC00187852- |
| | | | 01 |
| 126 | 1.1 | 444.6 | NCGC00187895- |
| | | | 01 |
| 127 | 1.1 | 298.225 | NCGC00187896- |
| | | | 01 |
| 128 | 1.1 | 234.1565217 | NCGC00187853- |
| | | | 01 |
| 129 | 1.1 | 364.3043478 | NCGC00187854- |
| | | | 01 |
| 130 | 1.1 | 257.1130435 | NCGC00187897- |
| | | | 01 |
| 132 | 1.1 | 448.4687497 | NCGC00187856- |
| | | | 01 |
| 133 | 1.2 | 54 | NCGC00187857- |
| | | | 01 |
| 134 | 1.1 | 576.0575669 | NCGC00187898- |
| | | | 01 |
| 135 | 1.1 | 129.4281181 | NCGC00187858- |
| | | | 01 |
| 136 | 1.1 | 188.1723348 | NCGC00187899- |
| | | | 01 |
| 137 | 1.1 | 685.0547232 | NCGC00187859- |
| | | | 01 |
| 138 | 1.1 | 565.616596 | NCGC00187860- |
| | | | 01 |
| 139 | 1.1 | 275.2109951 | NCGC00187861- |
| | | | 01 |
| 141 | 1.1 | 224.6223263 | NCGC00187865- |
| | | | 01 |
| 142 | 1.1 | 211.1200581 | NCGC00187866- |
| | | | 01 |
| 143 | 2.1 | 81.27122 | NCGC00187867- |
| | | | 01 |
| 144 | 1.1 | 186.2189574 | NCGC00187868- |
| | | | 01 |
| 145 | 1.1 | 199.9448976 | NCGC00187869- |
| | | | 01 |
| 146 | 1.1 | 319.5582815 | NCGC00187870- |
| | | | 01 |
| 148 | 1.1 | 325.6039419 | NCGC00187872- |
| | | | 01 |
| 149 | 1.1 | 373.2079249 | NCGC00187873- |
| | | | 01 |
| 150 | 1.1 | 209.7030435 | NCGC00187874- |
| | | | 01 |
| 151 | 1.1 | 352.9678696 | NCGC00187875- |
| | | | 01 |
| 152 | 1.1 | 141.780365 | NCGC00187876- |
| | | | 01 |
| 153 | 1.1 | 598.3851525 | NCGC00187900- |
| | | | 01 |

### Example 166

### Permeability of Selected Compounds in CaCo-2 Cells

CaCo-2 cells grown in tissue culture flasks are trypsinized, suspended in medium, and the suspensions are applied to wells of a collagen-coated BioCoat Cell Environment in 24-well format (BD Biosciences) at 24,500 cells per well. The cells are allowed to grow and differentiate for three weeks, feeding at 2-day intervals.

For Apical to Basolateral (A->B) permeability, the test agent is added at 50 μM to the apical (A) side and amount of permeation is determined on the basolateral (B) side; for Basolateral to Apical (B>A) permeability, the test agent is added at 50 μM to the B side and the amount of permeation is determine on the A side. The A-side buffer contains 100 μM Lucifer yellow dye, in Transport Buffer (1.98 g/L glucose in 10 mM HEPES, 1× Hank's Balanced Salt Solution) pH 6.5, and the B-side buffer is Transport Buffer, pH 7.4. CaCo-2 cells are incubated with these buffers for 2 h., and the receiver side buffer is removed for analysis by liquid chromatography-mass spectrometry (LC/MS/MS).

Additionally, several control compounds are tested in the identical assay. Warfarin is used as a high permeability control, ranitidine is used as a low permeability control, and the Pgp inhibitor quinidine is also tested.

To verify that CaCo-2 cell monolayers are properly formed, aliquots of the cell buffers are analyzed by fluorescence to determine the transport of the impermeable dye Lucifer Yellow.

Data are expressed as permeability (Papp):

$${P}_{app} = \frac{\frac{d Q}{d t}}{{C}_{0} A}$$

where dQ/dt is the rate of permeation, C0 is the initial concentration of test agent, and A is the area of the monolayer.

In bidirectional permeability studies, the asymmetry index (AI) or efflux ratio is also calculated:

$$AI = \frac{{P}_{app} ( B \to A )}{{P}_{app} ( A \to B )}$$

An AI>1 indicated a potential substrate for PGP or other active transporters.

Samples are analyzed by LC/MS/MS using either an Agilent 6410 mass spectrometer coupled with an Agilent 1200 high performance liquid chromatography (HPLC) and a CTC PAL chilled autosampler, all controlled by MassHunter software (Agilent), or an ABI2000 mass spectrometer coupled with an Agilent 1100 HPLC and a CTC PAL chilled autosampler, all controlled by Analyst software (ABI). After separation on a C18 reverse phase HPLC column (Agilent, Waters, or equivalent) using an acetonitrile-water gradient system, peaks are analyzed by mass spectrometry (MS) using electrospray ionization (ESI) in multiple reaction monitoring (MRM) mode.

The signal is optimized for each compound by ESI positive or negative ionization mode. A MS2 selected ion monitoring (SIM) scan is used to optimize the precursor ion and a product ion analysis is used to identify the best fragment for analysis and to optimize the collision energy.

Results for compound MLS000688854, as well as for controls, are shown in the table below.

TABLE 2
Caco-2 permeability of lead compound and controls.

| | test | mean A->B | mean B->A | | |
|---|---|---|---|---|---|
| | conc | Pₐₚₚ^{a} | Pₐₚₚ^{a} | Efflux | |
| Client ID | (μM) | (10⁻⁶ cm s⁻¹) | (10⁻⁶ cm s⁻¹) | ratio^{b} | comment |
| Warfarin | 10 | 45.8 | 11.7 | 0.3 | high permeability control |
| Ranitidine | 10 | 0.42 | 2.1 | 5.0 | low permeability control |
| Quinidine | 10 | 4.2 | 24.1 | 5.7 | P-gp control |
| MLS000698854 | 10 | 32.6 | 12.0 | 0.4 | |

Table 2 shows that MLS000698854 is highly permeable in a Caco-2 cell assay, with no indication of being a Pgp substrate.

### Example 167

### Microsomal Stability of Selected Compounds

The test agent is incubated at 1 μM in duplicate with mouse microsomes at 37° C. for 60 min. The reaction contains 0.3 mg/mL microsomal protein in 100 mM potassium phosphate, 2 mM NADPH, 3 mM MgCl₂, pH 7.4. A control is run for each test agent omitting NADPH to detect NADPH-free degradation. At indicated times, an aliquot is removed from each experimental and control reaction and mixed with an equal volume of ice-cold Stop Solution (0.3% acetic acid in acetonitrile containing haloperidol, diclofenac, or other internal standard). Stopped reactions are incubated at least ten minutes at −20° C., and an additional volume of water is added. Verapamil and warfarin controls are also tested in the identical assay.

The samples are centrifuged to remove precipitated protein, and the supernatants are analyzed by LC/MS/MS, as described above, to quantitate the remaining parent. Data are reported as % remaining by dividing by the time zero concentration value.

Table 3 below shows the results of the stability testing for the control drugs and selected compounds.

TABLE 3
Stability of several analogues in mouse microsomes.

| | | | | Plus NADPH | | | Minus NADPH | | |
|---|---|---|---|---|---|---|---|---|---|
| | | | | Parent | | | Parent | | |
| | | | test | remaining | | | remaining | | |
| | | test | conc | 1^{st} | 2^{nd} | mean | 1^{st} | 2^{nd} | mean |
| structure | ID | species | (M) | (%) | (%) | (%) | (%) | (%) | (%) |
| | Verapamil | mouse | 1 | 0% | 0% | 0% | 104% | 112% | 108% |
| | Warfarin | mouse | 1 | 101% | 100% | 100% | 102% | 104% | 103% |
| 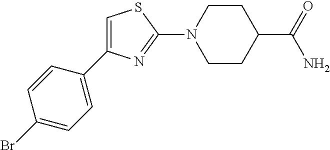 [SMILES: NC(=O)C1CCN(c2nc(-c3ccc(Br)cc3)cs2)CC1] Structure 18 | NCGC00183533-01 | mouse | 1 | 55% | 43% | 49% | 68% | 51% | 59% |
| 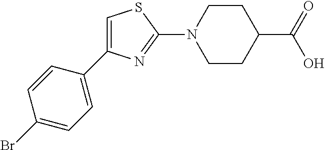 [SMILES: O=C(O)C1CCN(c2nc(-c3ccc(Br)cc3)cs2)CC1] Structure 6 | NCGC00183568-01 | mouse | 1 | 76% | 85% | 81% | 90% | 101% | 95% |
| 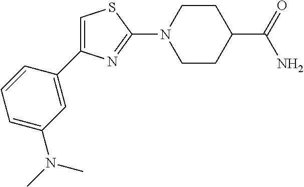 [SMILES: CN(C)c1cccc(-c2csc(N3CCC(C(N)=O)CC3)n2)c1] Structure 7 | NCGC00183564-01 | mouse | 1 | 0% | 0% | 0% | 69% | 66% | 67% |
| 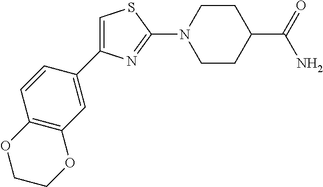 [SMILES: NC(=O)C1CCN(c2nc(-c3ccc4c(c3)OCCO4)cs2)CC1] Structure 17 | NCGC00183579-01 | mouse | 1 | 1% | 0% | 1% | 16% | 15% | 16% |

### Example 168

### Secondary Validation Assays

As initial studies to have a better understanding of the mode of action, we decided to use RT-PCR to measure RNA expression levels and exon 7 inclusion within our reporter cell line (not endogenous SMN). Some analogues showed a slight increase in total SMN-luciferase transcripts but there was almost no increase in exon 7 mRNA (Data not shown). Although the mechanism of SMN protein induction by this class of arylpiperidine analogues was still unclear, this result indicated that SMN activity for this series might be post-transcriptional, potentially stabilizing SMN protein and somehow reducing its degradation.

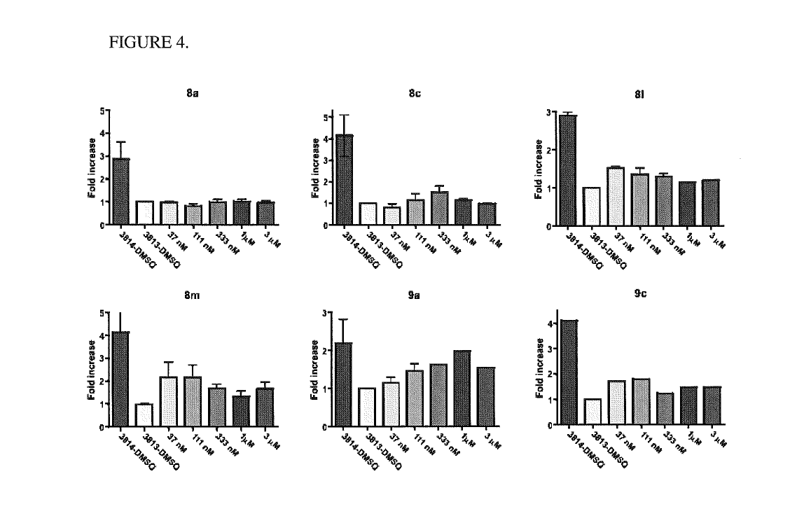

A number of selected analogues featuring desirable potency below 150 nM in the reporter assay were examined to evaluate the effect on the human SMN protein expression using fibroblasts from SMA patients. We incubated a fibroblast cell line from a type I SMA patient (3813 cell line) with different doses of analogues and assessed the SMN protein level by quantitative western blotting (FIG. 4). We observed that analogue 8 m at concentration of 37 nanomolar increased the SMN protein level by 2-3 fold. The SMN protein level was decreased with the increasing of the drug concentration. This result matched with of bell-shaped curve observed in the SMN2-luciferase reporter assay. This could be due with the intrinsic mechanism of action or to other causes, such us concentration dependent compound solubility. Analogue 9a showed a dose-dependent trend between 37 nM to 1 μM concentration and a decrease of protein level at 3 μM. Other selected agents (9c, 8c and 8l) all showed an up-regulation of SMN protein at concentrations ranging between 37 nM and 333 nM.

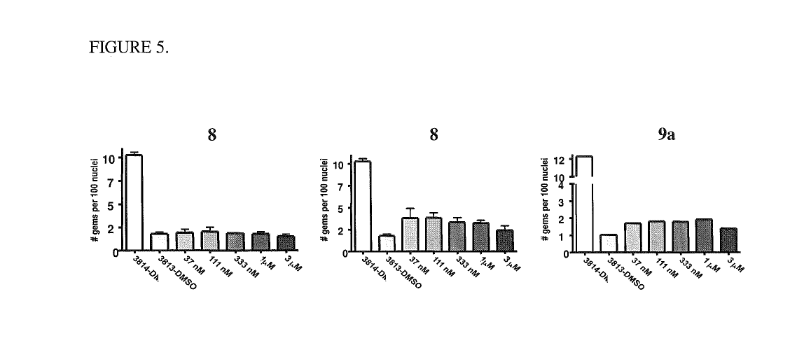

Furthermore, the increase in SMN protein production led us to explore whether the arylpiperidine analogues had effect of on the overall number of SMN positive foci or gems in the nucleus. This would corroborate that the increased protein amounts represent indeed functional SMN protein. SMN protein is predominately a cytoplasmic protein in the nucleous SMN which localized to in distinct punctuate bodies often referred to as gems (gemini of coiled [Cajal] bodies). There is a direct correlation between the number of gems and total SMN protein production in the cell. Gem counts are commonly used as another metric to score the amount total SMN protein expressed on a cell-to-cell basis. The number of nuclei with gems and the number of gems per cell were both significantly reduced in type I SMA cells. Only 3.3% of nuclei have gems in fibroblast cells from SMA type I patients (cell line 3813), while 24.8% of nuclei have gems in fibroblast from a carrier parent (3814 cell line). We treated human type I SMA fibroblasts with increasing doses (37 nM 3000 nM) of arylpiperidine analogues 8a, 8m, and 9a for 3 days and the number of gems per 100 nuclei were examined (FIG. 5). Analogue 8m treatment yielded more than 2 fold of increase of the number of the gems at low 37 nM dose. With the concentration increased, the numbers of gems was reduced which agreed with the previous findings. Analogue 9a also showed an 80% increase of the gem numbers at 37 nM to 1000 nM concentrations and slightly decreased at 3000 nM. However, analogue 8a didn't show any activity in this assay.

What is claimed is:

1. A compound of the formula: 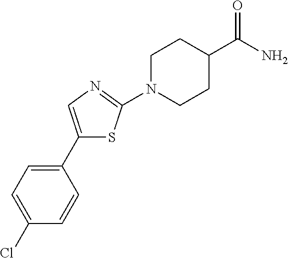 [SMILES: NC(=O)C1CCN(c2ncc(-c3ccc(Cl)cc3)s2)CC1]
or a pharmaceutically acceptable salt thereof, wherein:
X is N or CH;
one of A and B is C—R₁ and the other of A and B is N or C—R₂;
R₁ is an optionally substituted mono-, bi-, or tricyclic group having at least one aromatic or heteroaromatic ring;
R₂ is hydrogen, halogen, C₁-C₂alkyl, C₁C₂alkoxy, trifluoromethyl, or trifluoromethoxy;
R₃ is 1 to 4 substituents independently chosen from hydrogen, halogen, C₁-C₂alkyl, and C₁C₂alkoxy;
R₄ is cyano, amino, (C═O)R₁₀, —(C═O)NR₁₀R₁₁, (C═O)OR₁₀, —NR₁₂(C═O)R₁₀, —NR₁₂(C═O)OR₁₀, or a 5-membered heteroaryl group containing at least 2 nitrogen atoms; wherein
R₁₀ and R₁₁ are independently chosen from hydrogen, C₁-C₄alkyl, (cycloalkyl)C₀-C₄alkyl, (heterocycloalkyl)C₀-C₄alkyl, (phenyl)C₀-C₄alkyl, and (pyridyl)C₀-C₄alkyl; and
R₁₂ is hydrogen or C₁-C₂alkyl, wherein when
R₄ is (C═O)NH₂, R₁ is not unsubstituted phenyl, naphthyl, or phenyl substituted only with halogen, methoxy, or nitro.

2. A compound of the formula: 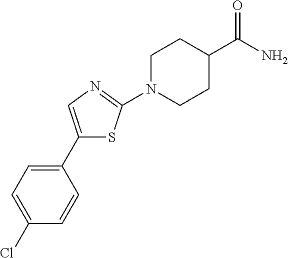 [SMILES: NC(=O)C1CCN(c2ncc(-c3ccc(Cl)cc3)s2)CC1]
or a pharmaceutically acceptable salt thereof, wherein:
X is N or CH;
X is CH, A is CR₂, B is CR₁, D is N, and E is N;
X is CH, A is N, B is CR₁, D is N, and E is N;
X is CH, A is N, B is CR₁, D is N, and E is CR₆;
X is CH, A is N, B is CR₁, D is CR₅, and E is N; or
X is CH, A is R₁, B is CR₂, D is N, and E is N;
R₇ is hydrogen, halogen, C₁-C₂alkyl, or C₁-C₂alkoxy;
R₁ is an optionally substituted mono-, bi-, or tricyclic group having at least one aromatic or heteroaromatic ring;
R₂ is hydrogen, halogen, C₁-C₂alkyl, C₁C₂alkoxy, trifluoromethyl, or trifluoromethoxy;
R₃ is 1 to 4 substituents independently chosen from hydrogen, halogen, C₁-C₂alkyl, and C₁C₂alkoxy;
R₄ is cyano, amino, (C═O)R₁₀, —(C═O)NR₁₀R₁₁, (C═O)OR₁₀, —NR₁₂(C═O)R₁₀, —NR₁₂(C═O)OR₁₀, or a 5-membered heteroaryl group containing at least 2 nitrogen atoms; wherein
R₁₀ and R₁₁ are independently chosen from hydrogen, C₁-C₄alkyl, (cycloalkyl)C₀-C₄alkyl, (heterocycloalkyl)C₀-C₄alkyl, (phenyl)C₀-C₂alkyl, (pyridyl)C₀-C₂alkyl, and thienyl; and
R₁₂ is hydrogen or C₁-C₂alkyl.

3. A compound or salt of claim 1, wherein R₁ is
(i) a 5-membered heteroaryl group containing 1, 2 or 3 heteroatoms chosen from N, O, and S and not more than one O or S heteroatoms:
(ii) phenyl, naphthyl, or a 6-membered heteroaryl group containing one or two nitrogen atoms;
(iii) a phenyl group fused to a 5, 6, or 7-membered heterocycloalkyl group containing one or two heteroatoms independently chosen from N, O, and S;
(iv) a benzofuranyl, indolyl, 9H-fluorenyl, or dibenzo[b,d]thiophenyl group;
each of which (i) and (ii) is substituted with 0 to 3 substituents independently chosen from:
(a) halogen, hydroxy, amino, cyano, nitro, oxo, —(C═O)NH₂, C₁-C₆alkyl, C₁-C₆alkoxy, C₂-C₄alkanoyl, C₁-C₄alkylthio, C₁-C₄alkylsulfonyl, mono- and di-(C₁-C₄alkylamino)C₀-C₂alkyl, mono- and di-(C₁-C₄alkyl)carboxamide, mono- and di-(C₁-C₄alkyl)sulfonamide, C₁-C₂haloalkyl, and C₁-C₂haloalkoxy, and
(b) (C₃-C₆cycloalkyl)C₀-C₂alkyl, (heterocycloalkyl)C₀-C₂alkyl; (phenyl)C₀-C₂alkyl, (phenyl)C₀-C₂alkoxy, and thienyl, each of which (b) is substituted with 0 to 2 substituents independently chosen from halogen, C₁-C₂alkyl, and C₁-C₂alkoxy; and
each of which (iii) and (iv) is substituted with 0 to 4 substituents independently chosen from halogen, C₁-C₂alkyl, C₁-C₂alkyl, mono- and di-(C₁-C₂alkyl)amino, trifluoromethyl, and trifluoromethoxy.

4. A compound or salt of claim 1, of the formula

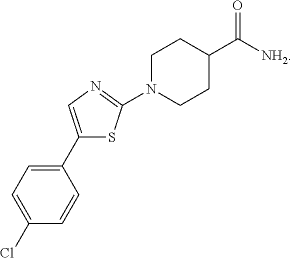 [SMILES: NC(=O)C1CCN(c2ncc(-c3ccc(Cl)cc3)s2)CC1]

5. A compound or salt of claim 1 of the formula

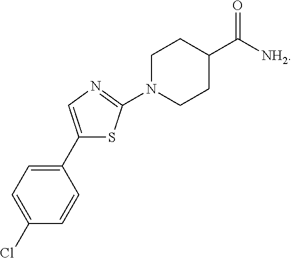 [SMILES: NC(=O)C1CCN(c2ncc(-c3ccc(Cl)cc3)s2)CC1]

6. A compound or salt of claim 1 of the formula

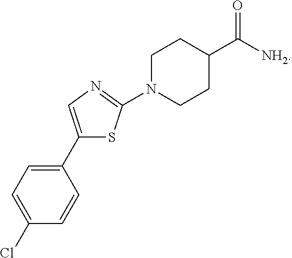 [SMILES: NC(=O)C1CCN(c2ncc(-c3ccc(Cl)cc3)s2)CC1]

7. A compound or salt of claim 2 of the formula

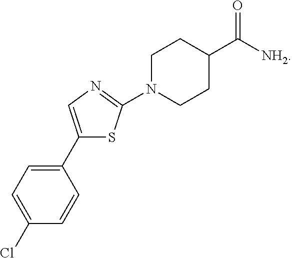 [SMILES: NC(=O)C1CCN(c2ncc(-c3ccc(Cl)cc3)s2)CC1]

8. A compound or salt of claim 2 of the formula

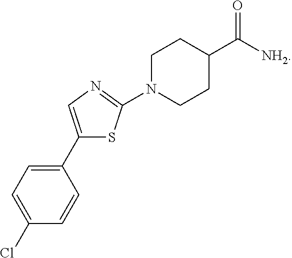 [SMILES: NC(=O)C1CCN(c2ncc(-c3ccc(Cl)cc3)s2)CC1]

9. A compound or salt of claim 2 of the formula

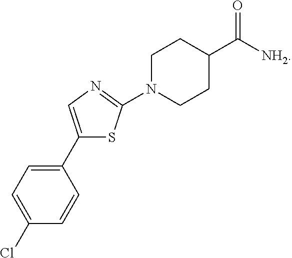 [SMILES: NC(=O)C1CCN(c2ncc(-c3ccc(Cl)cc3)s2)CC1]

10. A compound or salt of claim 2, of the formula

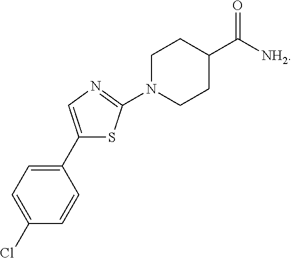 [SMILES: NC(=O)C1CCN(c2ncc(-c3ccc(Cl)cc3)s2)CC1]

11. A compound or salt of claim 2, of the formula

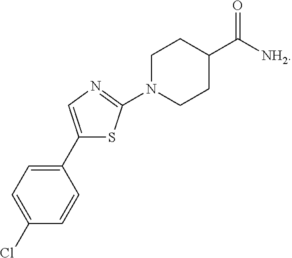 [SMILES: NC(=O)C1CCN(c2ncc(-c3ccc(Cl)cc3)s2)CC1]

12. A compound or salt of claim 1, wherein R₂ is hydrogen and R₃ is hydrogen at each occurrence.

13. A compound or salt of claim 1, wherein R₂ is fluoro and R₃ is hydrogen at each occurrence.

14. A compound or salt of claim 1, wherein R₄ is
R₄ is cyano, amino, (C═O)R₁₀, —(C═O)NR₁₀R₁₁, NR₁₂(C═O)R₁₀, imidazolyl, tetrazolyl; wherein
R₁₀ and R₁₁ are independently chosen from hydrogen, C₁-C₄alkyl, morpholinyl, piperazinyl, and (phenyl)C₀-C₄alkyl; and
R₁₂ is hydrogen or methyl.

15. (canceled)

16. A compound or salt of claim 15, wherein R₄ is —(C═O)OH or —(C═O)NH₂.

17. A compound or salt of claim 1, wherein
R₁ is
(i) a 5-membered heteroaryl group containing 1, 2 or 3 heteroatoms chosen from N, O, and S and not more than one O or S heteroatoms:
(ii) phenyl, naphthyl, or a pyridyl group containing one or two nitrogen atoms;
(iii) a phenyl group fused to a 5, 6, or 7-membered heterocycloalkyl group containing one or two heteroatoms independently chosen from N, O, and S chosen from 2H-benzo[b][1,4]dioxepinyl, 2,3-dihydrobenzo[b][1,4]dioxinyl, benzo[d][1,3]dioxolyl, 2,3-dihydrobenzofuranyl, 4H-chromenyl;
(iv) a benzofuranyl, indolyl, 9H-fluorenyl, or dibenzo[b,d]thiophenyl group;
each of which (i) and (ii) is substituted with 0 to 3 substituents independently chosen from:
(a) halogen, hydroxy, amino, cyano, nitro, oxo, —(C═O)NH₂, C₁-C₄alkyl, C₁-C₄alkoxy, C₂-C₄alkanoyl, C₁-C₂alkylthio, C₁-C₂alkylsulfonyl, mono- and di-(C₁-C₄alkylamino)C₀-C₂alkyl, mono- and di-(C₁-C₂alkyl)carboxamide, trifluoromethyl, trifluoromethoxy, CF₃S— and
(b) piperidinyl, morpholinyl, piperazinyl, pyrrolidinyl, (phenyl)C₀-C₂alkyl, (phenyl)C₀-C₂alkoxy, and thienyl, each of which (b) is substituted with 0 to 2 substituents independently chosen from halogen, C₁-C₂alkyl, and C₁-C₂alkoxy; and
each of which (iii) and (iv) is substituted with 0 to 2 substituents independently chosen from halogen, methyl, and methoxy.

18. A compound or salt of claim 17, wherein
R₁ is phenyl or pyridyl, each of which is substituted with 1 to 3 substituents independently chosen from
(a) halogen, hydroxy, amino, cyano, nitro, —(C═O)NH₂, C₁-C₄alkyl, C₁-C₄alkoxy, C₂-C₄alkanoyl, C₁-C₂alkylthio, C₁-C₂alkylsulfonyl, mono- and di-(C₁-C₄alkylamino), mono- and di-(C₁-C₂alkyl)carboxamide, trifluoromethyl, trifluoromethoxy, CF₃S— and
(b) piperidinyl, morpholinyl, piperazinyl, pyrrolidinyl, (phenyl)C₀-C₂alkyl, (phenyl)C₀-C₂alkoxy, and thienyl, each of which is substituted with 0 to 2 substitutents independently chosen from halogen, C₁C₂alkyl, and C₁-C₂alkoxy.

19. (canceled)

20. A compound or salt of claim 17, wherein
R₁ is a group of the formula 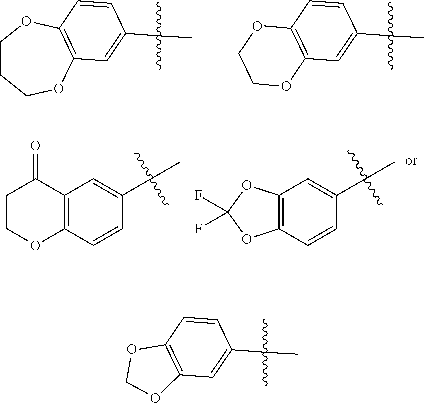 [SMILES: *c1ccc2c(c1)OCCCO2 ; *c1ccc2c(c1)OCCO2 ; *c1ccc2c(c1)C(=O)CCO2 ; *c1ccc2c(c1)OC(F)(F)O2 ; *c1ccc2c(c1)OCO2]
each of which is substituted with 0 to 2 substituents independently chosen from halogen, methyl, and methoxy.

21. A compound or salt of claim 17, wherein R₁ is
a benzofuranyl, indolyl, 9H-fluorenyl, or dibenzo[b,d]thiophenyl group;
each of which is substituted with 0 to 2 substituents independently chosen from halogen, methyl, and methoxy.

22. A compound or salt thereof, wherein the compound is:
1-(4-(4-cyanophenyl)thiazol-2-yl)piperidine-4-carboxamide;
1-(4-(4-fluorophenyl)thiazol-2-yl)piperidine-4-carboxamide;
1-(4-(4-chlorophenyl)thiazol-2-yl)piperidine-4-carboxamide;
1-(4-(4-methylphenyl)thiazol-2-yl)piperidine-4-carboxamide;
1-(4-(4-trifluoromethylphenyl)thiazol-2-yl)piperidine-4-carboxamide;
1-(4-(4-bromophenyl)thiazol-2-yl)piperidine-4-carboxamide;
1-(4-(4-(methylsulfonyl)phenyl)thiazol-2-yl)piperidine-4-carboxamide;
1-(4-(4-methoxyphenyl)thiazol-2-yl)piperidine-4-carboxamide;
1-(4-(3-bromophenyl)thiazol-2-yl)piperidine-4-carboxamide;
1-(4-(2-bromophenyl)thiazol-2-yl)piperidine-4-carboxamide;
1-(4-(biphenyl-4-yl)thiazol-2-yl)piperidine-4-carboxamide;
1-(4-(4-nitrophenyl)thiazol-2-yl)piperidine-4-carboxamide;
1-(4-phenylthiazol-2-yl)piperidine-4-carboxamide;
4-(4-(4-bromophenyl)thiazol-2-yl)morpholine;
4-(4-bromophenyl)-2-(piperidin-1-yl)thiazole;
1-(4-(3-fluorophenyl)thiazol-2-yl)piperidine-4-carboxamide;
1-(4-(3-chlorophenyl)thiazol-2-yl)piperidine-4-carboxamide; 1-(4-(3-methoxyphenyl)thiazol-2-yl)piperidine-4-carboxamide;
1-(4-(3-(methylsulfonyl)phenyl)thiazol-2-yl)piperidine-4-carboxamide;
1-(4-(3-nitrophenyl)thiazol-2-yl)piperidine-4-carboxamide;
1-(4-(2-fluorophenyl)thiazol-2-yl)piperidine-4-carboxamide;
1-(4-(2-chlorophenyl)thiazol-2-yl)piperidine-4-carboxamide;
1-(4-(2-(trifluoromethyl)phenyl)thiazol-2-yl)piperidine-4-carboxamide;
1-(4-(2-methoxyphenyl)thiazol-2-yl)piperidine-4-carboxamide;
1-(4-(2-(methylsulfonyl)phenyl)thiazol-2-yl)piperidine-4-carboxamide;
1-(4-(2-nitrophenyl)thiazol-2-yl)piperidine-4-carboxamide;
ethyl 1-(4-(4-bromophenyl)thiazol-2-yl)piperidine-4-carboxylate;
1-(4-(4-acetylphenyl)thiazol-2-yl)piperidine-4-carboxamide;
1-(4-(benzo[d][1,3]dioxol-5-yl)thiazol-2-yl)piperidine-4-carboxamide;
1-(4-(2,3-dihydrobenzo[b][1,4]dioxin-6-yl)thiazol-2-yl)piperidine-4-carboxamide;
1-(4-(naphthalen-1-yl)thiazol-2-yl)piperidine-4-carboxamide;
1-(4-(4-oxo-4H-chromen-6-yl)thiazol-2-yl)piperidine-4-carboxamide;
1-(4-(thiophen-2-yl)thiazol-2-yl)piperidine-4-carboxamide;
1-(4-(3,5-dimethylisoxazol-4-yl)thiazol-2-yl)piperidine-4-carboxamide;
1-(4-(2-aminophenyl)thiazol-2-yl)piperidine-4-carboxamide;
1-(4-(pyridin-3-yl)thiazol-2-yl)piperidine-4-carboxamide;
1-(4-(3-acetylphenyl)thiazol-2-yl)piperidine-4-carboxamide;
1-(4-(naphthalen-2-yl)thiazol-2-yl)piperidine-4-carboxamide;
1-(4-(3-aminophenyl)thiazol-2-yl)piperidine-4-carboxamide;
1-(4-(3-cyanophenyl)thiazol-2-yl)piperidine-4-carboxamide;
1-(4-(2-(dimethylamino)phenyl)thiazol-2-yl)piperidine-4-carboxamide;
1-(4-(6-(piperidin-1-yl)pyridin-3-yl)thiazol-2-yl)piperidine-4-carboxamide;
1-(4-(4-acetamidophenyl)thiazol-2-yl)piperidine-4-carboxamide;
1-(4-(3-(dimethylcarbamoyl)phenyl)thiazol-2-yl)piperidine-4-carboxamide;
1-(4-(3-(dimethylamino)phenyl)thiazol-2-yl)piperidine-4-carboxamide;
1-(4-(dibenzo[b,d]thiophen-1-yl)thiazol-2-yl)piperidine-4-carboxamide;
1-(4-(1-methyl-1H-indol-5-yl)thiazol-2-yl)piperidine-4-carboxamide;
1-(4-(biphenyl-2-yl)thiazol-2-yl)piperidine-4-carboxamide;
1-(4-(2-cyanophenyl)thiazol-2-yl)piperidine-4-carboxamide;
1-(4-(benzofuran-2-yl)thiazol-2-yl)piperidine-4-carboxamide;
1-(4-(4-bromophenyl)thiazol-2-yl)piperidine-4-carboxylic acid;
1-(4-(4-bromophenyl)thiazol-2-yl)-N-methylpiperidine-4-carboxamide;
1-(4-(4-bromophenyl)thiazol-2-yl)-N,N-dimethylpiperidine-4-carboxamide;
1-(4-(4-bromophenyl)thiazol-2-yl)-N-phenylpiperidine-4-carboxamide;
N-benzyl-1-(4-(4-bromophenyl)thiazol-2-yl)piperidine-4-carboxamide;
(1-(4-(4-bromophenyl)thiazol-2-yl)piperidin-4-yl)(morpholino)methanone;
(1-(4-(4-bromophenyl)thiazol-2-yl)piperidin-4-yl)(piperidin-1-yl)methanone;
(1-(4-(4-bromophenyl)thiazol-2-yl)piperidin-4-yl)(piperazin-1-yl)methanone;
1-(4-(4-bromophenyl)thiazol-2-yl)-N-phenethylpiperidine-4-carboxamide;
1-(4-(4-bromophenyl)thiazol-2-yl)-N-(3-phenylpropyl)piperidine-4-carboxamide;
1-(4-(4-bromophenyl)thiazol-2-yl)-N-(4-phenylbutyl)piperidine-4-carboxamide
1-(4-(4-bromophenyl)pyrimidin-2-yl)piperidine-4-carboxamide;
1-(4-(3-(trifluoromethyl)phenyl)thiazol-2-yl)piperidine-4-carboxamide;
1-(4-(4-aminophenyl)thiazol-2-yl)piperidine-4-carboxamide;
1-(4-(3,4-dimethoxyphenyl)thiazol-2-yl)piperidine-4-carboxamide;
1-(4-(3,4,5-trimethoxyphenyl)thiazol-2-yl)piperidine-4-carboxamide;
1-(4-(3,5-dimethoxyphenyl)thiazol-2-yl)piperidine-4-carboxamide;
1-(4-(4-(methylthio)phenyl)thiazol-2-yl)piperidine-4-carboxamide;
1-(5-(4-bromophenyl)thiazol-2-yl)piperidine-4-carboxamide;
1-(5-(3-(dimethylamino)phenyl)thiazol-2-yl)piperidine-4-carboxamide;
1-(5-(3-(dimethylamino)phenyl)pyrimidin-2-yl)piperidine-4-carboxamide;
1-(4-(3-(dimethylamino)phenyl)pyrimidin-2-yl)piperidine-4-carboxamide;
1-(4-(4-isopropoxyphenyl)thiazol-2-yl)piperidine-4-carboxamide;
1-(4-(4-phenoxyphenyl)thiazol-2-yl)piperidine-4-carboxamide;
1-(4-(2,3-dihydrobenzofuran-6-yl)thiazol-2-yl)piperidine-4-carboxamide;
1-(4-(4-(benzyloxy)phenyl)thiazol-2-yl)piperidine-4-carboxamide;
1-(4-(4-(trifluoromethoxy)phenyl)thiazol-2-yl)piperidine-4-carboxamide;
1-(4-(4-ethoxyphenyl)thiazol-2-yl)piperidine-4-carboxamide;
1-(4-(4-(4-methylpiperazin-1-yl)phenyl)thiazol-2-yl)piperidine-4-carboxamide;
1-(4-(4-tert-butylphenyl)thiazol-2-yl)piperidine-4-carboxamide;
1-(4-(3,4-dihydro-2H-benzo[b][1,4]dioxepin-7-yl)thiazol-2-yl)piperidine-4-carboxamide;
1-(4-(4-(dimethylamino)phenyl)thiazol-2-yl)piperidine-4-carboxamide;
4-(4-bromophenyl)-2-(methylsulfonyl)thiazole;
1-(4-(4-bromophenyl)thiazol-2-yl)piperidine-4-carbonitrile;
tert-butyl 1-(4-(4-bromophenyl)thiazol-2-yl)piperidin-4-ylcarbamate;
1-(2-(3-(dimethylamino)phenyl)pyrimidin-4-yl)piperidine-4-carboxamide;
1-(4-(4-morpholinophenyl)thiazol-2-yl)piperidine-4-carboxamide;
1-(4-(4-isobutoxyphenyl)thiazol-2-yl)piperidine-4-carboxamide;
1-(4-(3-isopropoxyphenyl)thiazol-2-yl)piperidine-4-carboxamide;
4-(4-bromophenyl)-2-(piperazin-1-yl)thiazole;
1-(4-(4-carbamoylphenyl)thiazol-2-yl)piperidine-4-carboxamide;
1-(4-(4-(4-bromophenyl)thiazol-2-yl)piperazin-1-yl)ethanone;
1-(4-(4-bromophenyl)thiazol-2-yl)piperidin-4-amine;
2-(4-(1H-tetrazol-5-yl)piperidin-1-yl)-4-(4-bromophenyl)thiazole;
N-(1-(4-(4-bromophenyl)thiazol-2-yl)piperidin-4-yl)acetamide;
(4-(4-(4-bromophenyl)thiazol-2-yl)piperazin-1-yl)(furan-2-yl)methanone;
(4-(4-(4-bromophenyl)thiazol-2-yl)piperazin-1-yl)(phenyl)methanone;
4-(4-bromophenyl)-2-(4-(phenylsulfonyl)piperazin-1-yl)thiazole;
4-(4-bromophenyl)-2-(4-(methylsulfonyl)piperazin-1-yl)thiazole;
4-(4-(4-bromophenyl)thiazol-2-yl)piperazine-1-carboxamide;
1-(5-p-tolylthiazol-2-yl)piperidine-4-carboxamide;
1-(5-(2,3-dihydrobenzo[b][1,4]dioxin-6-yl)thiazol-2-yl)piperidine-4-carboxamide;
1-(5-(3-fluorophenyl)thiazol-2-yl)piperidine-4-carboxamide;
1-(5-(4-chlorophenyl)thiazol-2-yl)piperidine-4-carboxamide;
1-(5-(3-isopropoxyphenyl)thiazol-2-yl)piperidine-4-carboxamide;
1-(5-(3,4-dihydro-2H-benzo[b][1,4]dioxepin-7-yl)thiazol-2-yl)piperidine-4-carboxamide;
1-(5-(benzo[d][1,3]dioxol-5-yl)thiazol-2-yl)piperidine-4-carboxamide;
1-(4-p-tolylpyrimidin-2-yl)piperidine-4-carboxamide;
1-(4-(3-isopropoxyphenyl)pyrimidin-2-yl)piperidine-4-carboxamide;
1-(4-(3,4-dihydro-2H-benzo[b][1,4]dioxepin-7-yl)pyrimidin-2-yl)piperidine-4-carboxamide;
1-(4-(2,3-dihydrobenzo[b][1,4]dioxin-6-yl)pyrimidin-2-yl)piperidine-4-carboxamide;
1-(5-(3,4-dihydro-2H-benzo[b][1,4]dioxepin-7-yl)-1,3,4-thiadiazol-2-yl)piperidine-4-carboxamide;
1-(5-(4-bromophenyl)-1,3,4-thiadiazol-2-yl)piperidine-4-carboxamide;
1-(5-(3-(dimethylamino)phenyl)-1,3,4-thiadiazol-2-yl)piperidine-4-carboxamide;
1-(5-p-tolyl-1,3,4-thiadiazol-2-yl)piperidine-4-carboxamide;
1-(5-(4-(methylthio)phenyl)thiazol-2-yl)piperidine-4-carboxamide;
2-(4-(1H-imidazol-2-yl)piperidin-1-yl)-5-(3,4-dihydro-2H-benzo[b][1,4]dioxepin-7-yl)thiazole;
3-(2-(4-(1H-imidazol-2-yl)piperidin-1-yl)thiazol-5-yl)-N,N-dimethylaniline;
1-(5-(3-(pyrrolidin-1-yl)phenyl)thiazol-2-yl)piperidine-4-carboxamide;
1-(5-(3-(piperidin-1-yl)phenyl)thiazol-2-yl)piperidine-4-carboxamide;
1-(5-(3-morpholinophenyl)thiazol-2-yl)piperidine-4-carboxamide;
1-(1-(5-(3,4-dihydro-2H-benzo[b][1,4]dioxepin-7-yl)thiazol-2-yl)piperidin-4-yl)ethanone;
1-(1-(5-(3-(dimethylamino)phenyl)thiazol-2-yl)piperidin-4-yl)ethanone;
1-(6-(3,4-dihydro-2H-benzo[b][1,4]dioxepin-7-yl)pyrimidin-4-yl)piperidine-4-carboxamide;
1-(6-(3-(dimethylamino)phenyl)pyrimidin-4-yl)piperidine-4-carboxamide;
1-(4-(3,4-dihydro-2H-benzo[b][1,4]dioxepin-7-yl)-1,3,5-triazin-2-yl)piperidine-4-carboxamide;
1-(4-(3-(dimethylamino)phenyl)-1,3,5-triazin-2-yl)piperidine-4-carboxamide;
ethyl 1-(5-(3,4-dihydro-2H-benzo[b][1,4]dioxepin-7-yl)thiazol-2-yl)piperidine-4-carboxylate;
1-(4-(3-(dimethylamino)phenyl)-5-methylpyrimidin-2-yl)piperidine-4-carboxamide;
1-(5-(3,4-dihydro-2H-benzo[b][1,4]dioxepin-7-yl)thiazol-2-yl)piperidine-4-carboxylic acid;
1-(4-(3-(dimethylamino)phenyl)-5-fluoropyrimidin-2-yl)piperidine-4-carboxamide;
1-(5-(3,4-dimethoxyphenyl)thiazol-2-yl)piperidine-4-carboxamide;
1-(5-(3,4-dihydro-2H-benzo[b][1,4]dioxepin-7-yl)thiazol-2-yl)-N-phenylpiperidine-4-carboxamide;
1-(2-(3-(dimethylamino)phenyl)-5-fluoropyrimidin-4-yl)piperidine-4-carboxamide; or
1-(5-(2,2-difluorobenzo[d][1,3]dioxol-5-yl)thiazol-2-yl)piperidine-4-carboxamide .

23. (canceled)

24. A pharmaceutical composition comprising a compound or salt of claim 1 together with a pharmaceutically acceptable carrier.

25-26. (canceled)

27. A method of increasing SMN2 expression in a mammal comprising administering a compound or salt of claim 1 to the mammal.
